(19) United States
(12) Patent Application Publication
(10) Pub. No.: US 2022/0175734 A1
(43) Pub. Date: Jun. 9, 2022

(54) USING PARASYMPATHOMIMETIC DRUGS ALONE OR, IN COMBINATION WITH ONE OR MORE ALPHA AGONISTS IN PSEUDOPHAKIC PATIENTS, TO CREATE MULTI-FOCALITY

(71) Applicant: Visus Therapeutics, Inc., Seattle, WA (US)

(72) Inventors: Robert P. SAMBURSKY, Lakewood Ranch, FL (US); Herbert E. KAUFMAN, Sarasota, FL (US)

(21) Appl. No.: 17/618,152

(22) PCT Filed: Jun. 10, 2020
(86) PCT No.: PCT/US2020/037042

§ 371 Date: Dec. 10, 2021

Related U.S. Application Data
(60) Provisional application No. 62/859,678, filed on Jun. 10, 2019.
(60) Provisional application No. 62/913,794, filed on Oct. 11, 2019.
(60) Provisional application No. 62/963,891, filed on Jan. 21, 2020.
(60) Provisional application No. 62/970,155, filed on Feb. 4, 2020.

(51) Int. Cl. A61K 31/4178; A61K 31/27; A61K 31/417; A61K 31/498; A61K 47/26; A61K 9/00; A61K 9/08; A61P 27/10

(52) U.S. Cl. CPC A61K 31/4178; A61K 31/27; A61K 31/417; A61P 27/10; A61K 47/26; A61K 9/0048; A61K 9/08; A61K 31/498

(57) ABSTRACT

Using one or more parasympathomimetic drugs alone or together, or in combination with one or more alpha agonists to create optically beneficial miosis to temporarily create multifocality in a pseudophakic patient to treat presbyopia. A pharmaceutical preparation comprising a therapeutically effective amount of one or more parasympathomimetic drugs or cholinesterase inhibitors, alone or in combination with or a pharmaceutically acceptable salt thereof, in combination with one or more alpha agonists or antagonists, or a pharmaceutically acceptable salt thereof. A method for creating multifocality in a pseudophakic patient, reducing symptoms of presbyopia in a patient having an eye or both eyes through administering to an eye or eyes a pharmaceutically effective amount of the ophthalmic preparation is also disclosed.

### BACKGROUND OF THE INVENTION

### Field of the Invention

The invention pertains to the field of treating optical disorders. More particularly, the invention pertains to the use of one or more parasympathomimetic drugs or a cholinesterase inhibitor alone or in combination with one or more alpha agonists or antagonist to create optically beneficial miosis to induce multifocality in a pseudopkaic patient, for example, to temporarily treat presbyopia.

### Description of Related Art

Presbyopia is typically age-related eye deterioration. Young, properly functioning, eyes are able to see at near distances, an ability that deteriorates as one ages.

Presbyopia normally develops as a person ages, and is associated with a natural progressive loss of accommodation with naturally occurring stiffening of the crystalline lens with age. A presbyopic eye loses the ability to rapidly and easily focus on objects at near distances. Presbyopia progresses over the lifetime of an individual, usually becoming noticeable after the age of 45 years. By the age of 65 years, the crystalline lens has often lost almost all elastic properties and has only limited ability to change shape.

Use of over the counter reading glasses is a very common way of addressing the vision problems associated with presbyopia. Reading glasses allow the eye to focus on near objects and maintain a clear image. This approach is similar to that of treating hyperopia, or farsightedness.

Many presbyopes are also prescribed bi-focal eyeglasses, where one portion of the lens is corrected for distance vision and another portion of the lens is corrected for near vision. When peering down through the bifocals, the individual looks through the portion of the lens corrected for near vision. When viewing distant objects, the individual looks higher, through the portion of the bi-focals corrected for distance vision. Contact lenses and intra-ocular lenses (IOLs) have also been used to treat presbyopia, for example, by relying on monovision (where one eye is corrected for distance-vision, while the other eye is corrected for near-vision) or bilateral correction with either bi-focal or multi-focal lenses. Laser ablation has also been used to treat presbyopia. All these procedures seek to correct the problem for long term purposes using drastic steps (surgery, laser ablation, etc.) or require wearing corrective lenses.

Numerous research studies have tried to determine the exact reasons for the development of presbyopia and different attempts have been made to decrease the effects of presbyopia with glasses and contact lenses. These have been of limited usefulness. The use of pinhole spectacles has also been unsatisfactory since the pinhole does not move with the eye and the field of vision of the eye is restricted. Pinhole spectacles often also cause dimness when insufficient light reaches the retina.

Surgical approaches to relieve presbyopia include monovision, laser ablation, intraocular lenses, and refractive lens replacement. Refractive corneal inlays increase the corneal power, usually in the non-dominant eye. These inlays require surgery. An intraocular lens (IOL) is an artificial lens. It replaces the eye's natural lens that is removed during cataract surgery. The most common type of lens used with cataract surgery is called a monofocal IOL. It has one focusing distance. It is set to focus for up close, medium range or distance vision. Most people have them set for clear distance vision. Since these lenses do not correct for presbyopia, patients typically wear eyeglasses for reading or close work after surgery. Special IOLs, called multifocal and accommodative IOLs, have different focusing powers within the same lens. These IOLs reduce the postoperative dependence on reading glasses by providing increased near vision along with distance vision.

Another surgical approach to treat presbyopia is the AcuFocus™ implant, which is a corneal implant with a small central artificial pupil. The AcuFocus implant is similar to a washer with a hole in the middle, which is inserted under the flap of the cornea during a surgical procedure. This procedure restores reading vision through increased depth of focus. Operating only on the non-dominant eye seems to avoid dimness problems that are seen when the pupil in both eyes is made small.

Other refractive errors include myopia (nearsightedness), hyperopia (a condition where rays of light reach the retina before they converge in a focused way, resulting in general blurriness) and astigmatism (an imperfection in the curvature of the eye). Man-made refractive errors or visual distortions also often occur after laser surgery or when the natural lens is replaced with an artificial intraocular lens (for example during cataract surgery).

A cataract is a clouding of the lens in the eye that affects vision. Before a cataract develops, the lens is a clear structure that helps to focus light, or an image, on the retina. Most cataracts are related to aging. Most people develop cataracts after age 50 years, and presbyopia has already occurred. Cataracts are very common in older people. A cataract can occur in either or both eyes.

Canadian patent, CA 2747095, entitled “Optical Correction” by Anant Sharma discusses a medicament for topical administration to the eye to improve visual acuity for several hours and provide benefit to users with presbyopia, myopia, hypermetropia, stigmatism and/or impaired night vision. The medicament includes two pharmacologically active agents—a parasympathetic agonist and either a sympathetic antagonist or a sympathetic agonist. The parasympathetic agonist is pilocarpine, the sympathetic antagonist is selected from dapiprazole or thymoxamine and the sympathetic agonist is selected from brimonidine or iopidine. Eye drop formulations were prepared and tested on three individuals.

Each of the three individuals tested received both the first and the second eye drop formulations. The first eye drop formulation was 0.5% by weight dapiprazole and 0.5% by weight pilocarpine. The second eye drop formulation was 0.1% by weight brimonidine and 0.25% by weight pilocarpine.

Visions tests were conducted before and after the drop formulations were administered. In each case, effects were maintained for at least two hours and some for at least four hours.

The first individual was a 63 year old emmetrope not requiring glasses for functional distance. Within twenty minutes of administration, the patient's unaided distance vision in each eye had improved by a line on the Snellen chart, from 6/6 to 6/5, The refraction did not change. The patient's unaided reading vision improved from N12 to N4.5 at a reading distance of one third of a meter. The patient's night vision improved qualitatively as described by the patient.

The second individual was a 50 year old with a −4 Dioptre myope (requiring glasses for functional distance vision). Within half an hour of administration, the patient's unaided distance vision improved from being able to count fingers (but not to read the Snellen chart) to 6/36 on the Chart. Wearing distance-corrected glasses, the patient's reading vision at a distance of one third of a meter improved from N12 to N4.5. The refraction did not change. Quality of night vision improved as the patient noted less haloes and glare, and night vision also improved quantitatively from 6/6 to 6/5 in dim conditions.

The third individual was a 49 year old with +4 Dioptre hypermetrope (longsighted and requiring glasses for useful reading vision). Within half an hour of administration, the patient's unaided distance vision improved on the Snellen chart from 6/60 to 6/24. The patient's unaided reading vision at one third of a meter improved from N18 to N4.5. The refraction did not change. Quality of night vision improved, the patient noting less haloes and glare, and night vision also improved quantitatively from 6/6 to 6/5 in dim conditions.

The only side effects discussed in CA 2747095 were red eye, which were not suffered by any of the individuals tested.

The inventor of CA 2747095 hypothesized that the combination of the parasympathetic agonists and sympathetic agonists and antagonists have little or no effect on the ciliary muscles of the eye which act to alter the shape and hence refraction of the lens. Ciliary muscles as discussed below however, is responsible for brow ache.

CA 2747095's data is problematic as it was tested on only three individuals, some of which wear corrective lens and some which do not. Additionally, no measurements were taken as to the effects of the drops at time increments after they were received. Furthermore, a control was not tested on the individuals as a comparison to rule out placebo effect.

Thus, there remains a need for new ways of ameliorating or reducing refractive errors including, but not limited to, presbyopia, hyperopia, myopia, pseudophakes, and disruptions due to laser surgery for patients that do not wish to undergo surgery (IOLs, laser ablation, etc.) or use corrective glasses. For people who use corrective lenses, there remains a need to temporarily treat these disorders without the use of corrective lenses.

### SUMMARY OF THE INVENTION

A pharmaceutical preparation includes one or more parasympathomimetic drugs alone or in combination with one or more sympatholytics. Sympatholytics inhibit sympathetic activity and include alpha-1 agonists and alpha-2 agonists. In one embodiment, an ophthalmic topical preparation is provided, comprising a therapeutically effective amount of one or more parasympathomimetic drugs, or pharmaceutically acceptable salts thereof, alone or in combination with and one or more alpha agonists or antagonists, or pharmaceutically acceptable salts thereof which creates optically beneficial miosis to induce multifocality in a pseudopkaic patient, for example, to temporarily treat presbyopia.

Methods for ameliorating or reducing optical errors in patients having at least one eye comprise administering to at least one eye a therapeutically effective amount of an ophthalmic preparation comprising one or more parasympathomimetic drugs, or their pharmaceutically acceptable salts, alone or in combination with and one or more alpha agonists or antagonists, or their pharmaceutically acceptable salts.

Methods for ameliorating or reducing refractive errors and creating multi-focality in pseudophakic patients having at least one eye comprise administering to at least one eye a therapeutically effective amount of an ophthalmic preparation comprising one or more parasympathomimetic drugs, or their pharmaceutically acceptable salts, alone or in combination with and one or more alpha agonists or antagonists, or their pharmaceutically acceptable salts.

The invention also provides for a method of producing ocular miosis in a subject which comprises administering to the subject an amount of a preparation comprising one or more parasympathomimetic, or their pharmaceutically acceptable salts, and one or more alpha agonists or antagonists, or their pharmaceutically acceptable salts, effective to produce ocular miosis and one or more alpha agonists or antagonists.

The invention also provides for a method of producing ocular miosis to induce multi-focality in a subject which comprises administering to the subject an amount of a preparation comprising one or more parasympathomimetic, or their pharmaceutically acceptable salts, alone or in combination with and one or more alpha agonists or antagonists, or their pharmaceutically acceptable salts, effective to produce ocular miosis and one or more alpha agonists or antagonists.

A method for ameliorating or reducing at least one refractive error selected from the group consisting of myopia, hyperopia, pseudophakia and astigmatism of a patient includes administering to at least one eye of the patient an ophthalmic preparation comprising a therapeutically effective amount of one or more parasympathomimetic drugs, or pharmaceutically acceptable salts thereof; and a therapeutically effective amount of an alpha agonist or an alpha antagonist, or pharmaceutically acceptable salts thereof.

A method of inducing multi-focality in a subject through ocular miosis of patient with at least one refractive error selected from the group consisting of myopia, hyperopia, and astigmatism of a pseudophakic patient includes administering to at least one eye of the patient an ophthalmic preparation comprising a therapeutically effective amount of one or more parasympathomimetic drugs, or pharmaceutically acceptable salts thereof alone or in combination with a therapeutically effective amount of an alpha agonist or an alpha antagonist, or pharmaceutically acceptable salts thereof.

A method of treating at least one refractive error in a patient that has had ocular surgery includes administering to at least one eye of the patient an ophthalmic preparation comprising a therapeutically effective amount of one or more parasympathomimetic drugs, or pharmaceutically acceptable salts thereof; and a therapeutically effective amount of an alpha agonist or an alpha antagonist, or pharmaceutically acceptable salts thereof. In some embodiments, the refractive error may include, but is not limited to, myopia, hyperopia, astigmatism, and any combination of myopia, hyperopia, and astigmatism in a pseudophakic patient. The ocular surgery can include cataract surgery, surgery to alter at least one eye with an intraocular lens or lens replacement.

A method of treating pseudophakia in a patient includes administering to at least one eye of the patient an ophthalmic preparation comprising a therapeutically effective amount of one or more parasympathomimetic drugs, or pharmaceutically acceptable salts thereof; alone or in combination with a therapeutically effective amount of an alpha agonist or an alpha antagonist, or pharmaceutically acceptable salts thereof.

In some embodiments, the one or more parasympathomimetic drugs is carbachol or pilocarpine, and the alpha agonist is brimonidine or phentolamine.

In some embodiments, the alpha agonist is brimonidine, or its pharmaceutically acceptable salt, is present in an amount less than about 0.05%, 0.2%, 0.15% or 0.10%. In other embodiments, the alpha antagonist is phentolamine, or its pharmaceutically acceptable salt, is present in an amount of less than 2%. In some further embodiments, the one or more parasympathomimetic drugs is carbachol, or its pharmaceutically acceptable salt, which is present in the preparation in an amount of about 0.50%-5%. In other embodiments, the one or more parasympathomimemtic drugs is pilocarpine, or its pharmaceutically acceptable salt, which is present in the preparation in an amount of about 0.25%-1.5%. In yet other embodiments, the one or more parasympathomimemtic drugs is pilocarpine, or its pharmaceutically acceptable salt, which is present in the preparation in an amount of about 0.25%-4.0%. In other embodiments, the pilocarpine, or its pharmaceutically acceptable salt, is present in an amount of less than 0.1%.

In some embodiments, the ophthalmic preparation includes a permeation enhancer such as benzalkonium chloride (BAC or BAK) in an amount of 0.005-0.3%. More preferably, benzalkonium chloride is present in an amount of 0.005-0.1%.

Use of alpha adrenergic simulation such as from brimonidine can be used to improve scotopic and mesopic vision when combined with topical parasympathomimetics medications for pseudophakia.

### BRIEF DESCRIPTION OF THE DRAWING

FIG. 1 shows change in visual acuity at 1 hr, 2 hrs, and 4 hrs after administration of 0.25% pilocarpine alone, 0.5% pilocarpine alone, 1.0% pilocarpine alone, 0.25% pilocarpine combined with 0.2% brimonidine, 0.5%, pilocarpine combined with 0.2% brimonidine, or 1.0% pilocarpine combined with 0.2% brimonidine.

FIG. 2 shows the average change in visual acuity at 1, 2, 4, 8, and 10 hours after administration for the active drug and placebo arms. The solid squares represent the average change in visual acuity for the active drug arm whereas the solid triangles represent the average change in visual acuity for the placebo arm.

FIG. 3 shows the distribution of mean change in near visual acuity (Jaeger) over time for presbyopic subjects ≥50 years (2.25% carbachol plus brimonidine versus placebo).

FIG. 4 shows in near visual acuity (Jaeger) over time for presbyopic subjects <50 years (2.25% carbachol plus brimonidine versus placebo).

FIG. 5 shows the distribution of mean change in near visual acuity (J) over time for emmetropic presbyopes (carbachol 2.25% plus brimonidine vs placebo vs brimonidine).

FIG. 6 shows the distribution of mean change in near visual acuity (J) over time for myopic presbyopes (carbachol 1.5% plus brimonidine vs placebo vs brimonidine)

FIG. 7 shows the distribution of mean change in near visual acuity (J) over time for hyperopic presbyopes (carbachol 3% plus brimonidine vs placebo vs brimonidine)

FIGS. 8a-8b shows the data from a study comparing 3% carbachol plus 0.2% brimonidine eye drops administered to the same subjects in a combined formulation versus separate administration.

FIG. 9 shows the distribution of mean change in near visual acuity (J) over time for the same presbyopic subjects receiving 3% carbachol plus 2% brimonidine in both combined and separate forms.

FIG. 10 shows the distribution of mean change in pupil size (mm) over time for the same presbyopic subjects receiving 3% carbachol plus 2% brimonidine in both combined and separate forms.

FIG. 11 shows the distribution of mean change in near visual acuity (J) over time in all groups.

FIG. 12 shows the relationship between change in pupil diameter and Near LogMar VA.

FIG. 13 shows the change in LogMar near UCVA from pretreatment baseline.

FIG. 14 shows the change from baseline near LogMar VA for carbachol plus brimonidine (0.2%) compared to placebo.

FIG. 15 shows the side effects of carbachol plus brimonidine over seven days.

FIG. 16 shows the responses to a survey regarding whether patients would use the drops in the future.

FIG. 17 shows visual measures of PD (pupil dilation) over time for B (brimonidine), P (pilocarpine) and PB (brimonidine plus pilocarpine).

FIG. 18 shows visual measures of NV (near vision) over time for B (brimonidine), P (pilocarpine) and PB (brimonidine plus pilocarpine).

FIG. 19 shows visual measures of IV (intermediate vision) over time for B (brimonidine), P (pilocarpine) and PB (brimonidine plus pilocarpine).

FIG. 20 shows the responses to a survey regarding whether patients would use the drops in the future.

FIG. 21 shows data from 15 patients treated with combination drops after intraocular lens replacement.

FIG. 22 shows distribution of mean change in near visual acuity (Jaeger) over time for group 1 receiving 2.25% carbachol plus brimonidine versus group 2 receiving 3% carbachol plus brimonidine.

FIG. 23 shows distribution of mean change in pupil size (Jaeger) over time for group 1 receiving 2.25% carbachol plus brimonidine versus group 2 receiving 3% carbachol plus brimonidine.

FIG. 24 shows mean pupil size over time for emmetropic presbyopic subjects receiving combined 3% carbachol plus 0.2% brimonidine with 100 ppm of benzalkonium chloride drops, separate administration of 3% carbachol with 50 ppm of benzalkonium chloride and then administration of 0.2% brimonidine, administration of just 3% carbachol with 50 ppm of benzalkonium chloride, and administration of just 0.2% brimonidine with 50 ppm.

FIG. 25 shows mean near visual acuity in emmetropic presbyopes over time for subjects receiving combined 3% carbachol plus 0.2% brimonidine with 100 ppm of benzalkonium chloride drops, separate administration of 3% carbachol with 50 ppm of benzalkonium chloride and then administration of 0.2% brimonidine, administration of just 3% carbachol with 50 ppm of benzalkonium chloride, and administration of just 0.2% brimonidine with 50 ppm.

FIG. 26a shows effect of 2.25% carbachol with 0.2% brimonidine versus 3% carbachol with 0.2% brimonidine over time on pupil size.

FIG. 26b shows effect of 2.25% carbachol with 0.2% brimonidine versus 3% carbachol with 0.2% brimonidine over time on near visual acuity.

FIG. 27 shows a distribution of mean change in near visual acuity for treatment and control groups.

FIG. 28 shows a distribution of mean change in pupil size over time for treatment and control groups.

### DETAILED DESCRIPTION OF THE INVENTION

U.S. Pat. No. 8,299,079, PREPARATIONS AND METHODS FOR AMELIORATING OR REDUCING PRESBYOPIA, issued Oct. 30, 2012 and U.S. Pat. No. 8,455,494, PREPARATIONS AND METHODS FOR AMELIORATING OR REDUCING PRESBYOPIA, issued Jun. 4, 2013, and US Patent Publication Nos. 2010/0298335, PREPARATIONS AND METHODS FOR AMELIORATING OR REDUCING PRESBYOPIA, published Nov. 25, 2010 and 2013/0245030, PREPARATIONS AND METHODS FOR AMELIORATING OR REDUCING PRESBYOPIA, published Sep. 19, 2013, all herein incorporated by reference, discuss methods and preparations to reduce presbyopia using parasympathomimemtic drugs and alphagonists.

In embodiments described herein, an ophthalmic topical preparation is provided comprising a therapeutically effective amount of one or more parasynmpathominimetic drugs or one or more cholinesterase inhibitors, or their pharmaceutically acceptable salts, and one or more alpha agonists or antagonists, or their pharmaceutically acceptable salts.

In some embodiments, the one or more parasympathomimetic drugs is pilocarpine, or carbachol, or their pharmaceutically acceptable salts. In a further embodiment, the one or more alpha agonists is brimonidine, or a pharmaceutically acceptable salt thereof. In a further embodiment, the one or more parasympathomimetic drugs are replaced with a cholinesterase inhibitor.

In some embodiments, the one or more cholinesterase inhibitor is an organophosphate such as metrifonate, a carbamate such as physostigmine (also known as eserine), neostigmine (also known as prostigmine), pyridostigmine, ambenonium, demecarium, or rivastigmine; a phenanthrene derivative such as galantamine; a piperidine compound such as donepezil, tacrine (also known as tetrahydroaminoacridine (THA′)), edrophonium, huperzine A, or ladostigil. In another embodiment, the cholinesterase inhibitor may be diisopropyl fluorophosphate or DFP (Floropryl). In other embodiments, the one or more cholinesterase inhibitors is phospholine iodide (also known as echothiophate) or physostigmine, or its pharmaceutically acceptable salt.

In certain embodiments, the one or more alpha antagonists is doxazosin, silodosin, prazosin, tamsulosin, Alfuzosin, terazosin, trimazosin, phenoxybenzamine, or phentolamine, thymoxamine or a pharmaceutically acceptable salt thereof.

In embodiments described herein, pharmaceutical preparations comprise one or more parasympathomimetic drugs (also known as muscarinic agonists), or cholinesterase inhibitors, alone or in combination with one or more alpha agonists. In one embodiment, the one or more parasympathomimetic drug is pilocarpine. In another embodiment, one or more parasympathomimetic drug is carbachol. In further embodiments, the one or more parasympathomimetic drugs are pilocarpine and carbachol, or a pharmaceutically acceptable salt thereof. In certain embodiments, the one or more alpha agonists is brimonidine, or phentolamine or a pharmaceutically acceptable salt thereof.

The ophthalmic preparation may be administered to a subject suffering from myopia, hyperopia, astigmatism, presbyopia or other optical errors as often as needed to cause miosis sufficient to temporarily treat, ameliorate, or reduce these optical errors as well as temporarily create multifocality. These refractive errors all benefit from these drugs to a clinically and practically usable degree which enable patients who needed glasses full time to totally do without them. Thus, the invention further provides a method for temporarily treating, ameliorating, or reducing these optical errors by inducing miosis as well as temporarily creating multifocality.

“Optical errors”, or “refractive errors”, as defined herein, also known as ammetropia (vision abnormalities), are vision defects or optical imperfections that prevent the eye from properly focusing light, causing blurred vision. The primary refractive errors are myopia (nearsightedness), hyperopia (farsightedness, blurred vision), presbyopia (when the lens in the eye loses flexibility), pseudophakia (a near vision defect created by the implantation of an artificial intraocular lens) and astigmatism (including regular astigmatism, irregular astigmatism and high degrees of regular astigmatism). Some refractive errors occur after cataract surgery or laser surgery.

As used herein, the term “parasympathomimetic agent or drug” or “muscarinic agonist” is intended to include any cholinergic drug that enhances the effects mediated by acetylcholine in the central nervous system, the peripheral nervous system, or both. Examples of these so-called acetylcholine receptor agonists suitable for the preparations and methods of the present invention include acetylcholine, muscarine, pilocarpine, nicotine, suxamethonium, bethanechol, carbachol, methacholine, phenylpropanolamine, amphetamine, ephedrine, phentolamine, and fenfluramine.

As used herein, the term “alpha agonist” or “alpha blocker” refers to compounds that preferentially stimulate alpha (both alpha1 and alpha2) adrenoceptors. Examples of alpha androgenic agonist suitable for the preparations and methods of the present invention include amiloride, apraclonidine, brimonidine, clonidine (and its derivatives such as p-chloro and amino derivatives), detomidine, dexmedetomidine, dipivalylepinephrine, epinephrine, guanabenz, guanfacine, isoproterenol, medetomidine, metaproterenol, mephentermine, methoxamine, methyldopa, naphazoline, norepinephrine, phentolamine, phenylephrine, rilmenidine, salbutamol, terbutaline, tetrahydrozoline, xylazine, thymoxamine, and their pharmaceutically acceptable salts and prodrugs.

In the subject invention a “therapeutically effective amount” is any amount of the one or more active ingredients present in the preparation of the present invention which, when administered to a subject suffering from a refractive error are effective to cause miosis sufficient to temporarily reduce, ameliorate, or treat the refractive error such that the vision of the treated eye is temporarily restored partially or completely. A complete restoration of vision should be sufficient to allow the person to read a Times New Roman font of size 12 without any other aid at a near distance or a far distance, depending upon the refractive error being treated. A partial restoration of near vision will allow the treated eye to see with decreased blurriness. Furthermore, a “therapeutically effective amount” is any amount of the one or more active ingredients present in the preparation of the present invention which, when administered to a subject suffering from a refractive error are effective to cause miosis sufficient to temporarily reduce, ameliorate, or treat the refractive error such that the multifocality of the treated eye is temporarily restored partially or completely. Multifocality is restored if more than focal length is observed by person and the person has better than Jaeger 5 or 20/50 near vision.

Thus, a therapeutically effective amount refers to the amount of a therapeutic preparation that reduces the extent of the refractive error by at least 10%, at least 20%, at least 30%, at least 40%, at least 50%, at least 60%, at least 70%, at least 80%, at least 90%, or at least 100%. For certain embodiments, the amount of the ophthalmic preparation comprising the one or more parasympathomimetic drugs and the one or more alpha agonists is effective to ameliorate or reduce the refractive error for about 12 hours, 11 hours, 10 hours, 9 hours, 8 hours, 7 hours, 6 hours, 5 hours, 4 hours, 3 hours, 2 hours or 1 hour. The extent of presbyopia can be determined by any method known in the art for ophthalmic examination.

For certain embodiments, the amount of the ophthalmic preparation comprising the one or more parasympathomimetic drugs, alone or in combination with the one or more alpha agonists is effective to restore multifocality is for about 8 hours, 7 hours, 6 hours, 5 hours, 4 hours, 3 hours, 2 hours or 1 hour.

In some of these embodiments, the parasympathomimetic drug is carbachol and the alpha agonist is brimonidine. In some embodiments, the parasympathomimetic drug is carbachol and the alpha antagonist is phentolamine. In some embodiments, the parasympathomimetic drug is pilocarpine and the alpha antagonist is brimonidine. In some embodiments, the parasympathomimetic drug is pilocarpine and the alpha antagonist is phentolamine. In some embodiments, the concentration of carbachol is 0.5-50%. In other embodiments, the concentration of carbachol is 2-3%. In some embodiments, the concentration of pilocarpine is less than 0.1%. In some other embodiments, the pilocarpine is less than 4%. In some embodiments, the concentration of brimonidine is 0.05-0.2%. In some embodiments, the concentration of phentolamine is less than 2%.

In some embodiments treating myopes or hyperopes, the concentration of carbachol is preferably approximately 0.5%-5.0%. In some embodiments treating myopes or hyperopes, the concentration of carbachol is preferably approximately 3.0% or less. In some embodiments treating myopes, the concentration of carbachol is preferably approximately 1.5% or less.

In some embodiments, brimonidine, or a pharmaceutically acceptable salt thereof, is present in an amount less than about 0.2%. In other exemplary embodiments, the one or more parasympathomimetic drugs is pilocarpine, or its pharmaceutically acceptable salt, which is present in the preparation in an amount less than about 0.5%. In further exemplary embodiments, the one or more parasympathomimetic drugs is pilocarpine, or its pharmaceutically acceptable salt, which is present in the preparation in an amount of less than about 0.1%.

In some further embodiments, the one or more parasympathomimetic drugs is carbachol, or its pharmaceutically acceptable salt, which is present in the preparation in an amount of about 5%. In certain embodiments, the one or more parasympathomimetic drugs is carbachol, or its pharmaceutically acceptable salt, which is present in the preparation in an amount of no more than 0.001%.

In some further embodiments, the one or more alpha antagonist is phentolamine, or its pharmaceutically acceptable salt, which is present in the preparation in an amount of no more than 2%. In certain embodiments, the one or more alpha antagonist is phentolamine, or its pharmaceutically acceptable salt, which is present in the preparation in an amount of no more than 0.005%.

In some embodiments, the alpha antagonist is thymoxamine or its pharmaceutically acceptable salt, which is present in the preparation in an amount of no more than 2%.

The terms “ameliorate, ameliorating, and amelioration,” as used herein, are intended to refer to a decrease in the severity of the refractive error. The amelioration may be complete, e.g., the total absence of one or more refractive errors. The amelioration may also be partial, such that the amount of the refractive error is less than that which would have been present without the treatment. For example, the extent of the refractive errors-using the methods of the present invention may be at least 10%, at least 20%, at least 30%, at least 40%, at least 50%, at least 60%, at least 70%, at least 80%, at least 90%, or at least 100% less than the amount of the refractive errors that would have been present without using these methods.

Methods described herein ameliorate refractive errors, including, but not limited to, myopia, hyperopia, astigmatism, presbyopia, pseudophakes (replacing a natural lens with an artificial intraocular lens, for example after cataract surgery), and distortions after laser surgery by administering to at least one eye of a patient a therapeutically effective amount of an ophthalmic preparation comprising one or more parasympathomimetic drugs, or pharmaceutically acceptable salts thereof, and one or more alpha agonists or antagonists, or pharmaceutically acceptable salts thereof. In some preferred embodiments, a single ophthalmic preparation includes a parasympathomimetic drug and an alpha agonist or antagonist. In some of these embodiments, the parasympathomimetic drug is carbachol and the alpha agonist is brimonidine. In some embodiments treating myopes or hyperopes, the concentration of carbachol is preferably approximately 0.5%-5.0%. In some embodiments treating myopes or hyperopes, the concentration of carbachol is preferably approximately 3.0% or less. In some embodiments treating myopes, the concentration of carbachol is preferably approximately 1.5% or less.

A pinhole camera cuts down on the amount of light going in. Since the more light you let in requires more focus, the user hardly needed to focus with the pinhole cameras. Using a pinhole removes the optical periphery. The treatment methods and compositions described herein use drugs to get the pinhole effect, thus increasing depth of focus dramatically. There are two types of muscles in the eyes: constrictor muscles and dilator muscles. By acting on both of these types of muscles, the unique combination of drugs described herein are able to accomplish a pinhole effect, correcting refractive errors.

A pharmacologic pinhole effect is induced in at least the non-dominant eyes of any patients with refractive errors. In some embodiments, the treatment may be administered in both eyes. In some preferred embodiments, the treatment is only administered in the non-dominant eye of emmetropic presbyopes and myopic presbyopes and in both eyes of hyperopic presbyopes and hyperopes. For pure myopes, the pinhole effect may be induced in either the non-dominant eye or both eyes of the myopes.

More specifically, parasympamimetic compounds cause the pupil to become small (constriction), and brimonidine acts as a paralyzer (preventing dilation). Brimonidine prevents pupillary dilation, that occurs at night, to minimize optical aberrations causing halos and glare in some patients after refractive surgery, as well as to treat glaucoma.

For some of the refractive errors including, but not limited to, presbyopia, the formulations are placed in only one eye, to decrease the likelihood of dimness from the treatment. By placing the drops in only one eye, the brain fills in the details that you are getting with the treated eye while the other eye receives the light. For other refractive errors including, but not limited to, hyperopia, the drops are preferably placed in both eyes during treatment, but may alternatively be placed only in a single eye. In patients with myopic vision, pseudophakes, or astigmatism, the formulations may be placed in a single eye or in both eyes.

In one embodiment, methods reduce or eliminate dimness of vision of a patient having an eye comprising administering to said eye a therapeutically effective amount of an ophthalmic preparation comprising one or more parasympathomimetic drugs, or pharmaceutically acceptable salts thereof, and one or more alpha agonists or antagonists, or pharmaceutically acceptable salts thereof.

In some embodiments, the invention is directed to a method of improving focus and/or correcting refractive errors of a patient having an eye comprising administering to said eye a therapeutically effective amount of an ophthalmic preparation comprising one or more parasympathomimetic drugs, or pharmaceutically acceptable salts thereof, and one or more alpha agonists or antagonists, or pharmaceutically acceptable salts thereof.

For some refractive errors, it may also be beneficial to administer the pharmaceutical preparations described herein to only a single eye of a patient. In some instances, blurring of distance vision (a result of accommodative focus) and dimness of vision (a result of pupil constriction) may occur when the compositions are administered to both eyes of a patient. When applied to only a single eye, the benefits of improvement in presbyopia are obtained with diminished or complete relief of blurring and dimness. It was originally believed that a patient's brain compensates between the treated and untreated eyes thereby reducing the undesired effects. Therefore, the combination of a constricted pupil with its increased depth of field in the treated eye and normal distance vision and brightness in the untreated eye will cause the brain to ignore any monocular blur at distance or near vision when only one eye was treated. However, when the pharmaceutical preparation is applied to both eyes, distance visual acuity is preserved, even though parasympathomimetic drugs, or pharmaceutically acceptable salts thereof, such as pilocarpine and carbachol alone cause a myopic shift, inducing near sightedness at the expense of distance vision while providing increased depth of focus. The addition of an alpha2 agonist such as brimonidine or its pharmaceutically acceptable salt prevents the myopic shift and preserves distance visual acuity when applied to both eyes.

Although brimonidine is not ordinarily used to constrict the pupil, and thus enhance visual acuity, applicants discovered that it potentiates the effect of pilocarpine or carbachol on the pupil. Thus, an embodiment of the present application is a method for ameliorating or reducing refractive errors of a patient by applying to the one or both eyes of the patient a therapeutically effective amount of pilocarpine, or pharmaceutically acceptable salts thereof, and an effective amount of brimonidine, or pharmaceutically acceptable salts thereof or phentolamine, or pharmaceutically acceptable salts thereof.

Another embodiment of the present application is a method for ameliorating or reducing one or more refractive errors of a patient by applying to the one or both eyes of the patient a therapeutically effective amount of carbachol or pilocarpine, or pharmaceutically acceptable salts thereof, and an effective amount of brimonidine, or pharmaceutically acceptable salts thereof.

Brimonidine should also potentiate the effect on the pupil of other parasympathomimetic drugs such as acetylcholine, muscarine, nicotine, suxamethonium, bethanechol, methacholine, phenylpropanolamine, amphetamine, ephedrine, phentolamine, and fenfluramine).

In some embodiments, the two drugs are administered as a single combined ophthalmic preparation. In another embodiment, the two drugs are formulated as two separate ophthalmic preparations and applied to the eye successively or simultaneously.

In embodiments with a single combined preparation of carbachol and brimonidine, the concentration of carbachol in the preparation is preferably approximately 0.1% to 5.0% and the concentration of brimonidine in the preparation is preferably approximately 0.20% or less. In some preferred embodiments, the brimonidine concentration is approximately 0.15% or less. In other preferred embodiments, the brimonidine concentration is approximately 0.10% or less. In some preferred embodiments, the carbachol concentration is approximately 3.0% or less. In some embodiments, the carbachol concentration is 5% or less. The combined preparation also preferably includes penetration enhancers. In some embodiments, the penetration enhancers include, but are not limited to, one or more of carboxymethylcellulose, BAK, nanoparticles, bycrobextrians, and EDTA. In some embodiments, the combined preparations also include tropicamide. Combined preparations are more effective at ameliorating the refractive errors than carbachol and brimonidine drops given separately.

In some embodiments, the preparation can also include permeation enhancers and excipients to increase the efficacy and reduce ocular surface toxicity and increase tolerability. In some embodiments, the permeation enhancer is BAC in an amount of 0.1-0.3%.

In embodiments with a single combined preparation of pilocarpine and brimonidine, the concentration of pilocarpine is preferably less than 0.1% and the concentration of brimonidine in the preparation is preferably approximately 0.20% or less. In some preferred embodiments, the brimonidine concentration is approximately 0.15% or less. In other preferred embodiments, the brimonidine concentration is approximately 0.10% or less. In yet other preferred embodiments, the brimonidine is 0.05%. The combined preparation can additionally include other elements, such as penetration enhancers.

In embodiments with a single combined preparation of pilocarpine and phentolamine, the concentration of pilocarpine is preferably less than 0.1%. The combined preparation can additionally include other elements, such as penetration enhancers.

In embodiments with a single combined preparation of carbachol and phentolamine, the concentration of carbachol in the preparation is preferably approximately 0.5% to 5.0% and the concentration of phentolamine in the preparation is preferably approximately 2.0% or less. In some preferred embodiments, the carbachol concentration is approximately 3.0% or less. In some embodiments, the carbachol concentration is 5% or less. The combined preparation can additionally include other elements, such as penetration enhancers.

In some embodiments, the preparations used in treatment include tropicamide to reduce symptoms of brow ache. Brow ache is commonly caused by a ciliary muscle spasm which affect the zonular fibers in the eye. The zonular fibers suspend the lens in position during accommodation and enable changes in the lens shape for light focusing. In some embodiments, the concentration of tropicamide is between approximately 0.01% to about 0.10% w/v. In some preferred embodiments, the concentration of tropicamide is between approximately 0.25% to about 0.080% w/v. In other preferred embodiments, the concentration of tropicamide is between approximately 0.04% to about 0.06% w/v.

The pharmaceutical preparations described herein are adapted for topical administration to the eye in the form of solutions, suspensions, ointments, or creams. Alternatively, the ophthalmic pharmaceutical preparation may be used in the form of an eyewash, ophthalmic solution (e.g., eye drop), or ophthalmic ointment.

Ophthalmic pharmaceutical preparations may be prepared using commonly used pharmaceutically-acceptable carriers in such a manner of mixing them with an effective amount of the one or more parasympathomimetic drugs and one or more alpha agonists to suit the desired formulation. The carriers used for ophthalmic solutions and eyewashes include any one of those which are commonly used therefor, usually, purified water. The ophthalmic pharmaceutical preparation can be previously prepared into a solution form or processed into a solid preparation using lyophilization method, etc., to be used in the desired preparation, for example, dissolving the solid preparation in the desired liquid carrier. Examples of such a solid preparation include tablets, granules, and powders. These ophthalmic pharmaceutical preparations can be prepared in accordance with conventional methods and should preferably be sterilized before use by conventional methods using membrane filters, autoclaves, etc. The ophthalmic preparations may comprise saccharides such as glucose and maltose; sugar alcohols such as mannitol and sorbitol; electrolytes such as sodium chloride, sodium hydrogenphosphate, potassium chloride, magnesium sulfate, and calcium chloride; amino acids such as glycine and alanine; vitamins and derivatives thereof such as thiamine hydrochloride, sodium riboflavin phosphate, pyridoxine hydrochloride, nicotinamide, folic acid, biotin, vitamin A, L-ascorbic acid, and alpha.-glycosyl-L-ascorbic acid, which all can be used in an appropriate combination. Particularly, in the case of the ophthalmic pharmaceutical preparation of the present invention in the form of an ophthalmic solution, the combination use of the one or more parasympathomimetic drugs and one or more alpha agonists as an effective ingredient and one or more other saccharides selected from monosaccharides such as glucose and fructose, disaccharides such as maltose, and oligosaccharides higher than maltotriose may stably exert a satisfactory therapeutic effect. In addition, viscosity-imparting agents such as methyl cellulose, carboxy methylcellulose, chondroitin sulfate, polyvinyl alcohol, and pullulan. Solubilizers such as polysorbate 80 may be used in the preparations.

In some preferred embodiments, penetration enhancers including, but not limited to, carboxymethylcellulose, EDTA, nanoparticles, bycrobextrians, and BAK are included in the ophthalmic pharmaceutical preparations. In some of these embodiments, only one of these enhancers are used. In other embodiments, two of these enhancers are used. In still other embodiments, all three of these enhancers are used. In embodiments with nanoparticles, the drops are incorporated into the nanoparticles to increase penetration. BAC is a quaternary ammonium compound used in pharmaceutical formulations as an antimicrobial preservative in applications similar to other cationic surfactants.

In some embodiments, carbachol at 1-3%, or a pharmaceutically acceptable salt thereof, in combination with brimonidine 0.5-0.2%, or a pharmaceutically acceptable salt thereof, specifically combined with permeation enhancer with higher concentrations of benzalkonium chloride of 0.01-0.3%.

In some embodiments, carbachol, or a pharmaceutically acceptable salt thereof, specifically is combined with permeation enhancer with higher concentrations of benzalkonium chloride of 0.01-0.3%.

In some embodiments, compositions may be formulated as a powder substantially free of water wherein the composition is reconstituted to a solution, a suspension, an ointment, or a cream just prior to use by the patient or a treating physician. Some embodiments may contain the active ingredients and other excipients, but are free of water. Of course, the active ingredient and/or one or more excipient may be hygroscopic and as such may contain small amount of water. Some embodiments contain no more than 0.1%, 0.5%, 1%, 2%, 3%, 4%, 5%, 6%, 7%, 8%, 9%, or 10% by weight of water in the composition.

The preparations may contain from approximately 0.0001% to about 5% for each of the one or more parasympathomimetic drugs and the one or more alpha agonists.

In one embodiment, the preparation comprises brimonidine and a parasympathomimetic drug. In one embodiment, the parasympathomimetic drug is pilocarpine. In another embodiment, the parasympathomimetic drug is carbachol. In another embodiment, the parasympathomimetic agent is phentermine. In another embodiment, the preparation comprises phentolamine and a parasympathomimetic drug.

In some preferred embodiments using brimonidine, the brimonidine concentration is approximately 0.20% or less. In other preferred embodiments using brimonidine, the brimonidine concentration is approximately 0.15% or less. In other preferred embodiments, the brimonidine concentration is approximately 0.10% or less. In another preferred embodiment, the brimonidine concentration is approximately 0.05% or less.

The one or more parasympathomimetic drugs and the one or more alpha agonists may be present in the pharmaceutical preparation as a pharmaceutically acceptable addition salt. Pharmaceutically acceptable salts are well known in the art and refer to the relatively non-toxic, inorganic and organic acid addition salts of the compound of the present invention. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In certain embodiments, at least one of the drugs is present in an amount lower than 75% of its effective dose for the purpose for which it is used when administered alone. For example, when pilocarpine is the drug which may be present in an amount lower than 75% of its dosage when used alone, then pilocarpine may be present in the preparation at more than about 3% or 4%. For example, carbachol can be 2.25% vs the normal effective dose of 3% and pilocarpine at 0.5-1.0% vs the normal effective dose of 2%.

When the alpha2 agonist present in the preparation is brimonidine, some embodiments may include about 0.3% or less, no more than 0.25%, no more than 0.2%, no more than 0.19%, no more than 0.18%, no more than 0.17%, no more than 0.16%, no more than 0.15%, no more than 0.14%, no more than 0.13%, no more than 0.12%, no more than 0.11%, no more than 0.1% brimonidine, no more than 0.09% brimonidine, nor more than 0.08% brimonidine, no more than 0.07% brimonidine, no more than 0.06 brimonidine, no more than 0.05% brimonidine, or its pharmaceutically acceptable salt.

When the alpha2 agonist present in the preparation is thymoxamine, some embodiments may include about 2% or less or its pharmaceutically acceptable salt.

When the alpha2 agonist present in the preparation is naphazoline, some embodiments may include about 0.2% or less, no more than 0.15%, no more than 0.125%, no more than 0.12%, no more than 0.11%, no more than 0.10%, no more than 0.09%, no more than 0.08%, no more than 0.07%, no more than 0.06%, no more than 0.05% naphazoline or its pharmaceutically acceptable salt. In some embodiments containing pilocarpine, or a pharmaceutically acceptable salt thereof, as the parasympathomimetic drug, the formulations may contain about 4% or less, 3% or less, no more than 2.8%, no more than 2.6%, no more than 2.5%, no more than 2.3%, no more than 2.0%, no more than 1.8%, no more than 1.6%, no more than 1.5%, no more than 1.2%, no more than 1%, no more than 0.9%, no more than 0.8%, no more than 0.7%, no more than 0.6%, no more than 0.5%, no more than 0.4%, no more than 0.3%, no more than 0.275%, no more than 0.25%, no more than 0.225%, no more than 0.2%, no more than 0.175%, no more than 0.15%, no more than 0.125%, no more than 0.1%, no more than 0.09%, no more than 0.08%, no more than 0.07%, no more than 0.06%, no more than 0.05%, no more than 0.04%, no more than 0.03%, no more than 0.02%, no more than 0.01%, no more than 0.005%, no more than 0.0025%, no more than 0.00125%, or no more than 0.001% of pilocarpine or its pharmaceutically acceptable salt.

When the parasympathomimetic drug present in the formulation is carbachol, or its pharmaceutically acceptable salt, some embodiments may contain about 5% or less, no more than 4.5%, no more than 4%, no more than 3.5%, no more than 3%, no more than 2.75%, no more than 2.5%, no more than 2.25%, no more than 2%, no more than 1.75%, no more than 1.5%, no more than 1.25%, no more than 1%, no more than 0.75%, no more than 0.5%, no more than 0.4%, no more than 0.3%, no more than 0.2%, or no more than 0.1% carbachol or its pharmaceutically acceptable salt.

Certain embodiments may contain phentolamine, or a pharmaceutically acceptable salt thereof, as the alpha antagonist. In those embodiments, the preparation may contain about 5% or less, no more than 4%, no more than 3.5%, no more than 3%, no more than 2.5%, no more than 2%, no more than 1.8%, no more than 1.6%, no more than 1.4%, no more than 1.2%, no more than 1%, no more than 0.9%, no more than 0.8%, no more than 0.7%, no more than 0.6%, no more than 0.5%, no more than 0.4%, no more than 0.3%, no more than 0.275%, no more than 0.25%, no more than 0.225%, no more than 0.2%, no more than 0.175%, no more than 0.15%, no more than 0.125%, no more than 0.1%, no more than 0.09%, no more than 0.08%, no more than 0.07%, no more than 0.06%, no more than 0.05%, no more than 0.04%, no more than 0.03%, no more than 0.02%, no more than 0.01%, no more than 0.005%, no more than 0.0025%, no more than 0.00125%, or no more than 0.001% of phentolamine or its pharmaceutically acceptable salt.

Unless otherwise specified elsewhere, the “%” in dosages in the preparations are intended to signify weight percentages.

When the “%” of a monomer in a co-polymer is specified, then that percentage is intended to mean mole (or repeat unit) percent. Thus, in copolymers, repeat units of each monomer are counted to calculate the total number of units of each monomer present in the co-polymer. For example, a co-polymer of two monomers containing on average (number average) three units of one monomer (say monomer A) for every seven units of another monomer (say monomer B) is said to contain 30% monomer A and 70% monomer B.

The pharmaceutical preparation which contains the one or more parasympathomimetic drugs and the one or more alpha agonists may be conveniently admixed with a non-toxic pharmaceutical organic carrier, or with a non-toxic pharmaceutical inorganic carrier. Typical pharmaceutically acceptable carriers are, for example, water, mixtures of water and water-miscible solvents such as lower alkanols or aralkanols, vegetable oils, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The pharmaceutical preparation may contain a buffering agent to maintain the pH in the therapeutically useful range of approximately 4.5 to 8.5. In certain embodiments, the pH is adjusted to about 5-8. In other embodiments, the pH is adjusted to about 6-7.5. In other embodiments, the pH is adjusted to about 7.3. Buffering agents used are those known to those skilled in the art, and, while not intending to be limiting, some examples are acetate, borate, carbonate, citrate, and phosphate buffers. In one embodiment of this invention, boric acid is the buffering agent.

The pharmaceutical preparations may contain one or more emulsifiers. As used herein, an “emulsifier” promotes the formation and/or stabilization of an emulsion. Suitable emulsifiers may be natural materials, finely divided solids, or synthetic materials. Natural emulsifying agents may be derived from either animal or vegetable sources. Those from animal sources include gelatin, egg yolk, casein, wool fat, or cholesterol. Those from vegetable sources include acacia, tragacanth, chondrus, or pectin. Vegetable sources specifically from cellulose derivatives include methyl cellulose and carboxymethyl cellulose to increase the viscosity. Finely divided emulsifiers include bentonite, magnesium hydroxide, aluminum hydroxide, or magnesium trisylicate. Synthetic agents include anionic, cationic or nonionic agents. Particularly useful emulsifiers are sodium lauryl sulfate, benzalkonium chloride or polyethylene glycol 400 monostearate, or any combinations thereof.

The pharmaceutical preparations may contain one or more thickeners. As used herein, a “thickener” refers to an agent that makes the preparation of the present invention dense or viscous in consistency. Suitable thickeners that can be used in the context of the present invention include, for example, non-ionic water-soluble polymers such as hydroxyethylcellulose (commercially available under the Trademark Natroso® 250 or 350), cationic water-soluble polymers such as Polyquat 37 (commercially available under the Trademark Synthalen® CN), fatty alcohols, fatty acids, anionic polymers, and their alkali salts and mixtures thereof.

The pharmaceutical preparations may contain one or more solubilizing agents. As used herein, the term “solubilizing agents” refers to those substances that enable solutes to dissolve. Representative examples of solubilizing agents that are usable in the context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN® and spans, e.g., TWEEN 80®. Other solubilizers that are usable for the preparations of the present invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

The pharmaceutical preparation may contain a mucoadhesive. As used herein, the term “mucoadhesive” means a natural or synthetic component, including macromolecules, polymers, and oligomers, or mixtures thereof, that can adhere to a subject's mucous membrane. Adhesion of mucoadhesives to the mucous membrane occurs primarily through noncovalent interactions, such as hydrogen bonding and Van der Waal forces. Examples of mucoadhesives for use in the embodiments disclosed herein include, but are not limited to, Carbopol®, pectin, alginic acid, alginate, chitosan, hyaluronic acid, polysorbates, such as polysorbate-20, -21, -40, -60, -61, -65, -80, -81, -85; poly(ethyleneglycol), such as PEG-7, -14, -16, -18, -55, -90, -100, -135, -180, -4, -240, -6,-8, -9, -10, -12, -20, or -32; oligosaccharides and polysaccharides, such as Tamarind seed polysaccharide, gellan, carrageenan, xanthan gum, gum Arabic, and dextran; cellulose esters and cellulose ethers; modified cellulose polymers, such as carboxymethylcellulose, hydroxyethylcellulose, hydroxypropyl methylcellulose, hydroxyethyl ethylcellulose; polyether polymers and oligomers, such as polyoxyethylene; condensation products of poly(ethyleneoxide) with various reactive hydrogen containing compounds having long hydrophobic chains (e.g. aliphatic chains of about 12 to 20 carbon atoms), for example, condensation products of poly(ethylene oxide) with fatty acids, fatty alcohols, fatty amides, polyhydric alcohols; polyether compounds, such as poly(methyl vinyl ether), polyoxypropylene of less than 10 repeating units; polyether compounds, such as block copolymers of ethylene oxide and propylene oxide; mixtures of block copolymers of ethylene oxide and propylene oxide with other excipients, for example poly(vinyl alcohol); polyacrylamide; hydrolyzed polyacrylamide; poly(vinyl pyrrolidone); poly(methacrylic acid); poly(acrylic acid) or crosslinked polyacrylic acid, such as Carbomer®, i.e., a homopolymer of acrylic acid crosslinked with either an allyl ether of pentaerythritol, an allyl ether of sucrose, or an allyl ether of propylene. In certain embodiments the mucoadhesive is a polysaccharide. One polysaccharide which is particularly useful as a mucoadhesive in the embodiments disclosed herein is Tamarind seed polysaccharide, which is a galactoxyloglucan that is extracted from the seed kernel of Tamarindus Indica, and can be purchased from TCI America of Portland, Oreg.

The pharmaceutical preparations may contain a tonicity agent to adjust the preparation to the desired isotonic range. Tonicity agents are known to those skilled in the ophthalmic art, and, while not intending to be limiting, some examples include glycerin, mannitol, sorbitol, sodium chloride, and other electrolytes. In one embodiment, the tonicity agent is glycerin. In another embodiment, the tonicity agent is a chloride salt. In some embodiments, the ionic content adjusted to about 0.5% to about 1.8%, expressed as sodium chloride equivalents. In these embodiments, the preparation may, in addition to tonicity adjusting ingredients, comprise an opthalmically acceptable, water-soluble, non-ionic synthetic polymer having a molecular weight within the range of 300 to 250,000, and a non-charged, non-ionic tonicity adjusting agent.

The exact percentage of the non-ionic synthetic polymer used in the solution will depend on the molecular weight of the selected polymer. However, it is intended that, absent the presence of additional viscosity building agents, the ophthalmic solution will generally have a viscosity between about 1 to about 10 cps. In certain embodiments, the ophthalmic solution has a viscosity of about 2 cps to about 8 cps at 23° C. For example, polyvinyl alcohol and polyethylene glycol are among those non-ionic polymeric substances that may be incorporated into the preparations of the present invention. When polyvinyl alcohol is added to the solution, it will be present in a concentration of from about 0.1% to about 5%, or even from about 0.25% to about 2%, whereas when polyethylene glycol is used it will comprise from about 0.25% to about 3% of the solution. Such polymers are commercially available and their composition well known to those skilled in the art.

The pharmaceutical preparation may contain a preservative. Preservatives are used to prevent bacterial contamination in multiple-use ophthalmic preparations, and, while not intending to be limiting, examples include benzalkonium chloride, stabilized oxychloro complexes (otherwise known as Purite®), phenylmercuric acetate, chlorobutanol, benzyl alcohol, parabens, and thimerosal. In some embodiments, the preservative is Purite®.

The pharmaceutical preparation may contain a chelating agent to enhance preservative effectiveness. Suitable chelating agents are those known in the art, and, while not intending to be limiting, edetate salts like edetate disodium, edetate calcium disodium, edetate sodium, edetate trisodium, and edetate dipotassium are examples of useful preservatives.

The pharmaceutical preparations may be formulated as a sustained release formulation where the active ingredients are released over several hours. For example, a stable fluid preparation for the sustained release preparation may comprise a synthetic polymer comprising both hydrophilic and hydrophobic components such that the active ingredients become encapsulated or dispersed in micellar droplets.

The polymer may be a homopolymer of a monomer containing a pendent hydrophilic group such as an acid group, or it may be a copolymer of different monomers, some or all of which contain pendent hydrophilic groups such as an acid group. The monomers may be vinyl monomers. The co-polymer may contain about 10% or more of the monomer containing the hydrophilic pendent group. In one embodiment, more than 25% by weight of the monomers contain a hydrophilic pendent group. In another embodiment, more than 40% by weight of the monomers contain a hydrophilic pendent group. In certain embodiments, 10-100% by weight of the monomers contain a hydrophilic pendent group and 0-90% of the monomers are hydrophobic monomers. In other embodiments, 25-100% by weight of the monomers contain a hydrophilic pendent group and 0-75% of the monomers are hydrophobic monomers. In further embodiments, 40-100% by weight of the monomers contain a hydrophilic pendent group, and 0-60% of the monomers are hydrophobic monomers.

The particular choice of monomers are made with regard to the desired solubility or dispersability of the polymer, the desired release pattern and other properties required of the particular formulation. Although the polymers used in the present preparations are generally free of cross-linking agent and comprise both hydrophilic monomers and hydrophobic monomers, cross-linking may be used as additional control of the properties of the polymer. For instance, one could include a small amount of a trifunctional cross-linkable monomer in the monomer mixture from which the polymer is made. The amount of cross-linkable monomer is generally small, for instance 1-15% by weight, or 1-10% by weight. In certain embodiments, the polymers may comprise from 10 to 75% hydrophilic monomers and from 20 to 80% hydrophobic monomers. In other embodiments, the polymers may comprise from 10 to 55% hydrophilic monomers and from 30 to 60% hydrophobic monomers.

Suitable hydrophilic monomers include monomeric acids, such as acrylic, methacrylic, itaconic, crotonic, vinyl sulfonic, maleic, angelic, oleic, or α-chloro-acrylic acid or sulfoethyl-methacrylate and vinyl pyrrolidone. Naturally dicarboxylic acids such as maleic acid may be introduced in the form of the anhydride.

Suitable hydrophobic monomers include alkyl acrylates, alkyl methacrylates, vinyl ethers, acrylonitrile, hydroxymethacrylate, styrene and vinyl acetate. The alkyl groups in alkylacrylates and alkylmethacrylates usually contain 1 to 4 carbon atoms, e.g. ethyl, methyl or butyl, but longer chain groups containing up to, say, 18 carbon atoms, e.g., lauryl, can be used. In particular when hydrophobic monomer is present, at least part of it can be a plasticizing monomer in a proportion of 5% to 20% by weight. In certain embodiments, the plasticizing monomer makes about 10% of the polymer. Suitable plasticizing monomers are long chain esters of acrylic or methacrylic acid, e.g. ethyl hexyl acrylate.

In certain embodiments, the polymer is a copolymer of hydrophilic monomers selected from acrylic acid, vinyl pyrrolidone, methacrylic acid and maleic anhydride and hydrophobic monomers selected from methyl methacrylate, butyl methacrylate, lauryl methacrylate, methylacrylate, 2-ethyl-hexylacrylate and styrene. In another embodiment, the polymer may include acrylic acid with or without vinyl pyrrolidone. In certain embodiments, the polymer may contain from 20 to 55% acrylic acid.

### Example 1

Ophthalmic solution in 100 ml

| Ingredient | Amount | |
|---|---|---|
| Brimonidine or its pharma- | 0.1, 0.15, 0.2, or 0.25 g | |
| ceutically acceptable salt | | |
| Carbachol or its pharma- | 5, 4.5, 4, 3.5, 3, 2.75, 2.5, 2.25, 2, 1.75, | |
| ceutically acceptable salt | 1.5, 1.25, 1, 0.75, or 0.5 g | |
| Sodium Chloride | 0.4 | g |
| D-Glucose | 0.04 | g |
| Sterilized refined water | Balance | |
| Total | 100 | ml |

The ingredients in this example are prepared in a usual manner into a sterilized preparation as an ophthalmic solution, adjusting, if necessary, the pH to about 7.3. This example provides for sixty different ophthalmic preparations.

### Example 2

Ophthalmic solution in 100 ml

| Ingredient | Amount | |
|---|---|---|
| Brimonidine or its pharmaceutically | 0.1, 0.15, 0.2, or 0.25 g | |
| acceptable salt | | |
| Pilocarpine or its pharmaceutically | 3, 2.8, 2.6, 2.5, 2.3, 2.0, 1.8, 1.6, 1.5, | |
| acceptable salt | 1.2, 1, 0.9, 0.8, 0.7, 0.6, 0.5, 0.4, 0.3, | |
| | 0.2, or 0.1 g | |
| Sodium Chloride | 0.4 | g |
| D-Glucose | 0.04 | g |
| Sterilized refined water | Balance | |
| Total | 100 | ml |

The ingredients in this example are prepared in a usual manner into a sterilized preparation as an ophthalmic solution, adjusting, if necessary, the pH to 7.3 using a buffer solution. This example provides for eighty different ophthalmic preparations.

### Example 3

Ophthalmic solution in 100 ml

| Ingredient | Amount | |
|---|---|---|
| Brimonidine or its pharmaceutically | 0.1, 0.15, 0.2, or 0.25 g | |
| acceptable salt | | |
| Phentolamine or its pharmaceutically | 0.1, 0.2, or 0.4 g | |
| acceptable salt | | |
| Sodium Chloride | 0.4 | g |
| D-Glucose | 0.04 | g |
| Sterilized refined water | Balance | |
| Total | 100 | ml |

The ingredients in this example are prepared in a usual manner into a sterilized preparation as an ophthalmic solution, adjusting, if necessary, the pH to 7.3 using a buffer solution. This example provides for twelve different ophthalmic preparations.

### Example 4

The effect of pilocarpine alone or in combination with brimonidine on near visual acuity (VA) for patients suffering from presbyopia was evaluated. Initially, 10 patients were selected for preliminary evaluation. Each patient was administered with one drop of a formulation containing 0.25%, 0.5%, or 1.0% pilocarpine with or without one drop of a formulation containing 0.2% brimonidine. The six dosages that were initially tested are shown in Table 1.

TABLE 1
Dosages Tested
0.25% Pilocarpine
0.50% Pilocarpine
1.0% Pilocarpine
0.25% Pilocarpine & 0.2%
Brimonidine
0.5% Pilocarpine & 0.2%
Brimonidine
1.0% Pilocarpine & 0.2%
Brimonidine

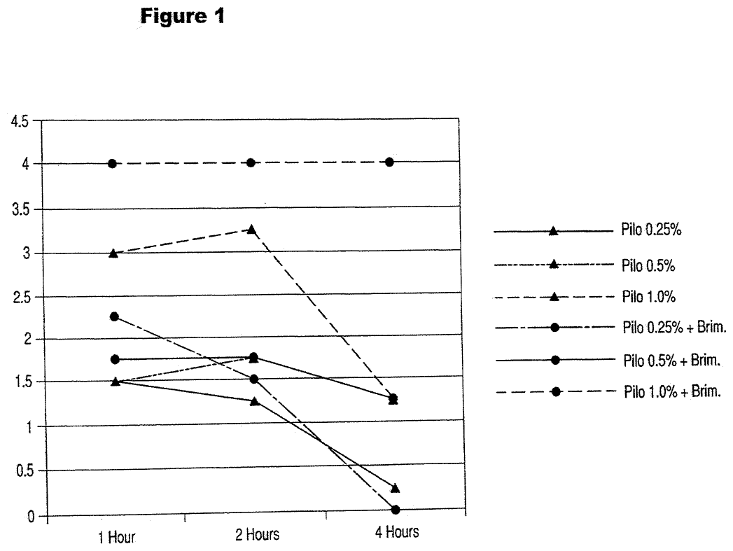

FIG. 1 shows change in visual acuity at 1 hr, 2 hrs, and 4 hrs after administration of one of the six dosages described above. Although some patients complained of burning symptoms on their eyes, it should be noted that the formulations were not optimized for patient comfort. All six dosages provided some (varying) initial improvement in visual acuity. However, the effect of formulations containing pilocarpine alone appears to wear out fairly quickly whereas it takes longer time for effects of formulations containing both drugs to wear out.

To further understand the effectiveness of the formulation containing 0.5% pilocarpine and 0.2% brimonidine, applicant conducted a double-blind, randomized clinical trial. Forty patients suffering from presbyopia were recruited. The patients were randomly divided into two arms: the active drug arm and the placebo arm. Prior to receiving the treatment, the visual acuity of each patient was measured. On day 1 of the trial, patients enrolled in the active drug arm received one drop containing 0.5%, pilocarpine and one drop containing 0.2% brimonidine. The skilled artisan would recognize that the two drugs can be formulated as a composition containing both drugs and applied the desired number of drops of the composition to the eye such that both drugs are delivered to the eye simultaneously. Patients enrolled in placebo arm received two drop of placebo. Patients' responses to the treatment were examined by measuring the visual acuity of each patient at 1, 2, 4, 8, and 10 hrs. after treatment. The treatment was repeated for seven days, each time administering the specified amount and examining patients' responses at 1, 2, 4, 8, and 10 hrs. after treatment by measuring their visual acuity. Table 2, parts 1-6 lists patients' visual acuities, measured prior to the treatment and at 1, 2, 4, 8, and 10 hrs. after treatment for days 1 through 7. Although some patients complained of burning symptoms on their eyes, it should be noted that the formulations were not optimized for patient comfort.

TABLE 2
Part 1

| Pilocarpine 0.5% Plus Brimonidine 0.2% Eye Drops Vs. Placebo to Reduce The Need for Reading | | |
|---|---|---|
| Glasses (X 7 DAYS) | | |
| | Day 1 | Day 2 |

| | Pre-treatment | | | Near | Near | Near | Near | Near | Near | Near | Near | Near | Near |
|---|---|---|---|---|---|---|---|---|---|---|---|---|---|
| | | | Near | Va(j) | Va(j) | Va(j) | Va(j) | Va(j) | Va(j) | Va(j) | Va(j) | Va(j) | Va(j) |
| | Age | Sex | VA(j) | 1 hr | 2 hr | 4 hr | 8 hr | 10 hr | 1 hr | 2 hr | 4 hr | 8 hr | 10 hr |
| Pilocarpine 0.56% | 55 | M | 8 | 3 | 5 | 6 | 8 | 8 | 3 | 3 | 5 | 8 | 8 |
| Plus Brimonidine | 50 | F | 5 | 2 | 2 | 2 | 3 | 5 | 2 | 2 | 2 | 3 | 5 |
| 0.2% Eye Drops | 55 | F | 8 | 5 | 5 | 6 | 7 | 8 | 4 | 5 | 5 | 6 | 8 |
| | 48 | F | 8 | 3 | 2 | 5 | 8 | 8 | 3 | 3 | 5 | 8 | 8 |
| | 49 | F | 6 | 3 | 5 | 5 | 5 | 6 | 4 | 5 | 5 | 5 | 6 |
| | 48 | M | 3 | 1 | 1 | 1 | 2 | 3 | 1 | 1 | 1 | 1 | 2 |
| | 52 | M | 8 | 5 | 5 | 6 | 8 | 8 | 6 | 5 | 8 | 8 | 8 |
| | 55 | F | 10 | 8 | 8 | 10 | 10 | 10 | 8 | 8 | 10 | 10 | 10 |
| | 47 | M | 6 | 2 | 3 | 5 | 5 | 6 | 2 | 3 | 5 | 5 | 6 |
| | 45 | F | 3 | 1 | 1 | 1 | 2 | 3 | 1 | 1 | 1 | 2 | 3 |
| | 47 | F | 5 | 2 | 2 | 3 | 5 | 5 | 2 | 2 | 3 | 4 | 5 |
| | 54 | M | 8 | 3 | 3 | 5 | 7 | 8 | 3 | 3 | 5 | 5 | 8 |
| | 49 | F | 6 | 4 | 4 | 5 | 6 | 6 | 4 | 5 | 5 | 6 | 6 |
| | 53 | M | 8 | 4 | 4 | 4 | 7 | 8 | 4 | 4 | 5 | 8 | 8 |
| | 53 | M | 7 | 5 | 5 | 6 | 7 | 7 | 5 | 5 | 6 | 7 | 7 |
| | 47 | F | 5 | 2 | 2 | 2 | 3 | 5 | 1 | 2 | 3 | 5 | 5 |
| | 45 | M | 5 | 1 | 1 | 2 | 3 | 5 | 1 | 1 | 2 | 3 | 5 |
| | 48 | M | 5 | 2 | 2 | 3 | 5 | 5 | 2 | 2 | 5 | 5 | 5 |
| | 53 | F | 6 | 2 | 2 | 5 | 6 | 6 | 2 | 2 | 5 | 6 | 6 |
| | 45 | M | 3 | 1 | 1 | 1 | 2 | 3 | 1 | 1 | 1 | 3 | 3 |
| | Sum | | 123 | 59 | 63 | 83 | 109 | 123 | 59 | 64 | 87 | 102 | 122 |
| | AVERAGE | | 6.15 | 2.95 | 3.15 | 4.15 | 5.45 | 6.15 | 2.95 | 3.2 | 4.35 | 5.4 | 6.1 |

Part 2

| Pilocarpine 0.5% Plus Brimonidine 0.2% Eye Drops Vs. Placebo to Reduce The Need for Reading | | |
|---|---|---|
| Glasses (X 7 DAYS) | | |
| | Day 1 | Day 2 |

| | Pre-treatment | | | Near | Near | Near | Near | Near | Near | Near | Near | Near | Near |
|---|---|---|---|---|---|---|---|---|---|---|---|---|---|
| | | | Near | Va(j) | Va(j) | Va(j) | Va(j) | Va(j) | Va(j) | Va(j) | Va(j) | Va(j) | Va(j) |
| | Age | Sex | VA(j) | 1 hr | 2 hr | 4 hr | 8 hr | 10 hr | 1 hr | 2 hr | 4 hr | 8 hr | 10 hr |
| Placebo Eye Drops | 49 | M | 6 | 6 | 6 | 6 | 6 | 6 | 6 | 6 | 6 | 6 | 6 |
| | 51 | M | 5 | 5 | 5 | 5 | 5 | 5 | 5 | 5 | 5 | 5 | 5 |
| | 50 | M | 5 | 4 | 5 | 5 | 5 | 5 | 4 | 5 | 5 | 5 | 5 |
| | 54 | F | 8 | 8 | 8 | 8 | 8 | 9 | 7 | 8 | 8 | 8 | 8 |
| | 49 | F | 5 | 4 | 5 | 5 | 5 | 5 | 4 | 5 | 5 | 5 | 5 |
| | 52 | M | 6 | 6 | 6 | 6 | 6 | 6 | 6 | 6 | 6 | 6 | 6 |
| | 47 | M | 5 | 5 | 5 | 5 | 5 | 5 | 5 | 5 | 5 | 5 | 5 |
| | 45 | F | 3 | 3 | 3 | 3 | 3 | 3 | 2 | 3 | 3 | 3 | 3 |
| | 54 | M | 8 | 8 | 8 | 8 | 8 | 8 | 8 | 8 | 8 | 8 | 8 |
| | 53 | F | 8 | 8 | 8 | 8 | 8 | 8 | 8 | 8 | 8 | 8 | 8 |
| | 49 | F | 6 | 5 | 6 | 6 | 6 | 6 | 6 | 6 | 6 | 6 | 6 |
| | 55 | F | 8 | 6 | 8 | 8 | 8 | 8 | 8 | 8 | 8 | 8 | 8 |
| | 52 | M | 8 | 8 | 8 | 8 | 8 | 8 | 8 | 8 | 8 | 8 | 8 |
| | 46 | F | 6 | 5 | 6 | 6 | 6 | 6 | 5 | 5 | 6 | 6 | 6 |
| | 48 | F | 6 | 6 | 6 | 6 | 6 | 6 | 6 | 6 | 6 | 6 | 6 |
| | 50 | M | 5 | 5 | 5 | 5 | 5 | 5 | 5 | 5 | 5 | 5 | 5 |
| | 50 | M | 6 | 6 | 6 | 6 | 6 | 6 | 6 | 6 | 6 | 6 | 6 |
| | 47 | F | 5 | 3 | 5 | 5 | 5 | 5 | 3 | 3 | 5 | 5 | 5 |
| | 54 | M | 10 | 10 | 10 | 10 | 10 | 10 | 10 | 10 | 10 | 10 | 10 |
| | 45 | F | 3 | 2 | 3 | 3 | 3 | 3 | 2 | 3 | 3 | 3 | 3 |
| | SUM | | 122 | 113 | 122 | 122 | 122 | 123 | 114 | 119 | 122 | 122 | 122 |
| | AVERAGE | | 6.1 | 5.65 | 6.1 | 6.1 | 6.1 | 6.15 | 5.7 | 5.95 | 6.1 | 6.1 | 6.1 |

Part 3

| | | Pilocarpine 0.5% Plus Brimonidine 0.2% Eye Drops Vs. Placebo to Reduce The Need For Reading | | | | | | | | | | | | |
|---|---|---|---|---|---|---|---|---|---|---|---|---|---|---|
| | Glasses (X 7 DAYS) | | | | | | | | | | | | | |
| | | | | | Day 3 | | | | | Day 4 | | | | |
| | Pre-treatment | | | | Near | Near | Near | Near | Near | Near | Near | Near | Near | Near |
| | | | Near | — | Va(j) | Va(j) | Va(j) | Va(j) | Va(j) | Va(j) | Va(j) | Va(j) | Va(j) | Va(j) |
| | Age | Sex | VA(j) | — | 1 hr | 2 hr | 4 hr | 8 hr | 10 hr | 1 hr | 2 hr | 4 hr | 8 hr | 10 hr |
| Pilocarpine 0.56% | 55 | M | 6 | — | 2 | 3 | 5 | 8 | 8 | 2 | 3 | 5 | 8 | 8 |
| Plus Brimonidine | 50 | F | 5 | — | 2 | 2 | 2 | 3 | 5 | 2 | 2 | 2 | 3 | 5 |
| 0.2% Eye Drops | 55 | F | 5 | — | 6 | 6 | 6 | 6 | 8 | 6 | 6 | 6 | 6 | 8 |
| | 48 | F | 8 | — | 3 | 3 | 5 | 8 | 8 | 3 | 3 | 5 | 8 | 8 |
| | 49 | F | 5 | — | 4 | 5 | 5 | 5 | 6 | 4 | 5 | 5 | 6 | 6 |
| | 48 | M | 6 | — | 1 | 1 | 1 | 2 | 3 | 1 | 1 | 2 | 2 | 3 |
| | 52 | M | 5 | — | 5 | 6 | 8 | 8 | 8 | 5 | 5 | 8 | 8 | 10 |
| | 55 | F | 3 | — | 8 | 8 | 10 | 10 | 10 | 8 | 8 | 10 | 10 | 10 |
| | 47 | M | 8 | — | 2 | 2 | 3 | 5 | 6 | 2 | 2 | 3 | 5 | 6 |
| | 45 | F | 8 | — | 1 | 1 | 1 | 2 | 3 | 1 | 1 | 1 | 2 | 3 |
| | 47 | F | 6 | — | 3 | 3 | 3 | 4 | 5 | 2 | 2 | 3 | 4 | 5 |
| | 54 | M | 8 | — | 3 | 3 | 5 | 6 | 8 | 3 | 3 | 6 | 6 | 8 |
| | 49 | F | 8 | — | 3 | 4 | 4 | 6 | 6 | 3 | 4 | 4 | 5 | 6 |
| | 53 | M | 6 | — | 4 | 4 | 6 | 8 | 8 | 4 | 4 | 6 | 8 | 8 |
| | 53 | M | 6 | — | 5 | 5 | 6 | 7 | 7 | 5 | 5 | 7 | 7 | 7 |
| | 47 | F | 5 | — | 1 | 1 | 3 | 5 | 5 | 1 | 1 | 2 | 5 | 5 |
| | 45 | M | 6 | — | 1 | 1 | 2 | 3 | 5 | 1 | 1 | 3 | 3 | 5 |
| | 48 | M | 5 | — | 2 | 2 | 3 | 3 | 5 | 2 | 2 | 3 | 5 | 5 |
| | 53 | F | 10 | — | 2 | 2 | 5 | 6 | 6 | 2 | 2 | 5 | 6 | 6 |
| | 45 | M | 3 | — | 1 | 1 | 1 | 3 | 3 | 1 | 1 | 1 | 2 | 3 |
| | SUM | | 122 | — | 59 | 63 | 84 | 106 | 123 | 58 | 61 | 87 | 109 | 125 |
| | AVERAGE | | 6.1 | — | 2.95 | 3.15 | 4.2 | 5.3 | 6.15 | 2.9 | 3.05 | 4.35 | 5.45 | 6.25 |
| | | | | | | | | | | Day 5 | | | | |
| | | | | | | | | | | Near | Near | Near | Near | Near |
| | | | | | | | | | | Va(j) | Va(j) | Va(j) | Va(j) | Va(j) |
| | | | | | | | | | | 1 hr | 2 hr | 4 hr | 8 hr | 10 hr |
| | | | | | | | | | | 3 | 3 | 5 | 8 | 8 |
| | | | | | | | | | | 2 | 2 | 3 | 3 | 5 |
| | | | | | | | | | | 6 | 6 | 6 | 6 | 8 |
| | | | | | | | | | | 3 | 3 | 5 | 8 | 8 |
| | | | | | | | | | | 4 | 5 | 5 | 6 | 6 |
| | | | | | | | | | | 1 | 1 | 1 | 2 | 3 |
| | | | | | | | | | | 5 | 8 | 8 | 8 | 8 |
| | | | | | | | | | | 8 | 8 | 10 | 10 | 10 |
| | | | | | | | | | | 2 | 2 | 3 | 5 | 6 |
| | | | | | | | | | | 1 | 1 | 1 | 2 | 3 |
| | | | | | | | | | | 2 | 2 | 3 | 4 | 5 |
| | | | | | | | | | | 3 | 3 | 3 | 6 | 8 |
| | | | | | | | | | | 3 | 3 | 4 | 4 | 6 |
| | | | | | | | | | | 4 | 4 | 7 | 8 | 8 |
| | | | | | | | | | | 4 | 4 | 5 | 6 | 7 |
| | | | | | | | | | | 1 | 1 | 3 | 5 | 5 |
| | | | | | | | | | | 1 | 1 | 3 | 3 | 5 |
| | | | | | | | | | | 2 | 2 | 3 | 5 | 5 |
| | | | | | | | | | | 2 | 2 | 5 | 6 | 6 |
| | | | | | | | | | | 1 | 1 | 1 | 2 | 3 |
| | | | | | | | | | | 58 | 62 | 84 | 107 | 123 |
| | | | | | | | | | | 2.9 | 3.1 | 4.2 | 5.35 | 6.15 |

Part 4

| | | Pilocarpine 0.5% Plus Brimonidine 0.2% Eye Drops Vs. Placebo to Reduce The Need For Reading | | | | | | | | | | | | |
|---|---|---|---|---|---|---|---|---|---|---|---|---|---|---|
| | | Glasses (X 7 DAYS) | | | | | | | | | | | | |
| | | | | | Day 3 | | | | | Day 4 | | | | |
| | Pre-treatment | | | | Near | Near | Near | Near | Near | Near | Near | Near | Near | Near |
| | | | Near | — | Va(j) | Va(j) | Va(j) | Va(j) | Va(j) | Va(j) | Va(j) | Va(j) | Va(j) | Va(j) |
| | Age | Sex | VA(j) | — | 1 hr | 2 hr | 4 hr | 8 hr | 10 hr | 1 hr | 2 hr | 4 hr | 8 hr | 10 hr |
| Placebo Eye Drops | 49 | M | 6 | — | 6 | 6 | 6 | 6 | 6 | 6 | 6 | 6 | 6 | 6 |
| | 51 | M | 5 | — | 5 | 5 | 5 | 5 | 5 | 5 | 5 | 5 | 5 | 5 |
| | 50 | M | 5 | — | 4 | 5 | 5 | 5 | 5 | 4 | 5 | 5 | 5 | 5 |
| | 54 | F | 8 | — | 8 | 8 | 8 | 8 | 8 | 8 | 8 | 8 | 8 | 8 |
| | 49 | F | 5 | — | 4 | 5 | 5 | 5 | 5 | 4 | 5 | 5 | 5 | 5 |
| | 52 | M | 6 | — | 6 | 6 | 6 | 6 | 6 | 6 | 6 | 6 | 6 | 6 |
| | 47 | M | 5 | — | 5 | 5 | 5 | 5 | 5 | 5 | 5 | 5 | 5 | 5 |
| | 45 | F | 3 | — | 2 | 3 | 3 | 3 | 3 | 2 | 3 | 3 | 3 | 3 |
| | 54 | M | 8 | — | 8 | 8 | 8 | 8 | 8 | 8 | 8 | 8 | 8 | 8 |
| | 53 | F | 8 | — | 7 | 8 | 8 | 8 | 8 | 7 | 8 | 8 | 8 | 8 |
| | 49 | F | 6 | — | 6 | 6 | 6 | 6 | 6 | 6 | 6 | 6 | 6 | 6 |
| | 55 | F | 8 | — | 8 | 8 | 8 | 8 | 8 | 8 | 8 | 8 | 8 | 8 |
| | 52 | M | 8 | — | 8 | 8 | 8 | 8 | 8 | 8 | 8 | 8 | 8 | 8 |
| | 46 | F | 6 | — | 5 | 6 | 6 | 6 | 6 | 5 | 6 | 6 | 6 | 6 |
| | 48 | F | 6 | — | 6 | 6 | 6 | 6 | 6 | 6 | 6 | 6 | 6 | 6 |
| | 50 | M | 5 | — | 5 | 5 | 5 | 5 | 5 | 5 | 5 | 5 | 5 | 5 |
| | 50 | M | 6 | — | 6 | 6 | 6 | 6 | 6 | 6 | 6 | 6 | 6 | 6 |
| | 47 | F | 5 | — | 3 | 5 | 5 | 5 | 5 | 3 | 5 | 5 | 5 | 5 |
| | 54 | M | 10 | — | 10 | 10 | 10 | 10 | 10 | 10 | 10 | 10 | 10 | 10 |
| | 45 | F | 3 | — | 2 | 3 | 3 | 3 | 3 | 2 | 3 | 3 | 3 | 3 |
| | SUM | | 122 | — | 114 | 122 | 122 | 122 | 122 | 114 | 122 | 122 | 122 | 122 |
| | AVERAGE | | 6.1 | — | 5.7 | 6.1 | 6.1 | 6.1 | 6.1 | 5.7 | 6.1 | 6.1 | 6.1 | 6.1 |
| | | | | | | | | | | Day 5 | | | | |
| | | | | | | | | | | Near | Near | Near | Near | Near |
| | | | | | | | | | | Va(j) | Va(j) | Va(j) | Va(j) | Va(j) |
| | | | | | | | | | | 1 hr | 2 hr | 4 hr | 8 hr | 10 hr |
| | | | | | | | | | | 6 | 6 | 6 | 6 | 6 |
| | | | | | | | | | | 4 | 5 | 5 | 5 | 5 |
| | | | | | | | | | | 4 | 5 | 5 | 5 | 5 |
| | | | | | | | | | | 8 | 8 | 8 | 8 | 8 |
| | | | | | | | | | | 4 | 5 | 5 | 5 | 5 |
| | | | | | | | | | | 6 | 6 | 6 | 6 | 6 |
| | | | | | | | | | | 5 | 5 | 5 | 5 | 5 |
| | | | | | | | | | | 2 | 3 | 3 | 3 | 3 |
| | | | | | | | | | | 8 | 8 | 8 | 8 | 8 |
| | | | | | | | | | | 7 | 8 | 8 | 8 | 8 |
| | | | | | | | | | | 6 | 6 | 6 | 6 | 6 |
| | | | | | | | | | | 8 | 8 | 8 | 8 | 8 |
| | | | | | | | | | | 8 | 8 | 8 | 8 | 8 |
| | | | | | | | | | | 6 | 6 | 6 | 6 | 6 |
| | | | | | | | | | | 6 | 6 | 6 | 6 | 6 |
| | | | | | | | | | | 5 | 5 | 5 | 5 | 5 |
| | | | | | | | | | | 6 | 6 | 6 | 6 | 6 |
| | | | | | | | | | | 3 | 5 | 5 | 5 | 5 |
| | | | | | | | | | | 10 | 10 | 10 | 10 | 10 |
| | | | | | | | | | | 2 | 3 | 3 | 3 | 3 |
| | | | | | | | | | | 113 | 122 | 122 | 122 | 122 |
| | | | | | | | | | | 5.65 | 6.1 | 6.1 | 6.1 | 6.1 |
| | | | | | | | | | | | 6.1 | 6.1 | 6.1 | |

Part 5

| | | Pilocarpine 0.5% Plus Brimonidine 0.2% Eye Drops Vs. Placebo to Reduce The Need For Reading | | | | | | | | | | | | |
|---|---|---|---|---|---|---|---|---|---|---|---|---|---|---|
| | | Glasses (X 7 DAYS) | | | | | | | | | | | | |
| | | | | | Day 6 | | | | | Day 7 | | | | |
| | Pre-treatment | | | | Near | Near | Near | Near | Near | Near | Near | Near | Near | Near |
| | | | Near | — | Va(j) | Va(j) | Va(j) | Va(j) | Va(j) | Va(j) | Va(j) | Va(j) | Va(j) | Va(j) |
| | Age | Sex | VA(j) | — | 1 hr | 2 hr | 4 hr | 8 hr | 10 hr | 1 hr | 2 hr | 4 hr | 8 hr | 10 hr |
| Pilocarpine 0.56% | 55 | M | 6 | — | 3 | 3 | 5 | 8 | 8 | 2 | 3 | 5 | 8 | 8 |
| Plus Brimonidine | 50 | F | 5 | — | 2 | 2 | 2 | 3 | 5 | 2 | 2 | 2 | 3 | 5 |
| 0.2% Eye Drops | 55 | F | 5 | — | 5 | 5 | 6 | 7 | 8 | 5 | 6 | 6 | 7 | 8 |
| | 48 | F | 8 | — | 3 | 3 | 5 | 8 | 8 | 3 | 3 | 5 | 8 | 8 |
| | 49 | F | 5 | — | 4 | 5 | 6 | 6 | 6 | 4 | 5 | 5 | 6 | 6 |
| | 48 | M | 6 | — | 1 | 1 | 2 | 3 | 3 | 1 | 1 | 3 | 3 | 3 |
| | 52 | M | 5 | — | 5 | 5 | 7 | 7 | 8 | 5 | 5 | 7 | 8 | 8 |
| | 55 | F | 3 | — | 8 | 9 | 10 | 9 | 10 | 8 | 8 | 9 | 9 | 10 |
| | 47 | M | 8 | — | 2 | 2 | 3 | 6 | 6 | 2 | 2 | 3 | 6 | 6 |
| | 45 | F | 8 | — | 1 | 1 | 1 | 2 | 3 | 1 | 1 | 1 | 2 | 3 |
| | 47 | F | 6 | — | 2 | 2 | 3 | 5 | 5 | 2 | 3 | 3 | 5 | 5 |
| | 54 | M | 8 | — | 3 | 3 | 5 | 8 | 8 | 3 | 3 | 6 | 8 | 8 |
| | 49 | F | 8 | — | 3 | 3 | 4 | 6 | 6 | 3 | 3 | 4 | 6 | 6 |
| | 53 | M | 6 | — | 3 | 4 | 7 | 8 | 8 | 3 | 4 | 6 | 7 | 8 |
| | 53 | M | 6 | — | 4 | 4 | 6 | 7 | 7 | 4 | 4 | 7 | 7 | 7 |
| | 47 | F | 5 | — | 2 | 2 | 3 | 3 | 5 | 1 | 2 | 3 | 5 | 5 |
| | 45 | M | 6 | — | 1 | 1 | 2 | 5 | 5 | 1 | 1 | 3 | 5 | 5 |
| | 48 | M | 5 | — | 2 | 2 | 3 | 5 | 5 | 2 | 2 | 3 | 5 | 5 |
| | 53 | F | 10 | — | 2 | 2 | 5 | 6 | 6 | 2 | 2 | 5 | 6 | 6 |
| | 45 | M | 3 | — | 1 | 1 | 1 | 2 | 3 | 1 | 1 | 1 | 2 | 3 |
| | Sum | | 122 | — | 57 | 61 | 86 | 114 | 123 | 55 | 61 | 87 | 116 | 123 |
| | AVERAGE | | 6.1 | — | 2.85 | 3.05 | 4.3 | 5.7 | 6.15 | 17.5 | 3.05 | 4.35 | 5.8 | 6.15 |
| | | | | | | | | | | Averages | | | | |
| | | | | | | | | | | 1 hr = | 2 hr = | 4 hr = | 8 hr = | 10 hr = |
| | | | | | | | | | | Average | Average | Average | Average | Average |
| | | | | | | | | | | Near | Near | Near | Near | Near |
| | | | | | | | | | | VA(j) | VA(j) | VA(j) | VA(j) | VA(j) |
| | | | | | | | | | | 2.57 | 3.29 | 5.14 | 8 | 8 |
| | | | | | | | | | | 2 | 2 | 2.14 | 3 | 5 |
| | | | | | | | | | | 5.29 | 5.57 | 5.86 | 6.43 | 8 |
| | | | | | | | | | | 3 | 2.86 | 5 | 8 | 8 |
| | | | | | | | | | | 3.86 | 5 | 5.14 | 5.57 | 6 |
| | | | | | | | | | | 1 | 1 | 1.57 | 2.14 | 2.86 |
| | | | | | | | | | | 5.14 | 5.71 | 7.43 | 7.86 | 8.29 |
| | | | | | | | | | | 8 | 8.14 | 9.86 | 9.71 | 10 |
| | | | | | | | | | | 2 | 2.29 | 3.357 | 5.29 | 6 |
| | | | | | | | | | | 1 | 1 | 1 | 2 | 3 |
| | | | | | | | | | | 2.14 | 2.43 | 5 | 4.43 | 5 |
| | | | | | | | | | | 3 | 3 | 5 | 6.97 | 8 |
| | | | | | | | | | | 3.29 | 3.71 | 4.29 | 5.57 | 6 |
| | | | | | | | | | | 3.71 | 4 | 5.86 | 7.71 | 8 |
| | | | | | | | | | | 4.57 | 4.57 | 6.14 | 6.86 | 7 |
| | | | | | | | | | | 1.29 | 1.57 | 2.71 | 4.14 | 5 |
| | | | | | | | | | | 1 | 1 | 2.43 | 3.57 | 5 |
| | | | | | | | | | | 2 | 2 | 3.29 | 4.71 | 5 |
| | | | | | | | | | | 2 | 2 | 5 | 6 | 6 |
| | | | | | | | | | | 1 | 1 | 1 | 2.29 | 3 |

Part 6

| | | Pilocarpine 0.5% Plus Brimonidine 0.2% Eye Drops Vs. Placebo to Reduce The Need For Reading | | | | | | | | | | | | |
|---|---|---|---|---|---|---|---|---|---|---|---|---|---|---|
| | | Glasses (X 7 DAYS) | | | | | | | | | | | | |
| | | | | | Day 6 | | | | | Day 7 | | | | |
| | Pre-treatment | | | | Near | Near | Near | Near | Near | Near | Near | Near | Near | Near |
| | | | Near | — | Va(j) | Va(j) | Va(j) | Va(j) | Va(j) | Va(j) | Va(j) | Va(j) | Va(j) | Va(j) |
| | Age | Sex | VA(j) | — | 1 hr | 2 hr | 4 hr | 8 hr | 10 hr | 1 hr | 2 hr | 4 hr | 8 hr | 10 hr |
| Placebo Eye Drops | 49 | M | 6 | — | 6 | 6 | 6 | 6 | 6 | 6 | 6 | 6 | 6 | 6 |
| | 51 | M | 5 | — | 4 | 5 | 5 | 5 | 5 | 4 | 5 | 5 | 5 | 5 |
| | 50 | M | 5 | — | 4 | 5 | 5 | 5 | 5 | 4 | 5 | 5 | 5 | 5 |
| | 54 | F | 8 | — | 8 | 8 | 8 | 8 | 8 | 8 | 8 | 8 | 8 | 8 |
| | 49 | F | 5 | — | 4 | 5 | 5 | 5 | 5 | 4 | 5 | 5 | 5 | 5 |
| | 52 | M | 6 | — | 6 | 6 | 6 | 6 | 6 | 6 | 6 | 6 | 6 | 6 |
| | 47 | M | 5 | — | 5 | 5 | 5 | 5 | 5 | 5 | 5 | 5 | 5 | 5 |
| | 45 | F | 3 | — | 3 | 3 | 3 | 3 | 3 | 2 | 3 | 3 | 3 | 3 |
| | 54 | M | 8 | — | 8 | 8 | 8 | 8 | 8 | 8 | 8 | 8 | 8 | 8 |
| | 53 | F | 8 | — | 7 | 8 | 8 | 8 | 8 | 7 | 8 | 8 | 8 | 8 |
| | 49 | F | 6 | — | 6 | 6 | 6 | 6 | 6 | 6 | 6 | 6 | 6 | 6 |
| | 55 | F | 8 | — | 8 | 8 | 8 | 8 | 8 | 8 | 8 | 8 | 8 | 8 |
| | 52 | M | 8 | — | 8 | 8 | 8 | 8 | 8 | 8 | 8 | 8 | 8 | 8 |
| | 46 | F | 6 | — | 5 | 6 | 6 | 6 | 6 | 5 | 6 | 5 | 6 | 6 |
| | 48 | F | 6 | — | 5 | 6 | 6 | 6 | 6 | 6 | 6 | 6 | 6 | 6 |
| | 50 | M | 5 | — | 5 | 5 | 5 | 5 | 5 | 5 | 5 | 5 | 5 | 5 |
| | 50 | M | 6 | — | 6 | 6 | 6 | 6 | 6 | 6 | 6 | 6 | 6 | 6 |
| | 47 | F | 5 | — | 3 | 5 | 5 | 8 | 5 | 3 | 5 | 5 | 5 | 5 |
| | 54 | M | 10 | — | 10 | 10 | 10 | 10 | 10 | 10 | 10 | 10 | 10 | 10 |
| | 45 | F | 3 | — | 2 | 3 | 3 | 3 | 3 | 3 | 3 | 3 | 3 | 3 |
| | SUM | | 122 | — | 113 | 122 | 122 | 122 | 122 | 116 | 122 | 122 | 122 | 122 |
| | AVERAGE | | 6.1 | — | 5.65 | 6.1 | 6.1 | 6.1 | 6.1 | 5.8 | 6.1 | 6.1 | 6.1 | 6.1 |
| | | | | | | | | | | Averages | | | | |
| | | | | | | | | | | 1 hr = | 2 hr = | 4 hr = | 84 hr = | 10 hr = |
| | | | | | | | | | | Average | Average | Average | Average | Average |
| | | | | | | | | | | Near | Near | Near | Near | Near |
| | | | | | | | | | | VA(j) | VA(j) | VA(j) | VA(j) | VA(j) |
| | | | | | | | | | | 6 | 6 | 6 | 6 | 6 |
| | | | | | | | | | | 4.57 | 5 | 5 | 5 | 5 |
| | | | | | | | | | | 4 | 5 | 5 | 5 | 5 |
| | | | | | | | | | | 7.86 | 8 | 8 | 8 | 8.14 |
| | | | | | | | | | | 4 | 5 | 5 | 5 | 5 |
| | | | | | | | | | | 6 | 6 | 6 | 6 | 6 |
| | | | | | | | | | | 5 | 5 | 5 | 5 | 5 |
| | | | | | | | | | | 2.43 | 3 | 3 | 3 | 3 |
| | | | | | | | | | | 8 | 8 | 8 | 8 | 8 |
| | | | | | | | | | | 7.29 | 8 | 8 | 8 | 8 |
| | | | | | | | | | | 5.86 | 6 | 6 | 6 | 6 |
| | | | | | | | | | | 7071 | 8 | 8 | 8 | 8 |
| | | | | | | | | | | 8 | 8 | 8 | 8 | 8 |
| | | | | | | | | | | 5.29 | 5.86 | 6 | 6 | 6 |
| | | | | | | | | | | 5.71 | 6 | 6 | 6 | 6 |
| | | | | | | | | | | 5 | 5 | 5 | 5 | 5 |
| | | | | | | | | | | 6 | 6 | 6 | 6 | 6 |
| | | | | | | | | | | 3 | 4.71 | 5 | 5 | 5 |
| | | | | | | | | | | 10 | 10 | 10 | 10 | 10 |
| | | | | | | | | | | 2.14 | 3 | 3 | 3 | 3 |
| | | | | | | | | | | 5.69 | 6.08 | 6.1 | 6.1 | 6.11 |

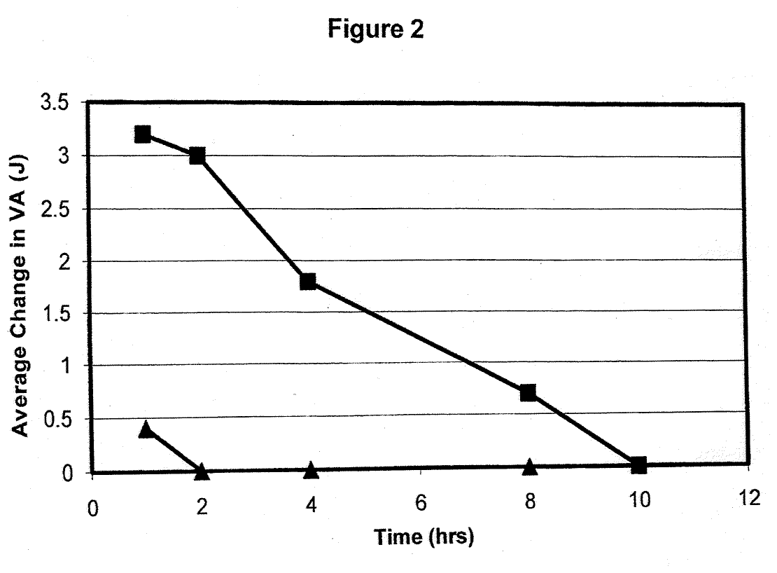

FIG. 2 show the average change in visual acuity at 1, 2, 4, 8, and 10 hours after administration for the active drug and placebo arms. The solid squares represent the average change in visual acuity for the active drug arm whereas the solid triangles represent the average change in visual acuity for the placebo arm. As can be seen from the data, there is a residual effect of the drug eight hours after administration for the active drug arm, allowing patients to read without corrective lenses for several hours.

### Example 5

One study examined the use of carbachol with an alpha agonist (brimonidine) to reduce the effect of presbyopia (Improved Presbyopic Vision With Miotics, Abdelkader, Eye & Contact Lens 2015;0: 1-5, herein incorporated by reference).

A prospective, double-masked, randomized, placebo-controlled clinical trial included forty-eight naturally emmetropic and presbyopic subjects between 43 years and 56 years old with an uncorrected distance visual acuity of at least 20/20 in both eyes without additional ocular pathology. Presbyopia was considered present if an uncorrected end-point print size ≥Jaeger (J) 5 improved by ≥1 optotype with the use of a lens ≥+1.00 D. Subjects were divided into 2 groups. The treatment group (n=30 eyes) received a single dose of 2.25% carbachol plus 0.2% brimonidine eye drops. The control group (n=18 eyes) received placebo drops. Drops were given to all subjects in a masked fashion, in their non-dominant eye. The minimum post-treatment follow-up was 3 months. The subjects' pupil size and both near and distance visual acuities were evaluated pre- and post-treatment at 1, 2, 4, 8 and 10 hours, by a masked examiner at the same room illumination.

Statistically significant improvement in near visual acuity was seen in all subjects who received carbachol plus brimonidine drops (P<0.0001). The subjects liked the treatment and would use this therapy if it was available. There was no evidence of tolerance or tachyphylaxis during the study.

The treatment group received ophthalmic drops which contained two drugs: 2.25% carbachol and 0.2% brimonidine (treatment group). Placebo eye drops were used in some subjects as a control. The pharmacological treatment of the treatment group had multiple purposes: to stimulate the parasympathetic innervation, increasing depth of focus, and the accommodation and its potentiation and prolongation by an alpha agonist. The aim of this study was to evaluate in a masked fashion the efficacy of using of a parasympathomimetic drug together with an alpha agonist to create optically beneficial miosis to temporarily improve vision in presbyopia by improving the depth of focus.

Patients with myopia, hyperopia and astigmatism higher than 0.25 diopter as well as those with corneal, lens and vitreous opacities, pupil irregularities, anisocoria, amblyopia, chronic general pathologies and medications that would interact unfavorably with carbachol and brimonidine were excluded. A single dose of carbachol (2.25% Isopto Carbachol, Alcon Inc., Fort Worth, Tex., USA) plus 0.2% brimonidine, or placebo, was administered in a masked fashion in the non-dominant eye of the subjects. Subjects were then instructed to use the eye drops once daily at home for 3 months.

The mean age of the treatment group was 50.83±4.57 years (range, 43-56 years); 16 males and 14 females. The mean age of the control group was 49.8±3.1 years (range, 45-55 years); 8 males and 10 females. In the treatment group, the number of subjects ≥50 years was 16 and those <50 years was 14. In the control group, the number of subjects ≥50 years was 9 and those <50 years was 9. No statistically significant difference in mean age or sex was found among the 2 groups. Table 3 summarizes the demographic data of the subjects of both groups.

TABLE 3
Demographic Data for treatment and control groups

| | Treatment group | Control group |
|---|---|---|
| Items | (n = 30 subjects) | (n = 18 subjects) |
| Male:Female | 16:14 | 8:10 |
| Age (mean [SD] yrs) | 50.83 [4.57] | 49.8 [3.1] |
| ≥50 years | 16 | 9 |
| <50 years | 14 | 9 |

On day 1, in the ≥50-year-old treatment group (2.25% carbachol plus 0.2% brimonidine), the mean near visual acuity (NVA) improved significantly from J-7.68±1.62 before treatment to J-3±1.26 at 1 hour (P<0.0001), J-3.4±1.4 at 2 hours (P<0.0001), J-4±1.26 at 4 hours (P<0.0001), J-4.75±1.09 at 8 hours (P<0.0001) and J-5.6±1.3 at 10 hours (P<0.0001) post-treatment.

In the <50-year-old treatment group (2.25% carbachol plus 0.2% brimonidine), the mean near visual acuity (NVA) improved significantly from J-6.29±0.91 before treatment to J-2.5±0.94 at 1 hour (P<0.0001), J-3.14±0.86 at 2 hours (P=0.0001), J-3.71±0.91 at 4 hours (P<0.0001), J-4.64±0.74 at 8 hours (P<0.0001) and J-5.29±0.73 at 10 hours (P=0.0036) post-treatment.

No statistically significant difference in mean NVA and pupil size was found between both age groups before treatment and at any time point after treatment (P>0.05).

No statistically significant difference in mean NVA was found in the placebo

TABLE 4

| | | | Carbachol plus Brimonidine | | Placebo | |
|---|---|---|---|---|---|---|
| Time (hrs) | | | ≥50 yrs. | <50 yrs. | ≥50 yrs. | <50 yrs. |
| Pre- | | NVA | 7.68 | 6.29 | 6.77 | 5.22 |
| treatment | | | | | | |
| 1 | hour | NVA | 3 | 2.5 | 6.44 | 4.67 |
| 2 | hour | NVA | 3.4 | 3.14 | 6.77 | 5.22 |
| 4 | hour | NVA | 4 | 3.71 | 6.77 | 5.22 |
| 8 | hour | NVA | 4.75 | 4.64 | 6.77 | 5.22 |
| 10 | hour | NVA | 5.6 | 5.29 | 6.77 | 5.22 |

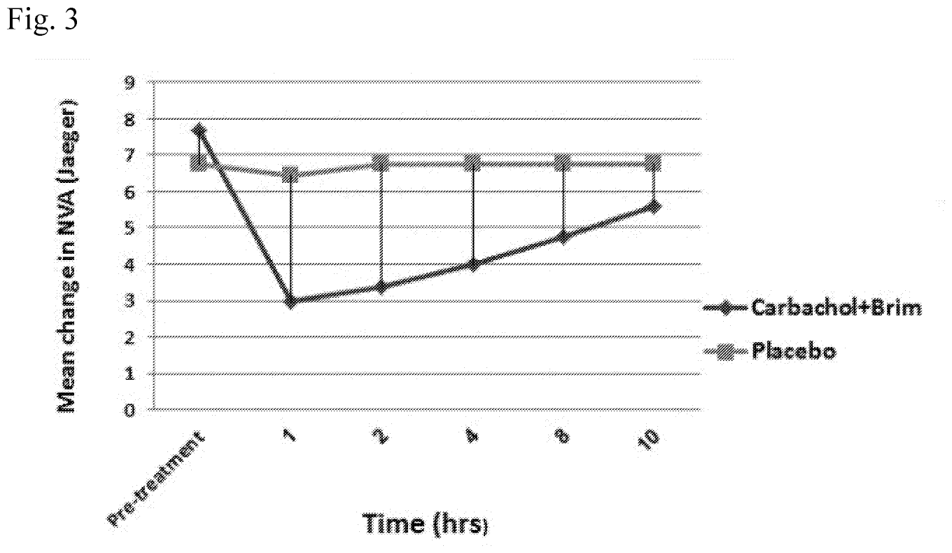

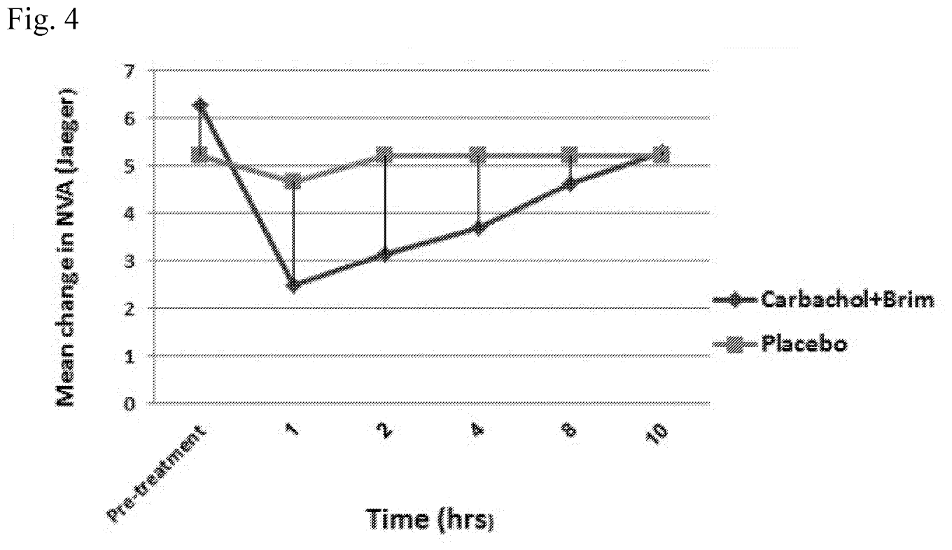

(control) group before treatment and at any time point after treatment. Data is summarized in Table 4. Table 4 shows the mean change in near visual acuity (NVA) (Jaeger) over time for treatment (carbachol plus brimonidine) and control (placebo) groups. FIGS. 3 and 4 show the mean change in near visual acuity (Jaeger) over time for treatment and control groups.

No statistically significant difference in mean NVA was found at 2 hours after administering the drops between day 1 (J-3.4±1.4) and day 7 (J-3±0.73) (P=0.29) for the ≥50-year-old treatment group after 1 week.

No statistically significant difference in mean NVA was found at 2 hours after administering the drops between day 1 (J-3.14±0.86) and at one week (J-2.64±0.74) (P=0.11). for the <50-year-old treatment group after 1 week.

No statistically significant difference in mean NVA was found at 4 hours after administering the drops between day 1 (J-4±1.26) and at one month (J-3.56±0.73) (P=0.23) for the ≥50-year-old treatment group.

No statistically significant difference in mean NVA was found at 4 hours after administering the drops between day 1 (J-3.71±0.91) and at one month (J-3.29±0.61) (P=0.15) for the <50-year-old group.

No statistically significant difference in mean NVA was found at 8 hours of installing the drops between day 1 (J-4.75±1.09) and at 2 month (J-4.13±0.81) (P=0.07) for the ≥50-year-old treatment group.

No statistically significant difference in mean NVA was found at 8 hours after administering the drops between day 1 (J-4.64±0.74) and at 2 month (J-4.21±0.43) (P=0.07) for the <50-year-old treatment group.

No statistically significant difference in mean NVA was found at 10 hours after administering the drops between day 1 (J-5.6±1.3) and at 3 month (J-5.13±0.81) (P=0.2) for the ≥50-year-old treatment group.

No statistically significant difference in mean (NVA) was found at 10 hours after administering the drops between day 1 (J-5.29±0.73) and at 3 month (J-4.93±0.62) (P=0.17) for the <50-year-old treatment group.

The uncorrected distance visual acuity was 20/20 of both eyes in all subjects before treatment and remained at 20/20 at all time periods after treatment.

All presbyopes in this study who received carbachol plus brimonidine liked and would use this therapy if it was available. They all stopped using glasses for near vision and were satisfied with both their near and distance vision. Twelve subjects out of 30 (40%) reported that the effect was excellent for the first 8 hours then gradually faded. The improvement in near vision was satisfactory for those subjects during their working day.

None of the participants in the placebo group would use the placebo. All subjects who received placebo reported that the drops did not improve their near vision, and they discontinued using the drops.

No serious adverse ocular effects were observed during the study period for the carbachol plus brimonidine treatment group. No conjunctival hyperemia or red eye was observed. A mild burning sensation was noted in one subject (3.3%). A dull headache was reported in 10% of all subjects. The drops showed excellent safety and stability. Temporary dimness for the first couple of weeks was reported by one subject (3.3%). However, this subject reported that this symptom was mild and temporary and did not induce him to discontinue the drops. Systemic side effects as bradychardia, bronchospasm and digestive problems were not found. There was no evidence of tolerance or tachyphylaxis and the effect of the drops persisted during the entire follow-up period.

For the placebo group, a mild burning sensation was reported in 2 subjects (11.1% J).

This example used 2.25% carbachol and an alpha agonist (0.2% brimonidine) to improve vision in presbyopia through increased depth of focus in participants in both their forties and fifties. Increased depth of focus allowed many presbyopes to benefit from using the drops. Both drugs are FDA approved and have been used for years as safe and effective for glaucoma. Placebo drops were used as a control. It is believed that the technique creates a pinhole effect pharmacologically increasing the depth of focus from a smaller pupil. In monocular treatment, the vision in the fellow eye with the normal pupil will have some blurry near vision, but distant objects are clear and there is no diminished light perception. When the images are merged, all subjects of the treatment group (except for one) had clear focus at near and distance with no perception of dimness except in one subject (3.3%). Treating one eye only does not cause symptoms of dimness as the brain fills in brightness from the other eye. Carbachol and brimonidine can be used once daily to achieve a 10-hour effect. Brimonidine has little effect on the photopic pupil, but has been effectively used for many years to prevent excessive pupil dilatation in the dark, and thereby reduces scotopic symptoms, usually from the peripheral cornea after refractive surgery. It has not been used to ameliorate presbyopia.

The synergistic effect between carbachol and brimonidine allows one application of drops to produce miosis sufficient to improve near vision enough for most people all day long. The combination between carbachol and brimonidine was active and their effect lasted longer. Distance vision was preserved in all subjects, so that no monovision symptoms were reported. The treatment of only one eye minimized symptoms of dimness; synergism permitted use of lower doses of miotics and reduced symptoms of headache, and brimonidine eliminated any tendency of the parasympathomimetic to cause hyperemia. In this study, there was no evidence of tolerance or tachyphylaxis and the effect of the drops (carbachol plus brimonidine) persisted during the study period. No ocular complications were detected in any treated eyes during the entire follow-up period. Although near visual acuity was significantly improved, it did not return to J 1 in most of the subjects. NVA returned to J 1 in 4 subjects (13.3%) of the study group, 2 subjects for each age group.

The treatment of presbyopia with one drop a day of carbachol and brimonidine in the non-dominant eye permitted acceptable reading vision for many presbyopes even in older subjects. Because of increased depth of focus from the smaller pupil, it did not blur distance vision or intermediate vision, as does typical monovision therapy, and the perception of normal brightness in the untreated eye eliminated symptoms of dimming from the smaller pupil of the treated eye. This active combination would also improve low non-presbyopic hyperopes and can be used with glasses if necessary.

### Example 6

Another study evaluated the efficacy of using carbachol and brimonidine to improve vision in presbyopia, myopia and hyperopia.

The medical treatment in this study was designed to improve vision in patients with refractive errors using ophthalmic drops, which contained two drugs: a parasympathomimetic (carbachol) of various concentrations and an alpha agonist agent (0.2% brimonidine). 0.2% brimonidine alone and placebo eye drops were used in some subjects as a control. The pharmacological treatment of the treatment group stimulated the parasympathetic innervation primarily by improving depth of focus and perhaps the accommodation and its potentiation and prolongation by an alpha agonist. The study evaluated in a blind study the efficacy of using of a parasympathomimetic drug of different concentrations together with an alpha agonist to create optically beneficial miosis to temporarily treat different types of presbyopia (emmetropic, myopic and hyperopic).

One hundred and seventy seven presbyopic subjects with a mean age of 49.8±3.9 years (range 41-57 years); 96 males and 81 females were recruited in this study. The participants in the study all gave written informed consent. The pharmacological stimulation protocol was developed in accordance with the methods disclosed in U.S. Pat. No. 8,299,079, herein incorporated by reference. All subjects were in good physical and ocular health and completed a questionnaire to ascertain any contraindications for participation or predisposition to complications (e.g. heart or respiratory conditions, migraines, high myopia, ocular or systemic medications, or ocular surgeries). All subjects had a fully dilated eye examination before they were considered eligible for the study. The examination screened for contraindications to the drugs, susceptibility to retinal detachment, ocular pathology, or peripheral retinal degeneration. Exclusion criteria concerned patients with myopia or astigmatism higher than 0.75 diopter, and hypermetropia greater than 2 diopters in either eye as well as those with corneal, lens and vitreous opacities, pupil irregularities, anisocoria, amblyopia, chronic general pathologies and medications that would interact unfavorably with carbachol and brimonidine. Subjects were screened for known sensitivities to the drugs or conditions that would preclude the use of these drops. During the study, the subjects were closely monitored and regularly asked to report on any ocular, systemic, or physiological reactions they experienced. Atropine was available in the event of adverse effects, although none was reported. Different groups of presbyopic subjects were differentiated. Group 1 included 66 emmetropic presbyopes (n=66 eyes), Group 2 included 55 myopic presbyopes (≤−0.75 D sphere, n=55 eyes) and group 3 included 56 hyperopic presbyopes (≤+2D sphere, n=112 eyes). Each group was then subdivided according to the age into fifty years or more and below fifty years.

A single dose of different concentrations of carbachol (Isopto Carbachol 2.25%, 1.5%, 3%, Alcon Inc., Fort Worth, Tex., USA) plus 0.2% brimonidine or 0.2% brimonidine alone or placebo was instilled in a masked fashion in the non-dominant eyes of Groups 1 and, 2 and in both eyes of Group 3, respectively. Initial pupil size and both near and distance visual acuities were documented before treatment and at 1, 2, 4, 8 and 10 hours after treatment at the same room illumination. Subjects were monitored and subjected to complete ocular examination including visual acuity evaluation and slit-lamp biomicroscopy after one week of treatment and monthly during the first three months to evaluate dosage, satisfaction, adverse effects and complications (for instance, retinal detachment, pigment dispersion, posterior synechiae and intraocular inflammation). Subjects were instructed to use the eye drops once daily during the follow up period. Distance visual acuity was measured using the standard Snellen projector chart. The Jaeger Eye Chart was used to measure near visual acuity. Any adverse symptoms and subject satisfaction with near and distance vision were also monitored.

Statistical analysis was performed using the Student's t-test and p value of less than 0.05 was considered statistically significant. Data were expressed as mean, range, and standard deviation (SD).

The mean subject age (years) was 50.3±4 (range 43-57) in the emmetropic presbyopes (Group 1), 50.8±3.7 (range 45-57) in the myopic presbyopes (Group 2), and 48.3±3.8 (range 41-56) in the hyperopic presbyopes (Group 3). In group 1, the number of subjects ≥50 years was 34 and those <50 years was 32. In group 2, the number of subjects ≥50 years was 28 and those <50 years was 27. In group 3, the number of subjects ≥50 years was 29 and those <50 years was 27. No statistically significant difference in mean age or sex was found among the 3 groups.

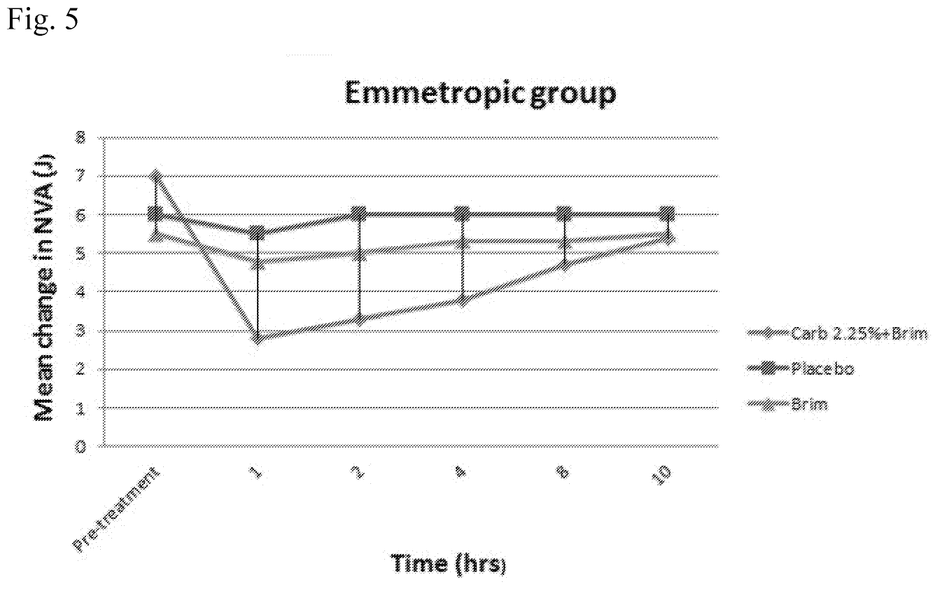

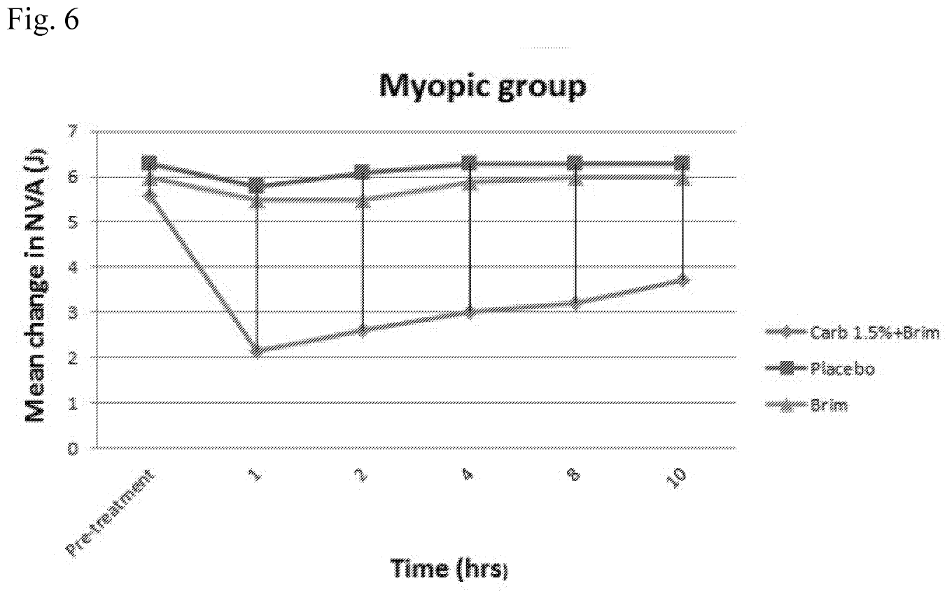

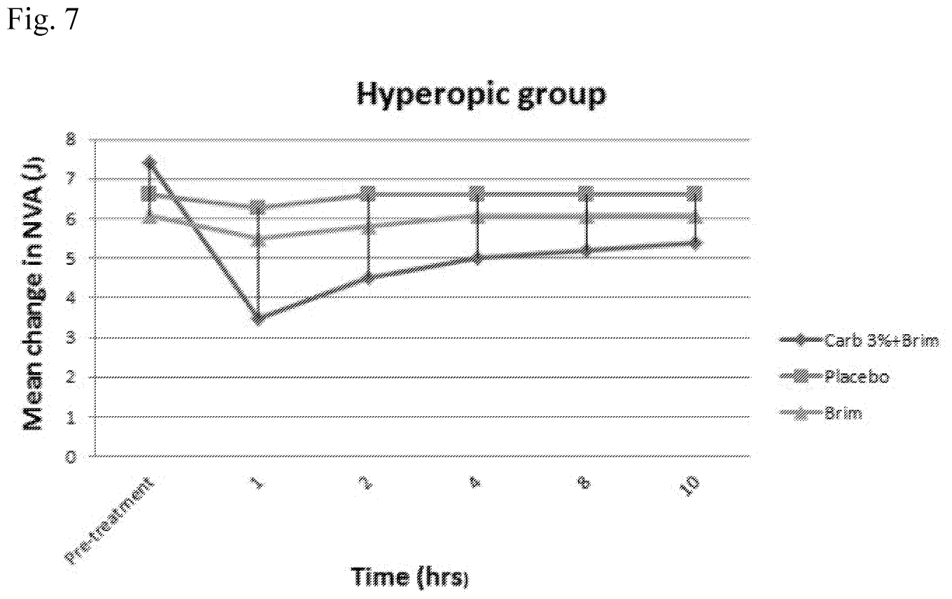

FIGS. 5, 6, and 7 show the mean change in near visual acuity (J) over time for emmetropic, myopic and hyperopic presbyopic groups.

Group 1 (Emmetropic presbyopes):

The concentration of carbachol used in this group was 2.25%. The mean pre-treatment manifest refraction was −0.1±0.12 D. The mean post-treatment refraction at 1, 2, 4, 8, 10 hours was −0.6±0.14 D, −0.5±0.12 D, −0.48±0.09 D, −0.4±0.1 D, and −0.38±0.12 D, respectively.

As shown in Table 5, in the ≥50 years old treatment group (carbachol 2.25% plus brimonidine 0.2%), the mean near visual acuity (NVA) improved significantly from J-7.6±1.62 before treatment to J-3±1.26 at 1 hour (P<0.0001), J-3.4±1.4 at 2 hours (P<0.0001), J-4±1.26 at 4 hours (P<0.0001), J-4.75±1.09 at 8 hours (P<0.0001) and J-5.6±1.3 at 10 hours (P=0.00004) post-treatment.

TABLE 5
Group 1 (Pure presbyopes) ≥ 50 years
2.25% Carbachol plus brimonidine vs placebo vs brimonidine

| | | | | Pre- | | | | | |
|---|---|---|---|---|---|---|---|---|---|
| | | | | treatment | Post-Treatment | | | | |
| | | | | Near | NVa | NVa | Nva | Nva | Nva |
| | Patients′ | Age | | VA | 1 h | 2 h | 4 h | 8 h | 10 h |
| | number | (yrs) | sex | (J) | (J) | (J) | (J) | (J) | (J) |
| Carbachol 2.25% | 33 | 55 | f | 5 | 2 | 2 | 3 | 4 | 4 |
| plus brimonidine | 34 | 55 | f | 8 | 3 | 3 | 4 | 5 | 5 |
| 0.2% | 35 | 55 | f | 10 | 5 | 6 | 6 | 6 | 7 |
| | 36 | 54 | m | 7 | 4 | 4 | 4 | 5 | 6 |
| | 37 | 54 | m | 7 | 2 | 3 | 5 | 4 | 4 |
| | 38 | 55 | m | 8 | 1 | 1 | 2 | 3 | 8 |
| | 39 | 56 | m | 10 | 4 | 5 | 5 | 6 | 5 |
| | 40 | 55 | m | 7 | 3 | 3 | 3 | 5 | 7 |
| | 41 | 55 | m | 7 | 2 | 2 | 3 | 4 | 4 |
| | 42 | 55 | f | 7 | 3 | 3 | 4 | 5 | 5 |
| | 43 | 55 | m | 10 | 5 | 6 | 6 | 6 | 7 |
| | 44 | 54 | m | 7 | 4 | 5 | 4 | 5 | 6 |
| | 45 | 55 | f | 5 | 2 | 3 | 5 | 4 | 4 |
| | 46 | 55 | f | 7 | 1 | 3 | 2 | 3 | 7 |
| | 47 | 56 | m | 10 | 4 | 4 | 5 | 6 | 5 |
| | 48 | 53 | m | 8 | 3 | 2 | 3 | 5 | 6 |
| | [symbol] | [symbol] | | [symbol] | [symbol] | [symbol] | [symbol] | [symbol] | [symbol] |
| Placebo Eye Drops | 49 | 51 | M | 5 | 5 | 5 | 5 | 5 | 5 |
| | 50 | 50 | M | 5 | 4 | 5 | 5 | 5 | 5 |
| | 51 | 54 | F | 8 | 8 | 8 | 8 | 8 | 8 |
| | 52 | 52 | M | 6 | 6 | 6 | 6 | 6 | 6 |
| | 53 | 54 | M | 8 | 8 | 8 | 8 | 8 | 8 |
| | 54 | 53 | F | 8 | 8 | 8 | 8 | 8 | 8 |
| | 55 | 55 | F | 8 | 6 | 8 | 8 | 8 | 8 |
| | 56 | 52 | M | 8 | 8 | 8 | 8 | 8 | 8 |
| | 57 | 50 | M | 5 | 5 | 5 | 5 | 5 | 5 |
| | [symbol] | [symbol] | | [symbol] | [symbol] | [symbol] | [symbol] | [symbol] | [symbol] |
| Brimonidine 0.2% | 58 | 55 | F | 8 | 8 | 8 | 8 | 8 | 8 |
| | 59 | 52 | M | 6 | 5 | 5 | 6 | 6 | 6 |
| | 60 | 50 | F | 5 | 4 | 5 | 5 | 5 | 5 |
| | 61 | 53 | F | 6 | 5 | 5 | 5 | 5 | 6 |
| | 62 | 57 | M | 8 | 7 | 7 | 8 | 8 | 8 |
| | 63 | 55 | F | 6 | 5 | 6 | 6 | 6 | 6 |
| | 64 | 52 | M | 6 | 5 | 5 | 6 | 6 | 6 |
| | 65 | 50 | M | 4 | 3 | 4 | 4 | 4 | 4 |
| | 66 | 54 | F | 6 | 6 | 6 | 6 | 6 | 6 |
| | [symbol] | [symbol] | | [symbol] | [symbol] | [symbol] | [symbol] | [symbol] | [symbol] |

As shown in Table 6, in the <50 years old treatment group (carbachol 2.25% plus brimonidine 0.2%), the mean near visual acuity (NVA) improved significantly from J-6.29±0.91 before treatment to J-2.5±0.94 at 1 hour (P<0.0001), J-3.14±0.86 at 2 hours (P=0.0001), J-3.71±0.91 at 4 hours (P<0.0001), J-4.64±0.74 at 8 hours (P<0.0001) and J-5.29±0.73 at 10 hours (P=0.0036) post-treatment.

TABLE 6
Group 1 (Pure presbyopes) <50 years
2.25% Carbachol plus brimonidine 0.2% Eye Drops vs Placebo vs brimonidine

| | | | | Pre- | | | | | |
|---|---|---|---|---|---|---|---|---|---|
| | | | | Treatment | Post-Treatment | | | | |
| | | | | Near | NVa | NVa | Nva | Nva | Nva |
| | Patients′ | age | | VA | 1 h | 2 h | 4 h | 8 h | 10 h |
| | number | (yrs) | sex | (J) | (J) | (J) | (J) | (J) | (J) |
| Carbachol 2.25% | 1 | 48 | f | 6 | 1 | 2 | 2 | 4 | 4 |
| plus brimonidine | 2 | 47 | m | 8 | 3 | 3 | 4 | 5 | 6 |
| 0.2% | 3 | 47 | m | 6 | 2 | 4 | 4 | 4 | 5 |
| | 4 | 44 | m | 6 | 3 | 3 | 4 | 5 | 5 |
| | 5 | 49 | m | 5 | 2 | 2 | 3 | 6 | 6 |
| | 6 | 46 | m | 8 | 4 | 4 | 4 | 4 | 5 |
| | 7 | 47 | f | 7 | 3 | 4 | 5 | 5 | 6 |
| | 8 | 49 | f | 6 | 3 | 3 | 4 | 4 | 5 |
| | 9 | 45 | m | 6 | 4 | 3 | 4 | 6 | 5 |
| | 10 | 43 | f | 5 | 2 | 2 | 3 | 4 | 4 |
| | 11 | 44 | m | 6 | 2 | 4 | 4 | 4 | 6 |
| | 12 | 49 | m | 7 | 3 | 4 | 2 | 5 | 5 |
| | 13 | 44 | m | 6 | 3 | 4 | 4 | 4 | 6 |
| | 14 | 46 | m | 6 | 1 | 2 | 5 | 5 | 6 |
| | [symbol] | [symbol] | | [symbol] | [symbol] | [symbol] | [symbol] | [symbol] | [symbol] |
| Placebo Eye Drops | 15 | 49 | M | 6 | 6 | 6 | 6 | 6 | 6 |
| | 16 | 49 | F | 5 | 4 | 5 | 5 | 5 | 5 |
| | 17 | 47 | M | 5 | 5 | 5 | 5 | 5 | 5 |
| | 18 | 45 | F | 4 | 4 | 4 | 4 | 4 | 4 |
| | 19 | 49 | F | 6 | 5 | 6 | 6 | 6 | 6 |
| | 20 | 46 | F | 6 | 5 | 6 | 6 | 6 | 6 |
| | 21 | 48 | F | 6 | 6 | 6 | 6 | 6 | 6 |
| | 22 | 47 | F | 5 | 4 | 5 | 5 | 5 | 5 |
| | 23 | 45 | F | 4 | 3 | 4 | 4 | 4 | 4 |
| | [symbol] | [symbol] | | [symbol] | [symbol] | [symbol] | [symbol] | [symbol] | [symbol] |
| Brimonidine 0.2% | 24 | 45 | F | 4 | 3 | 4 | 4 | 4 | 4 |
| | 25 | 43 | M | 3 | 3 | 3 | 3 | 3 | 3 |
| | 26 | 48 | M | 5 | 4 | 4 | 4 | 4 | 5 |
| | 27 | 46 | M | 6 | 5 | 5 | 5 | 5 | 6 |
| | 28 | 47 | M | 5 | 4 | 4 | 5 | 5 | 5 |
| | 29 | 49 | F | 6 | 5 | 6 | 6 | 6 | 6 |
| | 30 | 48 | F | 6 | 5 | 5 | 6 | 6 | 6 |
| | 31 | 45 | M | 4 | 4 | 4 | 4 | 4 | 4 |
| | 32 | 47 | F | 6 | 5 | 5 | 5 | 5 | 6 |
| | [symbol] | [symbol] | | [symbol] | [symbol] | [symbol] | [symbol] | [symbol] | [symbol] |

No statistically significant difference in mean NVA was found between ≥50 and <50 groups before treatment (P = 0.5) and at 1 hour (P = 0.49), 2 hours (P = 0.7), 4 hours (P = 0.64), 8 hours (P = 0.94) and 10 hours (P = 0.57) post-treatment.

No statistically significant difference in mean NVA was found in the placebo or 0.2% brimonidine alone groups before treatment and at any time point after treatment.

Group 2 (Myopic Presbyopes):

The concentration of carbachol used in this group was 1.5%. The mean pre-treatment spherical refractive error was −0.63±0.13 dioptres and mean refractive astigmatism amounted to 0.17±0.24 dioptres. The mean post-treatment spherical refraction at 1, 2, 4, 8, 10 hours was −0.8±0.18 D, −0.71±0.22 D, −0.69±0.21 D, −0.67±0.23 D, −0.65±0.18 D, respectively.

As shown in Table 7, in the ≥50 years old treatment group (carbachol 1.5% plus brimonidine 0.2%), the mean near visual acuity NVA improved significantly from J-5.5±1.37 before treatment to J-2.25±0.58 at 1 hour (P<0.0001), J-2.75±0.58 at 2 hours (P <0.0001), J-3.13±0.72 at 4 hours (P<0.0001), J-3.25±0.68 at 8 hours (P<0.0001) and J-3.63±0.89 at 10 hours (P<0.0001) post-treatment.

TABLE 7
Group 2 (Myopic presbyopes) ≥50 years
1.5% Carbachol plus brimonidine vs placebo vs brimonidine

| | | | | Pre- | | | | | |
|---|---|---|---|---|---|---|---|---|---|
| | | | | Treatment | Post-Treatment | | | | |
| | | | | Near | NVa | NVa | Nva | Nva | Nva |
| | Patients′ | Age | | VA | 1 h | 2 h | 4 h | 8 h | 10 h |
| | number | (yrs) | sex | (J) | (J) | (J) | (J) | (J) | (J) |
| Carbachol 1.5% & | 28 | 52 | f | 5 | 2 | 2 | 3 | 3 | 3 |
| brimonidine 0.2% | 29 | 55 | m | 5 | 1 | 3 | 4 | 4 | 4 |
| | 30 | 55 | f | 4 | 2 | 3 | 2 | 2 | 3 |
| | 31 | 54 | m | 7 | 2 | 2 | 4 | 3 | 3 |
| | 32 | 53 | f | 4 | 3 | 3 | 4 | 4 | 4 |
| | 33 | 55 | m | 8 | 3 | 3 | 3 | 4 | 5 |
| | 34 | 55 | f | 7 | 3 | 3 | 3 | 3 | 4 |
| | 35 | 54 | f | 4 | 2 | 2 | 2 | 3 | 3 |
| | 36 | 52 | m | 5 | 2 | 2 | 3 | 3 | 3 |
| | 37 | 55 | f | 5 | 2 | 3 | 4 | 4 | 4 |
| | 38 | 55 | m | 5 | 2 | 3 | 2 | 2 | 3 |
| | 39 | 55 | m | 6 | 2 | 3 | 3 | 3 | 3 |
| | 40 | 54 | m | 4 | 2 | 4 | 4 | 4 | 4 |
| | 41 | 52 | m | 8 | 3 | 3 | 3 | 4 | 6 |
| | 42 | 55 | f | 6 | 3 | 3 | 3 | 3 | 3 |
| | 43 | 54 | f | 5 | 2 | 2 | 3 | 3 | 3 |
| | [symbol] | [symbol] | | [symbol] | [symbol] | [symbol] | [symbol] | [symbol] | [symbol] |
| Placebo Drops | 44 | 55 | F | 8 | 7 | 7 | 8 | 8 | 8 |
| | 45 | 53 | F | 6 | 6 | 6 | 6 | 6 | 6 |
| | 46 | 55 | M | 6 | 6 | 6 | 6 | 6 | 6 |
| | 47 | 56 | M | 6 | 5 | 6 | 6 | 6 | 6 |
| | 48 | 55 | M | 7 | 6 | 7 | 7 | 7 | 7 |
| | 49 | 54 | F | 6 | 6 | 6 | 6 | 6 | 6 |
| | [symbol] | [symbol] | | [symbol] | [symbol] | [symbol] | [symbol] | [symbol] | [symbol] |
| Brimonidine 0.2% | 50 | 51 | F | 5 | 4 | 5 | 5 | 5 | 5 |
| | 51 | 55 | F | 6 | 6 | 6 | 6 | 6 | 6 |
| | 52 | 57 | M | 6 | 6 | 6 | 6 | 6 | 6 |
| | 53 | 54 | F | 6 | 5 | 5 | 5 | 6 | 6 |
| | 54 | 55 | F | 7 | 6 | 6 | 7 | 7 | 7 |
| | 55 | 53 | M | 6 | 6 | 6 | 6 | 6 | 6 |
| | [symbol] | [symbol] | | [symbol] | [symbol] | [symbol] | [symbol] | [symbol] | [symbol] |

As shown in Table 8, in the <50 years old treatment group (carbachol 1.5% plus brimonidine 0.2%), the mean near visual acuity (NVA) improved significantly from J-5.86±0.7 before treatment to J-2±0.55 at 1 hour (P<0.0001), J-2.57±1.1 at 2 hours (P=0.0001), J-2.86±0.86 at 4 hours (P<0.0001), J-3.29±0.9 at 8 hours (P<0.0001) and J-3.86±0.86 at 10 hours (P=0.0002) post-treatment.

TABLE 8
Group 2 (Myopic presbyopes) <50 years
1.5% Carbachol plus brimonidine vs placebo vs brimonidine

| | | | | Pre- | | | | | |
|---|---|---|---|---|---|---|---|---|---|
| | | | | Treatment | Post-Treatment | | | | |
| | | | | Near | NVa | NVa | Nva | Nva | Nva |
| | Patients′ | Age | | VA | 1 h | 2 h | 4 h | 8 h | 10 h |
| | number | (yrs) | sex | (J) | (J) | (J) | (J) | (J) | (J) |
| Carbachol 1.5% & | 1 | 47 | F | 6 | 2 | 3 | 3 | 4 | 4 |
| brimonidine 0.2% | 2 | 48 | m | 7 | 1 | 1 | 2 | 2 | 3 |
| | 3 | 49 | m | 5 | 2 | 1 | 1 | 2 | 3 |
| | 4 | 48 | m | 6 | 2 | 3 | 4 | 4 | 4 |
| | 5 | 47 | m | 5 | 3 | 4 | 4 | 4 | 5 |
| | 6 | 47 | m | 6 | 2 | 3 | 3 | 3 | 4 |
| | 7 | 45 | m | 6 | 2 | 3 | 3 | 3 | 3 |
| | 8 | 46 | m | 6 | 2 | 3 | 3 | 4 | 4 |
| | 9 | 46 | m | 7 | 1 | 1 | 2 | 3 | 4 |
| | 10 | 47 | F | 5 | 2 | 1 | 2 | 2 | 3 |
| | 11 | 48 | F | 7 | 3 | 3 | 4 | 4 | 4 |
| | 12 | 46 | F | 6 | 2 | 3 | 3 | 5 | 6 |
| | 13 | 46 | m | 5 | 2 | 4 | 3 | 3 | 4 |
| | 14 | 47 | F | 5 | 2 | 3 | 3 | 3 | 3 |
| | [symbol] | [symbol] | | [symbol] | [symbol] | [symbol] | [symbol] | [symbol] | [symbol] |
| Placebo Drops | 15 | 49 | F | 6 | 5 | 6 | 6 | 6 | 6 |
| | 16 | 47 | F | 5 | 5 | 5 | 5 | 5 | 5 |
| | 17 | 48 | M | 7 | 7 | 7 | 7 | 7 | 7 |
| | 18 | 49 | F | 7 | 6 | 6 | 7 | 7 | 7 |
| | 19 | 47 | F | 6 | 6 | 6 | 6 | 6 | 6 |
| | 20 | 48 | M | 6 | 5 | 6 | 6 | 6 | 6 |
| | [symbol] | [symbol] | | [symbol] | [symbol] | [symbol] | [symbol] | [symbol] | [symbol] |
| Brimonidine 0.2% | 21 | 48 | F | 7 | 6 | 6 | 7 | 7 | 7 |
| | 22 | 48 | M | 6 | 5 | 5 | 6 | 6 | 6 |
| | 23 | 46 | M | 6 | 6 | 6 | 6 | 6 | 6 |
| | 24 | 47 | M | 6 | 5 | 5 | 6 | 6 | 6 |
| | 25 | 45 | F | 5 | 5 | 5 | 5 | 5 | 5 |
| | 26 | 49 | M | 6 | 6 | 6 | 6 | 6 | 6 |
| | 27 | 48 | F | 6 | 6 | 5 | 6 | 6 | 6 |
| | [symbol] | [symbol] | | [symbol] | [symbol] | [symbol] | [symbol] | [symbol] | [symbol] |

No statistically significant difference in mean NVA was found between ≥50 and <50 groups before treatment (P=0.6) and at 1 hour (P=0.6), 2 hours (P=0.7), 4 hours (P=0.59), 8 hours (P=0.9) and 10 hours (P=0.54) post-treatment.

No statistically significant difference in mean NVA was found in the placebo or 0.2% brimonidine alone groups before treatment and at any time point after treatment.

Group 3 (Hyperopic Presbyopes):

The concentration of carbachol used in this group was 3%. The mean pre-treatment spherical refractive error in both eyes was +1.16±0.43 dioptres and mean refractive astigmatism was 0.2±0.25 dioptres. The mean post-treatment spherical refraction in both eyes at 1, 2, 4, 8, 10 hours was +0.21±0.16 D, +0.24±0.17 D, +0.33±0.14 D, +0.41±0.15 D, +0.43±0.16 D, respectively.

As shown in Table 9, in the ≥50 years old treatment group (carbachol 3% plus brimonidine 0.2%), the mean near visual acuity (NVA) in both eyes improved significantly from J-7.5±1.86 before treatment to J-4±1.26 at 1 hour (P<0.0001), J-4.75±1.18 at 2 hours (P<0.0001), J-5.38±1.09 at 4 hours (P=0.0004), J-5.5±0.89 at 8 hours (P=0.0005) and J-5.69±0.79 at 10 hours (P=0.0012) post-treatment.

TABLE 9
Group 3 (Hyperopic presb) ≥50 years 3% Carbachol plus brimonidine vs
Placebo vs Brim

| | | | | Pre- | | | | | |
|---|---|---|---|---|---|---|---|---|---|
| | | | | Treatment | Post-Treatment | | | | |
| | | | | Near | NVa | NVa | Nva | Nva | Nva |
| | Patients′ | Age | | VA | 1 h | 2 h | 4 h | 8 h | 10 h |
| | number | (yrs) | sex | (J) | (J) | (J) | (J) | (J) | (J) |
| Carbachol 3% plus | 28 | 50 | m | 9 | 5 | 5 | 6 | 6 | 6 |
| brimonidine 0.2% | 29 | 50 | f | 10 | 6 | 7 | 7 | 7 | 7 |
| | 30 | 50 | m | 8 | 4 | 5 | 6 | 6 | 6 |
| | 31 | 51 | m | 6 | 4 | 4 | 4 | 4 | 5 |
| | 32 | 50 | f | 4 | 2 | 3 | 3 | 4 | 5 |
| | 33 | 52 | f | 9 | 3 | 4 | 6 | 6 | 5 |
| | 34 | 50 | m | 6 | 4 | 5 | 5 | 5 | 5 |
| | 35 | 50 | m | 8 | 3 | 4 | 5 | 5 | 6 |
| | 36 | 51 | f | 8 | 6 | 6 | 6 | 6 | 7 |
| | 37 | 51 | m | 10 | 6 | 7 | 7 | 7 | 7 |
| | 38 | 54 | f | 8 | 4 | 5 | 6 | 6 | 6 |
| | 39 | 50 | m | 7 | 4 | 4 | 4 | 5 | 5 |
| | 40 | 51 | f | 4 | 2 | 3 | 5 | 5 | 5 |
| | 41 | 50 | m | 9 | 4 | 4 | 6 | 6 | 6 |
| | 42 | 51 | m | 6 | 4 | 5 | 5 | 5 | 5 |
| | 43 | 52 | f | 8 | 3 | 5 | 5 | 5 | 5 |
| | [symbol] | [symbol] | | [symbol] | [symbol] | [symbol] | [symbol] | [symbol] | [symbol] |
| Placebo Drops | 44 | 51 | F | 6 | 5 | 6 | 6 | 6 | 6 |
| | 45 | 54 | F | 6 | 6 | 6 | 6 | 6 | 6 |
| | 46 | 53 | M | 8 | 8 | 8 | 8 | 8 | 8 |
| | 47 | 51 | M | 8 | 7 | 8 | 8 | 8 | 8 |
| | 48 | 55 | F | 8 | 8 | 8 | 8 | 8 | 8 |
| | 49 | 53 | M | 5 | 5 | 5 | 5 | 5 | 5 |
| | [symbol] | [symbol] | | [symbol] | [symbol] | [symbol] | [symbol] | [symbol] | [symbol] |
| Brimonidine 0.2% | 50 | 50 | F | 6 | 5 | 6 | 6 | 6 | 6 |
| | 51 | 51 | M | 6 | 6 | 6 | 6 | 6 | 6 |
| | 52 | 53 | M | 6 | 5 | 5 | 6 | 6 | 6 |
| | 53 | 56 | M | 8 | 7 | 7 | 8 | 8 | 8 |
| | 54 | 54 | F | 7 | 6 | 6 | 7 | 7 | 7 |
| | 55 | 52 | M | 5 | 5 | 5 | 5 | 5 | 5 |
| | 56 | 51 | F | 5 | 5 | 5 | 5 | 5 | 5 |
| | [symbol] | [symbol] | | [symbol] | [symbol] | [symbol] | [symbol] | [symbol] | [symbol] |

As shown in Table 10, in the <50 years old treatment group (carbachol 3% plus brimonidine 0.2%), the mean near visual acuity (NVA) in both eyes improved significantly from J-7.29±1.2 before treatment to J-3±1.36 at 1 hour (P<0.0001), J-4.29±1 at 2 hours (P<0.0001), J-4.57±1.2 at 4 hours (P<0.0001), J-4.86±1.17 at 8 hours (P <0.0001) and J-5±1.36 at 10 hours (P<0.0001) post-treatment.

TABLE 10
Group 3 (Hyperopic presbyopes) <50 years
3% Carbachol & brimonidine Eye drops vs Placebo vs Brimonidine

| | | | | Pre- | | | | | |
|---|---|---|---|---|---|---|---|---|---|
| | | | | Treatment | Post-Treatment | | | | |
| | | | | Near | NVa | NVa | Nva | Nva | Nva |
| | Patients′ | Age | | VA | 1 h | 2 h | 4 h | 8 h | 10 h |
| | number | (yrs) | sex | (J) | (J) | (J) | (J) | (J) | (J) |
| Carbachol 3% plus | 1 | 44 | m | 10 | 3 | 4 | 4 | 6 | 6 |
| brimonidine 0.2% | 2 | 43 | m | 8 | 3 | 5 | 6 | 6 | 6 |
| | 3 | 41 | f | 6 | 2 | 4 | 3 | 3 | 3 |
| | 4 | 45 | f | 8 | 2 | 5 | 6 | 6 | 5 |
| | 5 | 44 | m | 7 | 6 | 6 | 6 | 6 | 7 |
| | 6 | 41 | m | 6 | 3 | 3 | 3 | 4 | 4 |
| | 7 | 44 | m | 6 | 2 | 3 | 4 | 4 | 4 |
| | 8 | 44 | f | 8 | 3 | 4 | 4 | 5 | 6 |
| | 9 | 45 | m | 8 | 3 | 4 | 5 | 5 | 6 |
| | 10 | 44 | m | 6 | 2 | 4 | 3 | 3 | 3 |
| | 11 | 46 | m | 8 | 2 | 5 | 6 | 6 | 5 |
| | 12 | 44 | f | 6 | 6 | 6 | 6 | 6 | 7 |
| | 13 | 45 | m | 8 | 3 | 3 | 4 | 4 | 4 |
| | 14 | 44 | m | 7 | 2 | 4 | 4 | 4 | 4 |
| | [symbol] | [symbol] | | [symbol] | [symbol] | [symbol] | [symbol] | [symbol] | [symbol] |
| Placebo Drops | 15 | 48 | M | 8 | 8 | 8 | 8 | 8 | 8 |
| | 16 | 46 | F | 5 | 4 | 5 | 5 | 5 | 5 |
| | 17 | 47 | F | 6 | 6 | 6 | 6 | 6 | 6 |
| | 18 | 49 | M | 8 | 7 | 8 | 8 | 8 | 8 |
| | 19 | 46 | F | 6 | 6 | 6 | 6 | 6 | 6 |
| | 20 | 44 | M | 6 | 5 | 6 | 6 | 6 | 6 |
| | [symbol] | [symbol] | | [symbol] | [symbol] | [symbol] | [symbol] | [symbol] | [symbol] |
| Brimo | 21 | 43 | F | 6 | 5 | 6 | 6 | 6 | 6 |
| | 22 | 45 | F | 6 | 5 | 6 | 6 | 6 | 6 |
| | 23 | 47 | F | 6 | 5 | 5 | 6 | 6 | 6 |
| | 24 | 44 | M | 5 | 5 | 5 | 5 | 5 | 5 |
| | 25 | 49 | F | 8 | 7 | 8 | 8 | 8 | 8 |
| | 26 | 45 | M | 5 | 5 | 5 | 5 | 5 | 5 |
| | 27 | 46 | F | 6 | 6 | 6 | 6 | 6 | 6 |
| | [symbol] | [symbol] | | [symbol] | [symbol] | [symbol] | [symbol] | [symbol] | [symbol] |

No statistically significant difference in mean NVA was found between ≥50 and <50 groups before treatment (P=0.8) and at 1 hour (P=0.2), 2 hours (P=0.4), 4 hours (P=0.2), 8 hours (P=0.3) and 10 hours (P=0.27) post-treatment.

The best corrected distance visual acuity in both eyes was 20/20 in all subjects before treatment and remained at 20/20 at all time periods after treatment.

No statistically significant difference in mean NVA was found in the placebo or 0.2% brimonidine alone groups before treatment and at any time point after treatment.

All emmetropic and myopic presbyopic subjects who received carbachol plus brimonidine abandoned the use of eyeglasses. None would use placebo or brimonidine drops alone. All subjects reported that the drops did not improve their near vision, so they discontinued using the drops.

24 out of 30 hyperopic presbyope subjects (80%) who were given carbachol plus brimonidine drops abandoned the use of eyeglasses both for far and near vision. Four subjects (13.4%) only used eyeglasses for near vision with 2 to 3 dioptres less than those required before treatment according to their original hypermetropia. Only two subjects (6.6%) abandoned treatment. They indicated that the glasses would give them better near vision. None would use placebo or brimonidine drops alone, since all subjects felt no difference and discontinued using the drops.

No serious adverse ocular effects were observed during the study period for the participants being treated with carbachol plus brimonidine drops. A mild burning sensation was noted in 5.5% of all groups. Dull headaches and brow ache during the first couple of days were reported in 10% of all subjects. Temporary difficulty in low luminosity for the first couple of weeks was reported in all groups but more frequently in hyperopic subjects (40%). However, those subjects reported that these symptoms were mild and temporary and did not induce them to discontinue the drops. 97.8% of the treated subjects of all groups indicated that they would use the drops to treat their presbyopia. They showed satisfaction with both near and distance vision. The drops showed excellent safety and stability. There was no evidence of tolerance or tachyphylaxis and the effect of the drops persisted during the entire follow-period period.

A mild burning sensation was reported in 10 subjects receiving brimonidine drops. No adverse symptoms were reported in the placebo group.

In the masked study, 100% of subjects of group 1 and 2 liked and would use carbachol plus brimonidine drops if available. In group 3, 80% of subjects abandoned the use of eyeglasses, 13.4% only used eyeglasses for near vision with 2 to 3 dioptres less than those required before treatment according to their original hypermetropia, and 6.6% abandoned treatment. None would use placebo or brimonidine alone. There was no evidence of tolerance or tachyphylaxis during the follow up period.

Carbachol plus brimonidine seems to be an acceptable and safe alternative to corrective lenses and surgical procedures.

As discussed above, this study used carbachol of various concentrations and an alpha agonist (0.2% brimonidine) to improve vision in participants with refractive errors. Placebo or brimonidine drops alone were used as control. The technique is based on creating a pinhole effect pharmacologically, increasing the depth of focus from a smaller pupil and the resultant vision in the eye is clear. In monocular treatment, the vision in the fellow eye with the normal pupil might have some blurry near vision, but distant objects are clear and there is no diminished light perception. When the images are merged, most subjects of group 1 and 2 had clear focus at near distance and far distance with no perception of dimness. In group 3, temporary dimness was reported in 40% of subjects during the first couple of weeks. This was attributed to the bilateral treatment and higher concentration of carbachol (3%) used in these eyes. However, those subjects reported that these symptoms were mild and temporary and did not induce them to discontinue the drops.

Carbachol and brimonidine can be used once daily to achieve a 10-hour effect. Brimonidine has little effect on the photopic pupil, but has been effectively used for many years to prevent excessive pupil dilatation in the dark, and thereby reduces scotopic symptoms, usually from the peripheral cornea after refractive surgery. The study found a synergistic effect between carbachol and brimonidine in treating presbyopia, as well as myopia and hyperopia. Distance vision is preserved so that there are no monovision symptoms; the treatment of only one eye in some participants minimizes symptoms of dimness; synergism permits use of lower doses of miotics and reduces symptoms of headache, and brimonidine eliminates any tendency of the parasympathomimetic to cause hyperemia.

In this study, there was no evidence of tolerance or tachyphylaxis and the effect of the drops (carbachol plus brimonidine) persisted during the three months of treatment. No ocular complications were detected in any treated eyes during the entire follow-up period.

The pharmacological treatment of refractive errors including presbyopia, myopia and hyperopia using carbachol and brimonidine is an acceptable and safe alternative to spectacles and contact lenses—monofocal or multifocal, or any other surgical options. The combination of carbachol and brimonidine can improve reading vision for many presbyopic subjects. This study showed that carbachol and brimonidine improved both regular distance vision and reading and patients no longer needed glasses that were previously needed full time. Therefore, this combination treatment also improves low non-presbyopic hyperopes and myopes. This treatment can also be used to treat other refractive problems. The possibility of this pharmacological treatment opens a new therapeutic approach for subjects with refractive errors, allowing them good accommodation over time.

Pilocarpine and brimonidine similarly can treat refractive errors, including presbyopia, myopia, and hyperopia.

### Example 7

This study compared the efficacy of a formulation containing both carbachol and brimonidine with separate formulations of carbachol and brimonidine being administered at the same time. The same participants received the combination formulation and the separate formulations, with a one week washout period between the administrations.

The study tested and compared in a blind study the effectiveness of using a parasympathomimetic drug (3% carbachol) and an alpha agonist (0.2% brimonidine) in both combined and separate forms to create optically beneficial miosis to pharmacologically improve vision in presbyopia.

A prospective, blind, randomized clinical trial utilized ten naturally emmetropic and presbyopic subjects between 42 years and 58 years old with an uncorrected distance visual acuity of at least 20/20 in both eyes without additional ocular pathology. Participants were volunteers selected at random. Presbyopia is considered present if an uncorrected end-point print size ≥Jaeger (J) 5 improved by ≥1 optotype with the use of a lens ≥+1.00 D. All subjects were in good physical and ocular health and completed a questionnaire to ascertain any contraindications for participation or predisposition to complications (e.g. heart or respiratory conditions, migraines, high myopia, ocular or systemic medications, or ocular surgeries). All subjects had a fully dilated eye examination before they were considered eligible for the study. The examination screened for contraindications to the drugs, susceptibility to retinal detachment, ocular pathology, or peripheral retinal degeneration.

The inclusion criteria included age between 41 and 60 years, presbyopia (uncorrected end-point print size ≥Jaeger (J) 5 improved by ≥1 optotype with the use of a lens ≥+1.00 D), emmetropia (cycloplegic spherical equivalent (SE), ±0.25 D; astigmatism, ≤0.25 D) and binocular uncorrected distance visual acuity ≥20/20. Exclusion criteria included patients with myopia, hyperopia and astigmatism which is higher than 0.25 diopter, and corneal, lens, vitreous opacities, pupil irregularities, anisocoria, amblyopia, chronic general pathologies and medications that would interact unfavorably with carbachol and brimonidine,

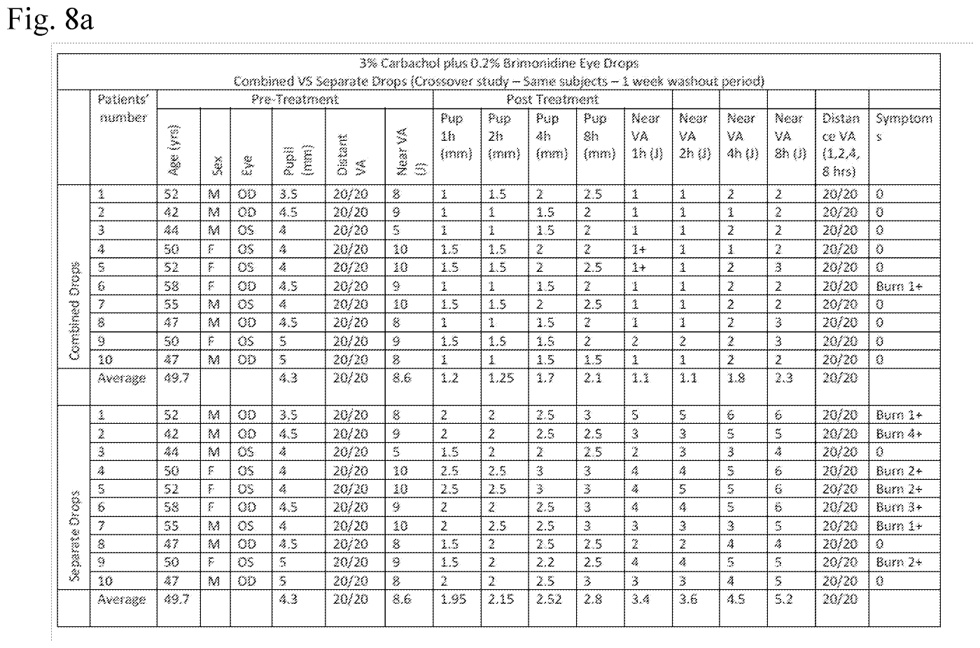

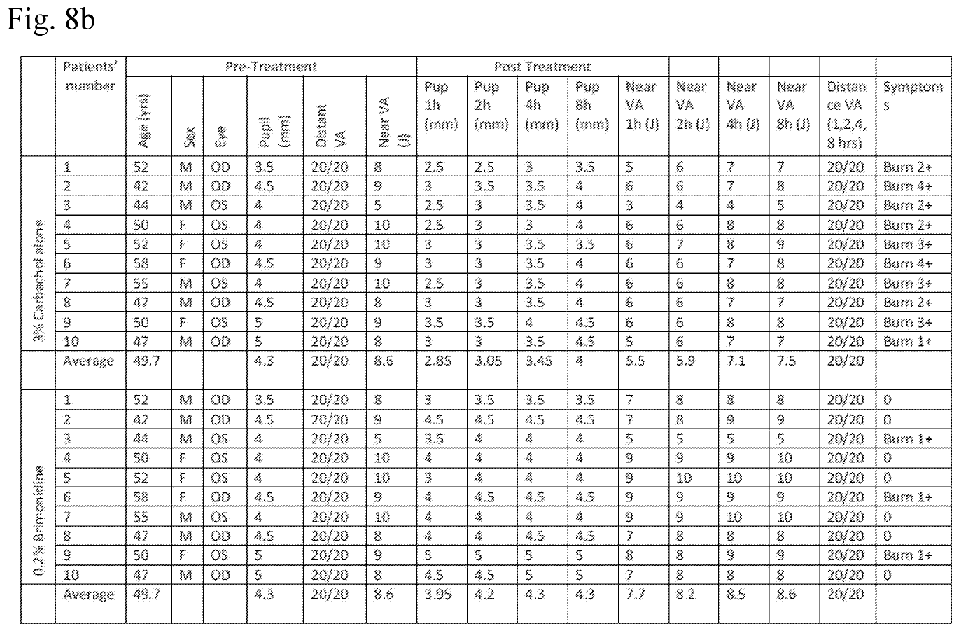

All subjects received a single dose 3% carbachol and 0.2% brimonidine in both combined and separate forms in their non-dominant eye in a crossover manner with one week washout between tests. In the separate form, carbachol was administered first followed by brimonidine after 5 minutes. The subjects' pupil size and both near and distance visual acuities were evaluated pre- and post-treatment at 1, 2, 4, and 8 hours, by a masked examiner at the same room illumination. Additionally, all subjects received just a single dose of 3% carbachol or just a dose of 0.2% brimonidine. FIG. 8a-8b shows the data from the study. All subjects were monitored to evaluate dosage, satisfaction, adverse effects and complications.

The study used standard Snellen projector chart to measure distance visual acuity. Near visual acuity was assessed at 40 cm using Jaeger (J) Eye Chart. Statistical analysis was performed using the Student's t-test and p value of less than 0.05 was considered statistically significant. Data was expressed as mean, range, and standard deviation (SD).

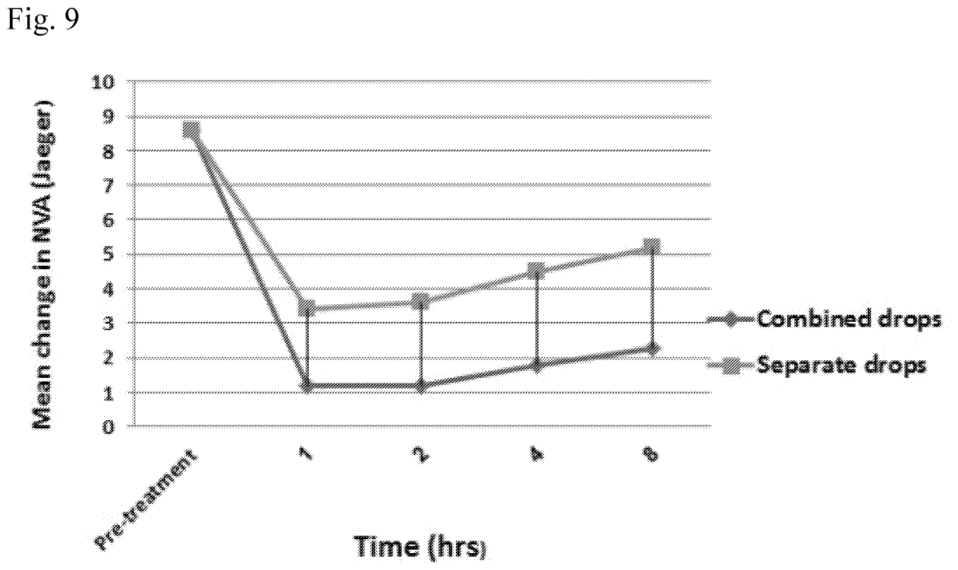

FIG. 9 shows the distribution of mean change in near visual acuity (J) over time for the same presbyopic subjects receiving 3% carbachol plus 2% brimonidine in both combined and separate forms. The mean change in NVA between pre-treatment and immediately after treatment was much larger when the participants were administered the combination drops. The change continued to be larger with the administration of the combination drops for the entire eight hour data collection period.

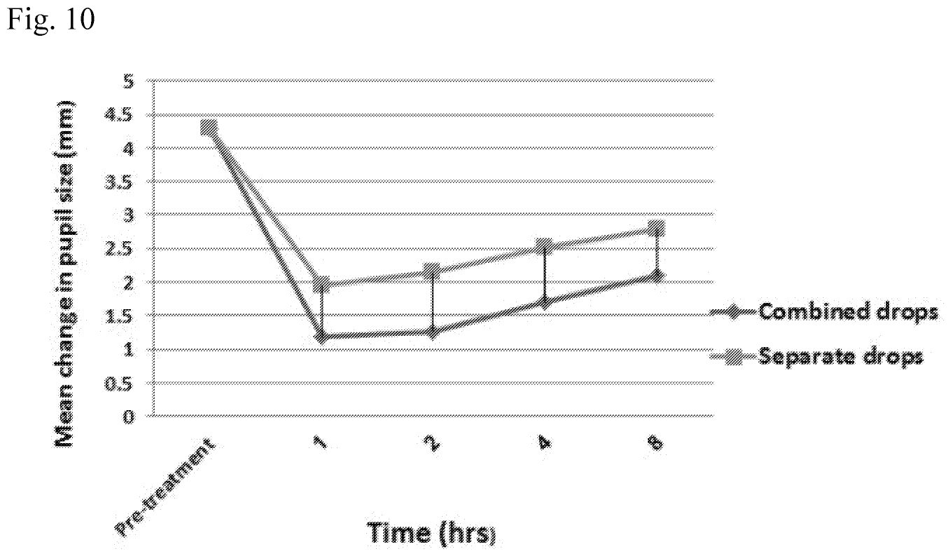

FIG. 10 shows the distribution of mean change in pupil size (mm) over time for the same presbyopic subjects receiving 3% carbachol plus 2% brimonidine in both combined and separate forms. The mean change in pupil size between pre-treatment and immediately after treatment was larger when the participants were administered the combination drops. The mean change continued to be larger with the administration of the combination drops for the entire eight hour data collection period.

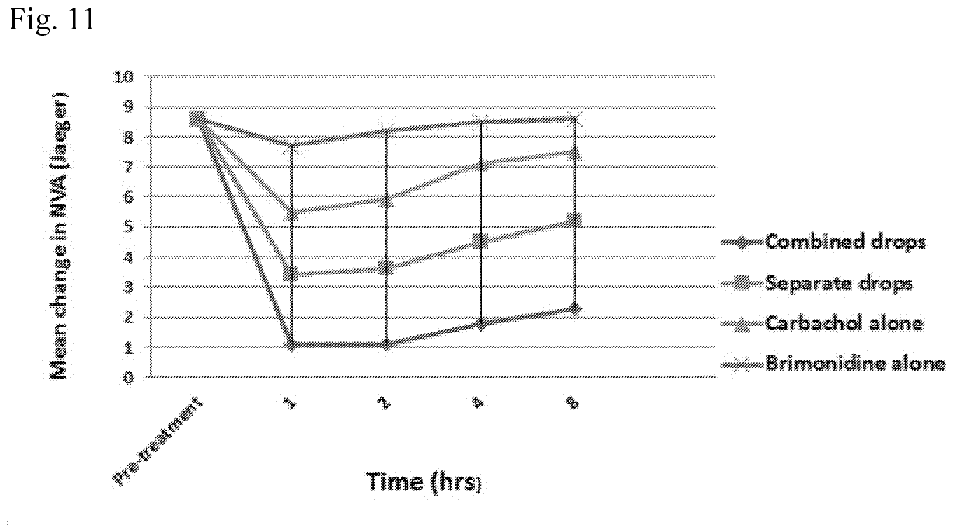

FIG. 11 shows a comparison of the distribution of mean change in near visual acuity (J) over time between the combination drops, separately administered drops, brimonidine alone and carbachol alone. The mean change was the least in participants treated with brimonidine alone, and the most in patients treated with the combination drops.

The combination drops had a synergistic effect, improving near visual acuity better than the carbachol and brimonidine administered separately.

### Example 8

Dose range studies were performed for carbachol and pilocarpine. Pilocarpine concentrations of 0.5% and 1% were compared to pilocarpine with brimonidine 0.2% and placebo. Carbachol concentrations of 1.5%, 2.25%, and 3% were compared to carbachol with brimonidine 0.2% and placebo. Pilocarpine and carbachol were also compared to each other, with and without brimonidine.

Twelve subjects in Group 1 were given either 0.5% pilocarpine plus brimonidine 0.2%, 1.0% pilocarpine plus brimonidine 0.2% or placebo eye drops in a masked fashion in their non-dominant eye.

Twelve subjects in Group 2 were given one of three strengths of carbachol 1.5%, 2.25%, or 3%, alone, or in combination with 0.2% brimonidine or placebo eye drops in a masked fashion in their non-dominant eye.

Both Groups 1 and 2 received the same pre- and post-treatment work-up and measurements: Age, gender, initial pupil size, and near vision acuity (NVA) were documented. Subjects' NVA was measured post treatment at 1, 2, 4, and 8 hours, at the same room illumination Any adverse symptoms and subject satisfaction with near and distance vision were documented.

For 0.5% pilocarpine plus 0.2% brimonidine or carbachol plus 0.2% brimonidine, 78 patients were evaluated across 3 centers. Thirty two patients were evaluated for pilocarpine plus brimonidine and 46 patients were evaluated for carbachol and brimonidine.

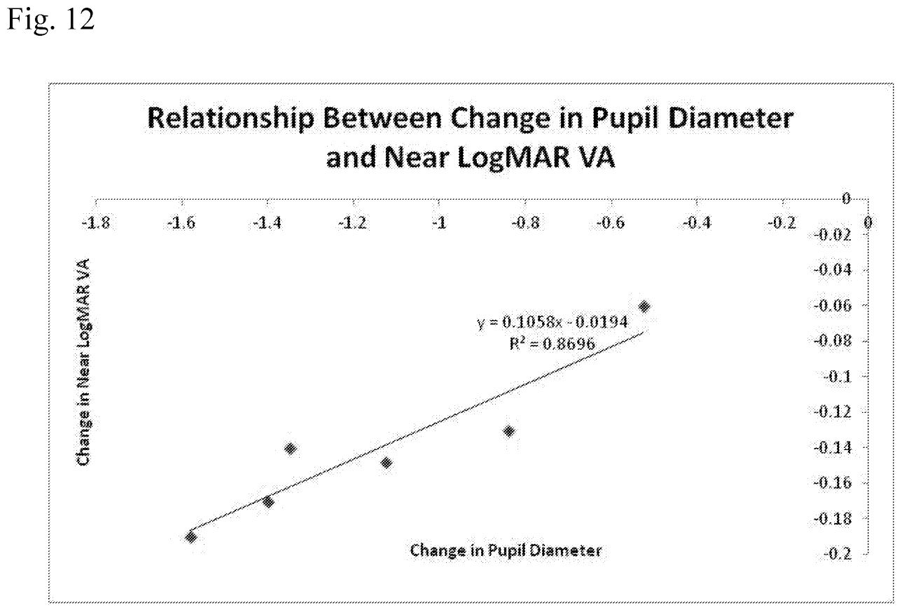

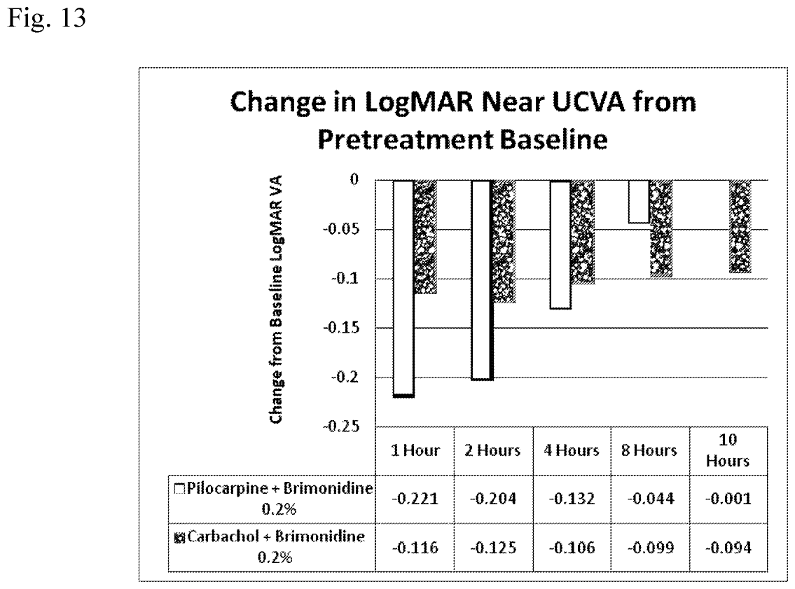

As shown in FIGS. 12 and 13, across the pilocarpine plus brimonidine and pilocarpine groups there was a clear relationship between a change from baseline for pupil diameter and a change in VA. The LogMAR activity improved in patients treated with pilocarpine or carbachol, plus brimonidine. The change in LogMAR VA favored pilocarpine for hours 1 and 2. The change in LogMAR VA favored carbachol for hours 8 and 10.

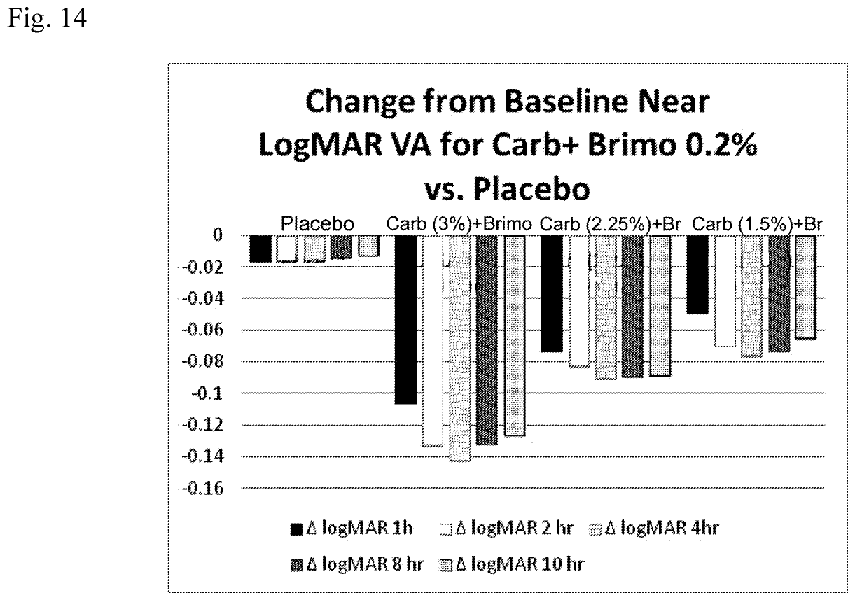

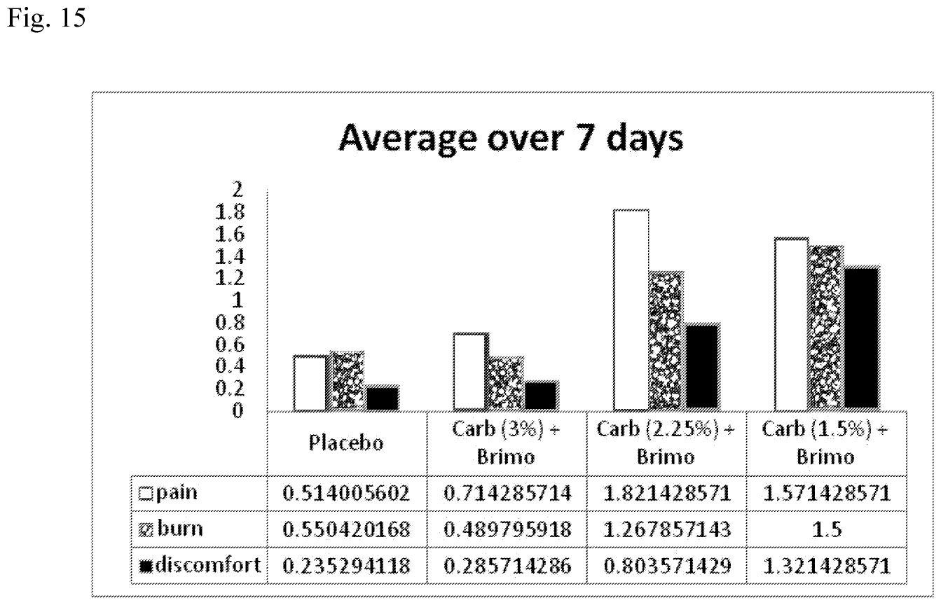

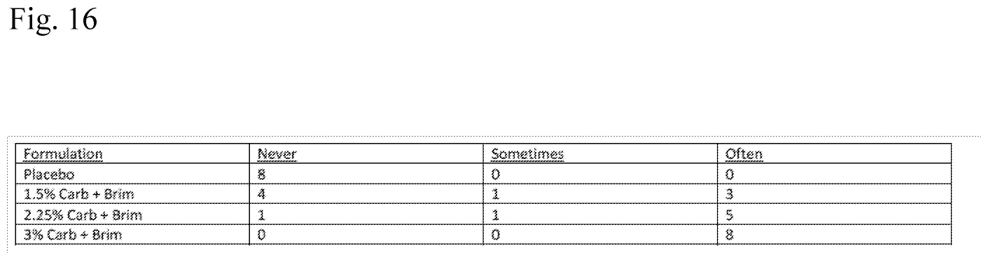

For combinations of carbachol plus brimonidine, 40 patients were evaluated (Placebo: N=17, Carb 1.5%+brim: N=8, Carb 2.25%+brim: N=8, Carb 3%+brim: N=7). A dose response was observed for carbachol when added to brimonidine, as shown in FIG. 14. An inverse dose response for carbachol was observed for tolerability, as shown in FIG. 15. The results of a post-treatment satisfaction survey for the carbachol patients is shown in FIG. 16. The survey asked the patients whether they would use the drops again.

The results of these studies showed that brimonidine is synergistic with both carbachol and pilocarpine. It also showed that both pilocarpine and carbachol are effective treatments. The combination of brimonidine and carbachol was more active and lasted longer than brimonidine and pilocarpine. Pupil size was directly correlated with improved near vision. The combination led to significant improved reading vision. The treatment did not cause symptoms of dimness, since the other eye filled in the brightness. The treatment did not interfere with distance or intermediate vision or cause monovision symptoms. The treatment can be used with glasses if the improved visual acuity is not enough for a special task. While pilocarpine has a more immediate effect, carbachol has an 8 hour duration. Increased concentrations of carbachol caused some increased discomfort. Pilocarpine was unstable at neutral pH (needs about pH 5), burned, and had a shorter duration.

No significant adverse events occurred. Mild drop-associated discomfort was noted in 10-30% of all groups (including placebo). Ninety percent of subjects indicated that they would use the active drops to treat their presbyopia, if available.

While a 0.2% brimonidine concentration was used in this study, 0.15% or 0.1%, or even less, should provide adequate synergies with pilocarpine or carbachol. Lower concentrations of brimonidine have proven to have a “whitening” effect on the eye. All of the carbachol concentrations in the study (1.5%, 2.25% and 3.0% produced improved vision.

### Example 9

Another study used pilocarpine combined with brimonidine to make one pupil smaller for several hours without surgery, relieving presbyopia and improving optical errors without glasses.

Male and female participants of any race, ages 45 to 60, with a +/− sphere 0.5 D with +/−0.5 D correction for reading, were chosen for inclusion. Individuals with allergies or adverse reactions to pilocarpine or brimonidine, individuals with glaucoma, cataracts, ocular infection, ocular inflammation, retinal tears, retinal disease, ocular surgery within the past 30 days, individuals that wear contact lenses, individuals that used any eye drops within the past 7 days, individuals that were pregnant or nursing, and individuals that had participated in any other clinical trial within the past 30 days, were excluded from the study.

Twenty volunteers were studied on 3 separate days 1 week apart to allow for washout. Routine external examination and refraction for distance and reading (if current refraction has been determined no longer than 60 days ago, those values were used) was also performed. Intraocular pressures was also measured (Goldman applanation), as well as a dilated examination of the lens and retina.

There were 3 study days. Each day, each of the 3 groups of 5 patients received different study medications. After a washout of 7 days, the study was repeated with the groups using another test medication, so that after 3 study days each patient was tested with each medication and each patient was their own control. Each patient was studied in the same room with the same illumination by the same examiner each time they were studied.

After determination of the volunteer's non-dominant eye, volunteers were randomized to 3 groups and treated with drops in the nondominant eye only. The doses tested were: 1% pilocarpine, 0.2% brimonidine, and 1% pilocarpine plus 0.2% brimonidine. For the combination eye drops, pilocarpine was administered first and brimonidine 5 minutes later. Only one drop of each was administered once. Hourly, for 8 hours, under mesopic illumination, patients were asked to read the eye chart and pupil diameters measured at each time with an infrared pupilometer.

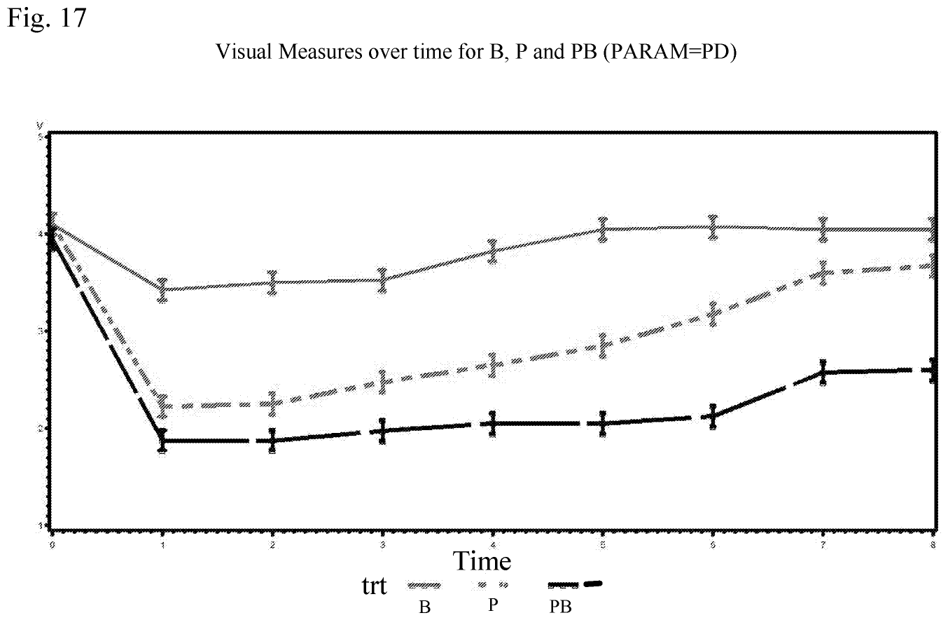

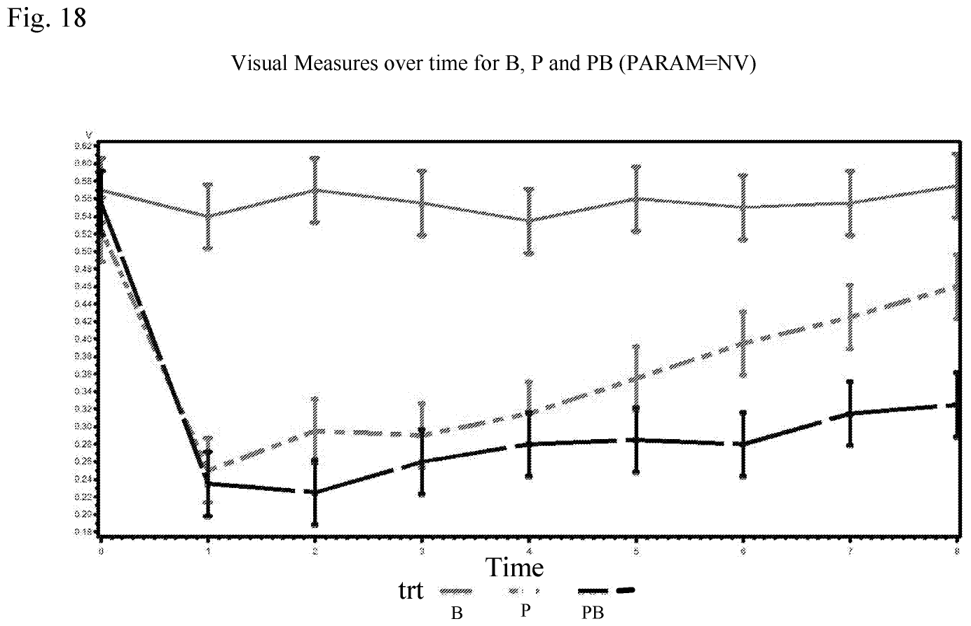

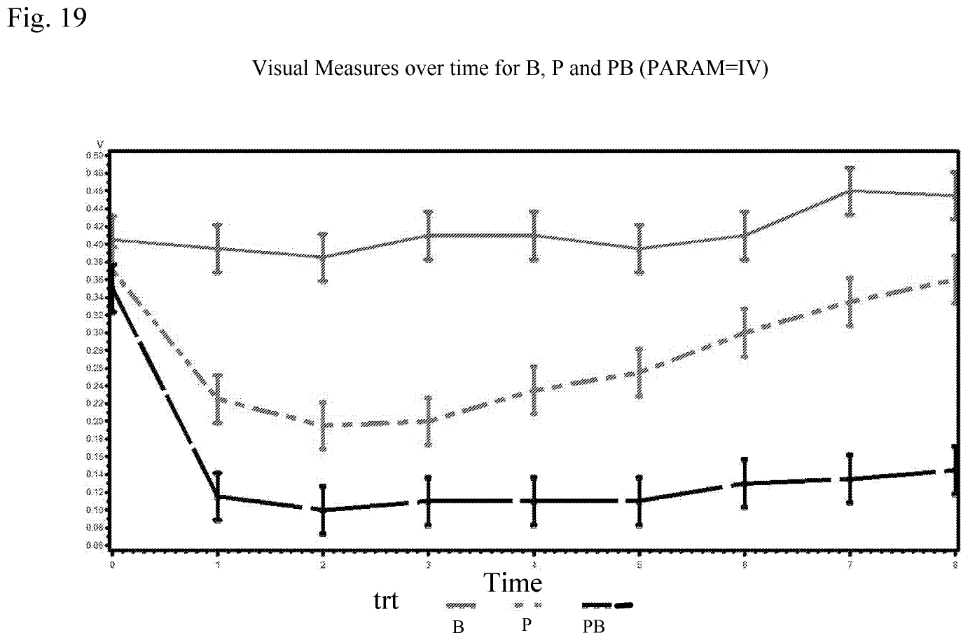

The results are shown in FIGS. 17-19. The figures show visual measures of pupil dilation, near vision, and intermediate vision, over time respectively for brimonidine alone, pilocarpine alone, and 1% pilocarpine plus 0.2% brimonidine. Both near vision and intermediate vision were significantly improved with the pilocarpine plus brimonidine drops compared to either brimonidine or pilocarpine alone.

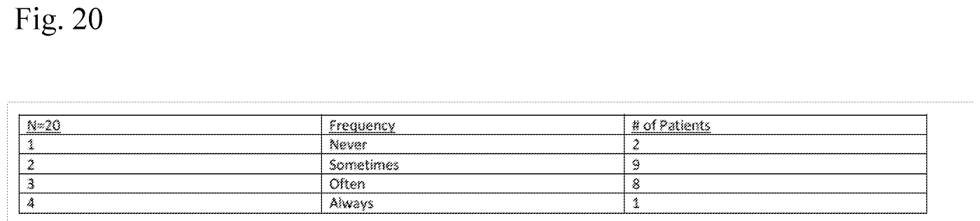

The twenty patients were surveyed after receiving the pilocarpine plus brimonidine treatment. More specifically, they were asked whether, if these drops were available, would they use them instead of glasses? 90% of subjects indicated that they would use the drops to treat presbyopia if available, as shown in FIG. 20.

### Example 10

Another study tested patients that had intraocular lenses (IOL) implanted in the eye (pseudophakia) and needed reading glasses post surgery. Patients typically have this type of surgery to treat cataracts and correct distance vision.

In this study, fifteen patients, ages 38 to 80, underwent pseudophakia surgery to correct their distance vision. One of the patients had surgery in both eyes, while the remaining fourteen had the surgery in a single eye.

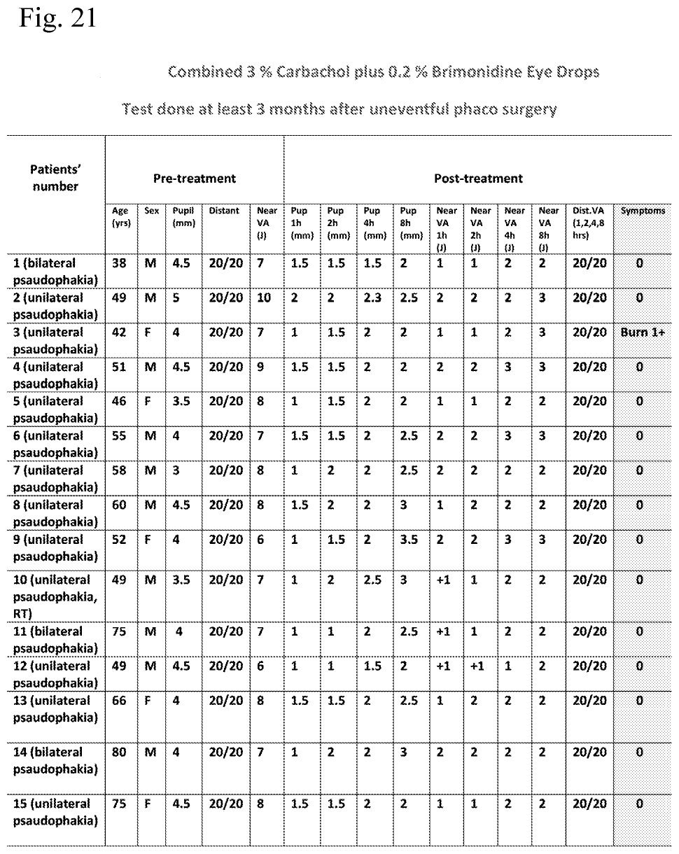

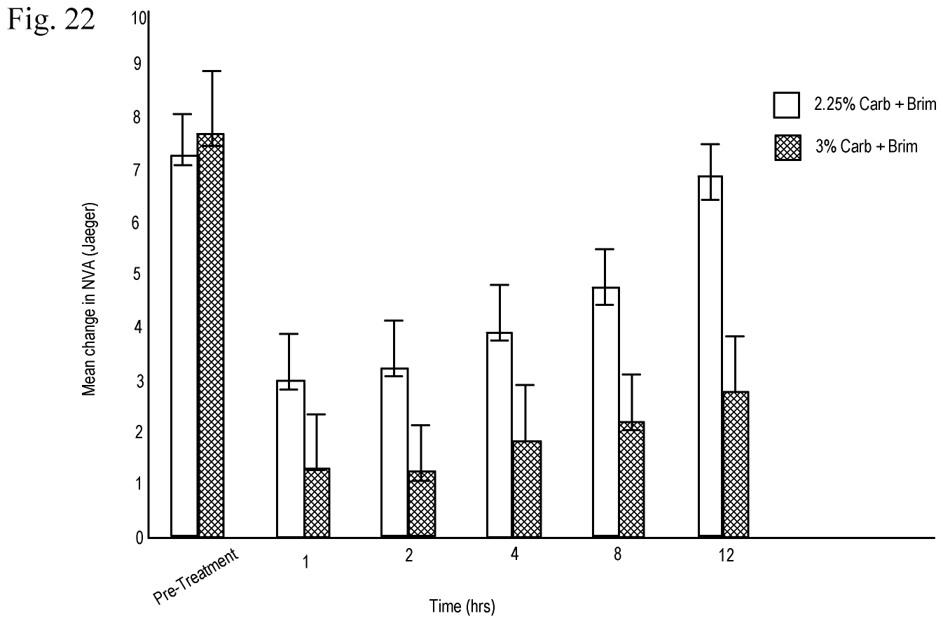

At least three months after uneventful surgery, the presbyopic patients were given a single combined eye drop containing 3% carbachol plus 0.2% brimonidine in one eye. The results are shown in FIG. 21.

For all of the patients, their distance vision before and after the drops were administered was 20/20. In all of the patients, their pupil size decreased dramatically post treatment with the drops. In addition, their NVA was greatly enhanced post treatment with the drops, throughout the entire eight hour period following administration of the drops. Only one patient reported burning as a side effect.

This study showed that refractive errors resulting from pseudophakia were corrected by a combined formulation of carbachol and brimonidine for at least eight hours. In spite of surgical and pharmacological manipulation during surgery, these drops still worked to correct near vision in these patients. An ophthalmic preparation in the form of an eyedrop lets cataract patients, many of which are elderly, see without reading glasses.

All of the patent and non-patent references discussed herein are incorporated herein by reference.

TABLE 11

| | | | Pre-Treatment | | | | | | | | | Pilo Alone | | | | | | |
|---|---|---|---|---|---|---|---|---|---|---|---|---|---|---|---|---|---|---|
| | | | Right Eye | | | Left Eye | | | Pupil Diam | | Near | Pilo | 1 hr | | 2 hrs | | 4 hrs. | |
| # | Age | Sex | Sph | Cyl | Axis | Sph | Cyl | Axis | O.D. | O.S. | Va(j) | [%] | pupil | nVA(j) | pupil | nVA(j) | pupil | nVA(j) |
| 1 | 47 | M | 0 | 0 | 0 | 0 | 0 | 0 | 4.5 | 4.5 | 3 | 0.25 | 3.5 | 2 | 4 | 3 | 4 | 3 |
| 2 | 54 | M | 0 | 0 | 0 | 0 | 0 | 0 | 5.5 | 5.4 | 5 | 0.25 | 2.5 | 3 | 3.5 | 3 | 4 | 5 |
| 3 | 49 | M | 0 | 0 | 0 | 0 | 0 | 0 | 4 | 4 | 6 | 0.25 | 3.3 | 5 | 3 | 5 | 4 | 6 |
| 4 | 54 | F | 0 | −0.5 | 20 | 0.5 | −0.5 | 160 | 3.5 | 3.5 | 8 | 0.25 | 3 | 6 | 3.5 | 6 | 4 | 7 |
| 5 | 53 | F | 0 | 0 | 0 | 0 | 0 | 0 | 4 | 4 | 6 | 0.5 | 2.5 | 3 | 3 | 5 | 3.3 | 5 |
| 6 | 47 | F | 0 | 0 | 0 | 0 | 0 | 0 | 4 | 4.3 | 5 | 0.5 | 3.8 | 3 | 4 | 3 | 4 | 3 |
| 7 | 53 | F | 0 | 0 | 0 | 0 | −0.5 | 25 | 3.5 | 3.5 | 6 | 0.5 | 3 | 6 | 2.5 | 5 | 3 | 5 |
| 8 | 52 | F | 0 | 0 | 0 | 0 | 0 | 0 | 5 | 5 | 6 | 0.5 | 3 | 5 | 3.3 | 3 | 4.2 | 5 |
| 9 | 52 | F | 0 | 0 | 0 | −0.5 | −0.5 | 135 | 4.3 | 3.9 | 5 | 1 | 2.5 | 3 | 3 | 3 | 4 | 5 |
| 10 | 53 | F | 0 | 0 | 0 | 0 | 0 | 0 | 3 | 3 | 8 | 1 | 1.5 | 3 | 1.5 | 2 | 2.5 | 5 |
| 11 | 53 | F | 0 | 0 | 0 | −0.5 | −0.25 | 175 | 4.5 | 4.5 | 5 | 1 | 2.5 | 2 | 2.5 | 2 | 3 | 3 |
| 12 | 52 | F | 0 | 0 | 0 | 0 | 0 | 0 | 4.5 | 4.5 | 10 | 1 | 3 | 8 | 3 | 8 | 4 | 10 |
| | | | | | | | | | | | | Pilo + Brimonidine | | | | | | |
| | | | | | | | | | | | | Pilo | 1 hr. | | 2 hrs | | 4 hrs | |
| | | | | | | | | | | | # | [%] | pupil | nVA(j) | pupil | nVA(j) | pupil | nVA(j) |
| | | | | | | | | | | | 1 | 0.25 | 3 | 2 | 2.5 | 2 | 3.5 | 3 |
| | | | | | | | | | | | 2 | 0.25 | 3.3 | 3 | 3.5 | 3 | 4 | 5 |
| | | | | | | | | | | | 3 | 0.25 | 2.5 | 3 | 3 | 5 | 3.5 | 8 |
| | | | | | | | | | | | 4 | 0.25 | 2 | 5 | 3 | 6 | 3.5 | 6 |
| | | | | | | | | | | | 5 | 0.5 | 2 | 3 | 2.5 | 3 | 3.5 | 5 |
| | | | | | | | | | | | 6 | 0.5 | 3 | 3 | 3 | 3 | 3.75 | 3 |
| | | | | | | | | | | | 7 | 0.5 | 2.5 | 5 | 3 | 5 | 3 | 5 |
| | | | | | | | | | | | 8 | 0.5 | 3 | 5 | 3 | 5 | 3 | 5 |
| | | | | | | | | | | | 9 | 1 | 3 | 2 | 3 | 3 | 3.5 | 4 |
| | | | | | | | | | | | 10 | 1 | 2 | 3 | 2 | 2 | 2.5 | 3 |
| | | | | | | | | | | | 11 | 1 | 2 | 2 | 2 | 3 | 2.5 | 2 |
| | | | | | | | | | | | 12 | 1 | 3 | 5 | 3 | 4 | 4 | 3 |

### Example 11

One study examined the use of carbachol with an alpha agonist (brimonidine) on the outcome of presbyopia treatment (Influence of Different Concentrations of Carbachol Drops on the Outcome of Presbyopia Treatment—A Randomized Study, Abdelkader, International Journal of Ophthalmic Research 2019 September; 5(1):317-320, herein incorporated by reference). This study aimed at investigating the optimal dose of carbachol to effectively improve near vision in presbyopic subjects for a prolonged duration of time.

A prospective, double-masked, randomized study included fifty seven emmetropic and presbyopic subjects between 44 and 60 years of age with an uncorrected distance visual acuity of at least 20/20 in both eyes without additional ocular pathology. Presbyopia was considered present if an uncorrected end-point print size ≥Jaeger (J) 5 improved by ≥1 optotype with the use of a lens ≥+1.00 D. Subjects were divided into 2 groups. Group 1 (n=32 eyes) received a single dose of 2.25% carbachol plus 0.2% brimonidine eye drops. Group 2 (n=25 eyes) received a single dose of 3% carbachol plus 0.2% brimonidine eye drops. Drops were given to all subjects in their non-dominant eye. The subjects' pupil size and both near and distance visual acuities were evaluated pre- and post-treatment at 1, 2, 4, 8 and 12 hours, by a masked examiner at the same room illumination.

Statistically significant improvement in near visual acuity (NVA) was seen in all subjects who received both concentrations of carbachol plus brimonidine drops (P<0.0001). Significant and sustained improvement in mean NVA was reported in higher concentrations of carbachol drops than in lower concentrations (P<0.0001). No serious adverse ocular effects were observed in any of the subjects of either group. The higher concentration of carbachol was found to be safe and provided greater efficacy in improving near visual acuity than lower concentrations with prolonged duration of action.

Patients with myopia, hyperopia and astigmatism higher than 0.25 diopter as well as those with corneal, lens and vitreous opacities, pupil irregularities, anisocoria, amblyopia, chronic general pathologies and medications that would interact unfavorably with carbachol and brimonidine were excluded.

The mean age of group 1 (2.25% carbachol) was 51.1±4.5 years (range, 44-55 years); 18 males and 14 females. The mean age of group 2 (3% carbachol) was 52.8±3.9 years (range, 47-60 years); 14 males and 11 females. In the treatment group, the number of subjects ≥50 years was 16 and those <50 years was 14. No statistically significant difference in mean age or sex was found among the 2 groups.

In group 1, the mean near visual acuity (NVA) improved significantly from J 7.37±1.6 before treatment to J 2.96±0.8 at 1 h, J 3.34±1.1 at 2 h, J 3.93±0.98 at 4 h, and J 4.98±0.85 at 8 h post-treatment (p<0.0001). At 12 h post-treatment, mean NVA was 6.75±1.58 J (p=0.11). The mean pupil size (PS) decreased significantly from 4.74±0.47 mm before treatment to 2.68±0.41 mm at 1 h, 3±0.37 mm at 2 h, 3.35±0.4 mm at 4 h and 3.58±0.43 mm at 8 h post-treatment (p<0.0001). At 12 h post-treatment, mean pupil size was 4.51±69 mm (p=0.12).

In group 2, the mean near visual acuity (NVA) improved significantly from J 7.72±1.48 before treatment to J 1.36±0.56 at 1 h, J 1.4±0.57 at 2 h, J 1.8±0.58 at 4 h, J 2.32±0.47 at 8 h and 2.64±0.7 at 12 h post-treatment (p<0.0001). The mean pupil size (PS) decreased significantly from 4.55±0.55 mm before treatment to 1.2±0.25 mm at 1 h, 1.34±0.31 mm at 2 h, 1.64±0.3 mm at 4 h, 2±0.28 mm at 8 h and 2.27±0.34 mm at 12 h post-treatment (p<0.0001).

In group 2 when 3% carbachol was instilled, the improvement in near visual acuity was statistically significant up to 12 h post-treatment whereas in group 1, the improvement in near visual acuity was only significant up to 8 h post-treatment. Significant improvement in mean NVA was reported in 3% carbachol and brimonidine drops than 2.25% concentration (p<0.0001).

The mean change in near visual acuity (NVA)(Jaeger) and pupil size (PS) (mm) over time for group 1 (2.25% carbachol plus brimonidine) versus group 2 (3% carbachol plus brimonidine) is shown in Table 12 below.

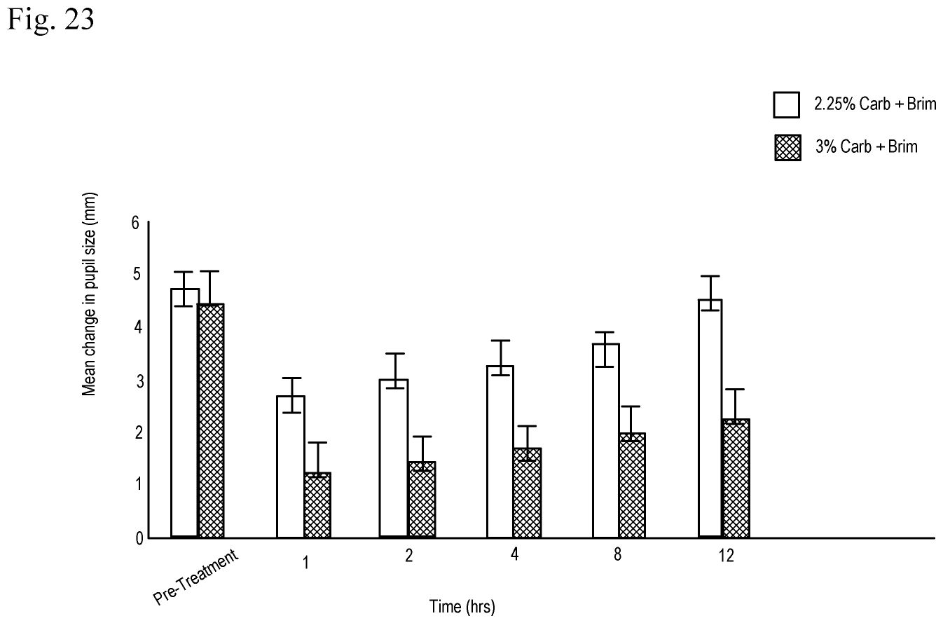

FIGS. 23-23 show the mean change in near visual acuity (Jaeger) and pupil size (mm) over time for groups 1 and 2.

The composition of drops used in Group 1 and Group 2 also contained 100 ppm of benzalkonium chloride (BAK or BAC).

Burning sensation, brow ache, dimness or any other serious adverse ocular effects were not observed in any of the patients of both groups. Systemic side effects such as bradychardia, bronchospasm, and digestive problems were not found.

The uncorrected distance visual acuity was 20/20 of both eyes in all subjects before treatment and remained at 20/20 at all periods after treatment.

TABLE 12

| Time | | Group 1 | Group 2 | P-value |
|---|---|---|---|---|
| Pre- | NVA | 7.37 | 7.72 | 0.4 |
| treatment | PS | 4.74 | 4.55 | 0.1 |
| 1-h | NVA | 2.96 | 1.36 | P < 0.0001 |
| | PS | 2.68 | 1.2 | P < 0.0001 |
| 2-h | NVA | 3.34 | 1.4 | P < 0.0001 |
| | PS | 3 | 1.34 | P < 0.0001 |
| 4-h | NVA | 3.93 | 1.8 | P < 0.0001 |
| | PS | 3.35 | 1.64 | P < 0.0001 |
| 8-h | NVA | 4.68 | 2.32 | P < 0.0001 |
| | PS | 3.58 | 2.04 | P < 0.0001 |
| 12-h | NVA | 6.75 | 2.64 | P < 0.0001 |
| | PS | 4.51 | 2.27 | P < 0.0001 |

Statistically significant improvement in mean near visual acuity (NVA) and mean pupil size (PS) was achieved in all subjects who received both concentrations of carbachol plus brimonidine drops (p<0.0001). Significant improvement in mean NVA and PS up to 12 hours post treatment was reported in all subjects received 3% carbachol drops (p<0.0001). No serious adverse ocular effects were observed in higher concentrations of carbachol.

While the concentration of carbachol between the two groups was different, a difference of 0.75%, the improvement of mean NVA and PS up to 12 hours of post treatment was seen.

Based on the data, higher concentration of carbachol was found to be safe and provided greater efficacy in improving near visual acuity than lower concentration with prolonged duration of action.

### Example 12

In a clinical study, thirty hyperopic subjects between the ages of 41 and 52 years of age were divided into two groups. The mean average of the groups were 47.5±3.7 years (range, 41-52 years). No statistically significant difference in mean age. Group 1 received bilateral dosing of 3% carbachol eye drops. Group 2 received bilateral dosing of 3% carbachol plus 0.2% brimonidine eye drops. Drops were given to all subjects in both eyes. The subjects' pupil size and both near and distance visual acuities were evaluated pre- and post-treatment at 1, 2, 4, 8 and 12 hours, by a masked examiner at the same room illumination.

24 out of 30 subjects (80%) abandoned the use of eyeglasses both for far and near vision and four subjects (13.4%) only used eyeglasses for near vision with 2 to 3 diopters less than those required before treatment. Only two hyperopic subjects (6.6%) abandoned treatment.

### Example 13

One study examined the user of combined versus separate carbachol and brimonidine drops in corrected presbyopia (Clinical outcomes of combined versus separate carbachol and brimonidine drops in corrected presbyopia, Abdelkader et al., Eye and Vision 2016; 3:31, herein incorporated by reference). This study aimed at improving near vision in presbyopic subjects by testing and comparing in a masked fashion the efficacy of using a parasympathomimetic drug (3% carbachol) and an alpha-2 agonist (0.2% brimonidine) in both combined and separate forms to create optically beneficial miosis to pharmacologically improve vision in presbyopia.

A prospective, double-masked, randomized, controlled clinical trial was conducted. Ten naturally emmetropic and presbyopic subjects between 42 and 58 years old with uncorrected distance visual acuity of at least 20/20 in both eyes without additional ocular pathology were eligible for inclusion. All subjects received 3% carbachol and 0.2% brimonidine in both combined and separate forms, 3% carbachol alone and 0.2% brimonidine (control) alone in their non-dominant eye in a crossover manner with one week washout between tests. The subjects' pupil sizes and both near and distance visual acuities will be evaluated pre- and post-treatment at 1, 2, 4, and 8 h, by a masked examiner at the same room illumination.

Statistically significant improvement in mean near visual acuity (NVA) was achieved in all subjects who received combined 3% carbachol and 0.2% brimonidine in the same formula compared with those who received separate forms or carbachol alone or brimonidine alone (P<0.0001). The combined solution demonstrated greater efficacy than the other solutions that were tested. Improving the depth of focus by making the pupil small caused statistically significant improvement in near visual acuity, with no change in binocular distance vision.

Participants were randomly selected volunteers. Presbyopia was considered present if an uncorrected end-point print size ≥Jaeger (J) 5 improved by ≥1 optotype with the use of a lens ≥+1.00 D. All subjects were screened to be in good physical and ocular health and they completed a questionnaire to ascertain any contra-indications for participation or predisposition to complications (e.g., heart or respiratory conditions, migraines, high myopia, ocular or systemic medications, or ocular surgeries). All subjects had a fully dilated eye examination before they were considered eligible for the study. The examination screened for contraindications to the drugs, susceptibility to retinal detachment, ocular pathology, or peripheral retinal degeneration. Inclusion criteria were as follows: age between 42 and 58 years, emmetropia [cycloplegic spherical equivalent (SE), ±0.25 D; astigmatism, ≤0.25 D] and binocular uncorrected distance visual acuity ≥20/20. Exclusion criteria included patients with myopia, hyperopia and astigmatism higher than 0.25 D as well as those with corneal, lens and vitreous opacities, pupil irregularities, anisocoria, amblyopia, chronic general pathologies and medications that would interact unfavorably with carbachol and brimonidine. None of the patients included in the study had received any topical medication that could cause pupil mydriasis or miosis. During the study, the subjects were closely monitored and regularly asked to report on any ocular, systemic, or physiological reactions they experienced. Atropine was available in the event of adverse effects, although none was reported. All procedures followed were in accordance with the ethical standards of the responsible committee on human experimentation.

A single dose of 3% carbachol together with 0.2% brimonidine in both combined and separate forms and 3% carbachol alone or 0.2% brimonidine alone (control) were instilled in the non-dominant eye of the same ten emmetropic presbyopic subjects with one week washout between tests. In a separate form, carbachol was instilled first followed by brimonidine after 5 min. In the single dose of combined 3% carbachol together with 0.2% brimonidine, 100 ppm of benzalkonium chloride was present. The 3% carbachol drops included 50 ppm of benzalkonium chloride. The 0.2% brimonidine drops included 50 ppm of benzalkonium chloride.

Initial pupil size and both near and distance visual acuities were documented before treatment and at 1, 2, 4, and 8 h after treatment by the same independent examiner in the same room with the same instruments. Distance visual acuity was measured using the standard Snellen projector chart at 4 m. Near visual acuity (NVA) was assessed at 40 cm using a hand-held Rosenbaum chart with Jaeger notation, always employing the same luminosity of 160 cd/m2. Pupil size (PS) was measured using Colvard handheld Infrared pupilometer.

Ten naturally emmetropic and presbyopic subjects with a mean age of 49.7±4.8 years (range, 42-58 years) were eligible for inclusion. These subjects (6 males and 4 females) with an uncorrected distance visual acuity of at least 20/20 in both eyes were without additional ocular pathology.

In the combined drops group, the mean near visual acuity (NVA) improved significantly from J 8.6±1.5 before treatment to J 1.1±0.3 at 1 h, J 1.1±0.3 at 2 h, J 1.8±0.4 at 4 h and J 2.3±0.5 at 8 h post-treatment (P<0.0001). The mean pupil size (PS) decreased significantly from 4.3±0.5 mm before treatment to 1.2±0.3 mm at 1 h, 1.2±0.3 mm at 2 h, 1.7±0.2 mm at 4 h and 2.1±0.3 mm at 8 h post-treatment (P<0.0001).

In the separate drops group, the mean NVA improved significantly from J 8.6±1.5 before treatment to J 3.4±1 at 1 h (P=0.0002), J 3.6±1 at 2 h (P=0.0002), J4.5±1 at 4 h (P=0.0004) and J 5.2±0.8 at 8 h (P=0.0008) post-treatment. The mean (PS) decreased significantly from 4.3±0.5 mm before treatment to 1.9±0.3 mm at 1 h, 2.2±0.2 mm at 2 h, 2.5±0.3 mm at 4 h and 2.8±0.2 mm at 8 h post-treatment (P<0.0001).

In the 3% carbachol alone group, the mean NVA improved significantly from J8.6±1.5 before treatment to J5.5±1 at 1 h (P=0.001), J 5.9±0.8 at 2 h (P=0.001), J7±1.2 at 4 h (P=0.007) and J 7.5±1 at 8 h (P=0.027). The mean (PS) decreased significantly from 4.3±0.5 mm before treatment to 2.8±0.3 mm at 1 h (P=0.0002), 3±0.3 mm at 2 h (P=0.0002), 3.5±0.3 mm at 4 h (P=0.0007). At 8 h post-treatment, mean (PS) was 4±0.3 mm (P=0.15).

In the 0.2% brimonidine alone group, no statistically significant difference in mean NVA and mean (PS) was found before treatment and at any time point after treat-ment (P>0.05).

Significant improvement in mean NVA was reported in combined 3% carbachol and brimonidine drops than separate forms or carbachol alone or brimonidine alone (P<0.0001).

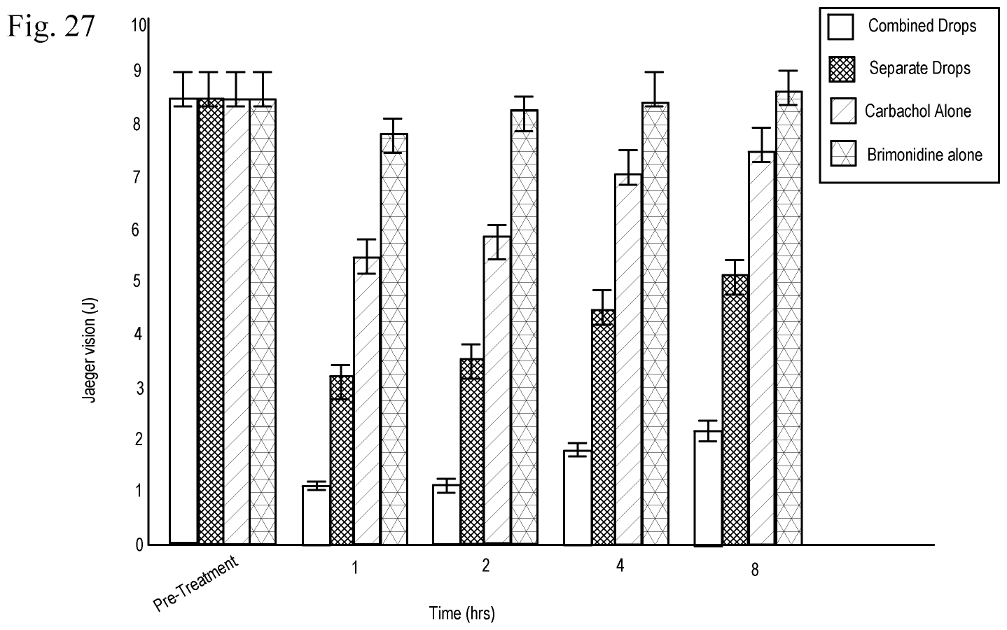

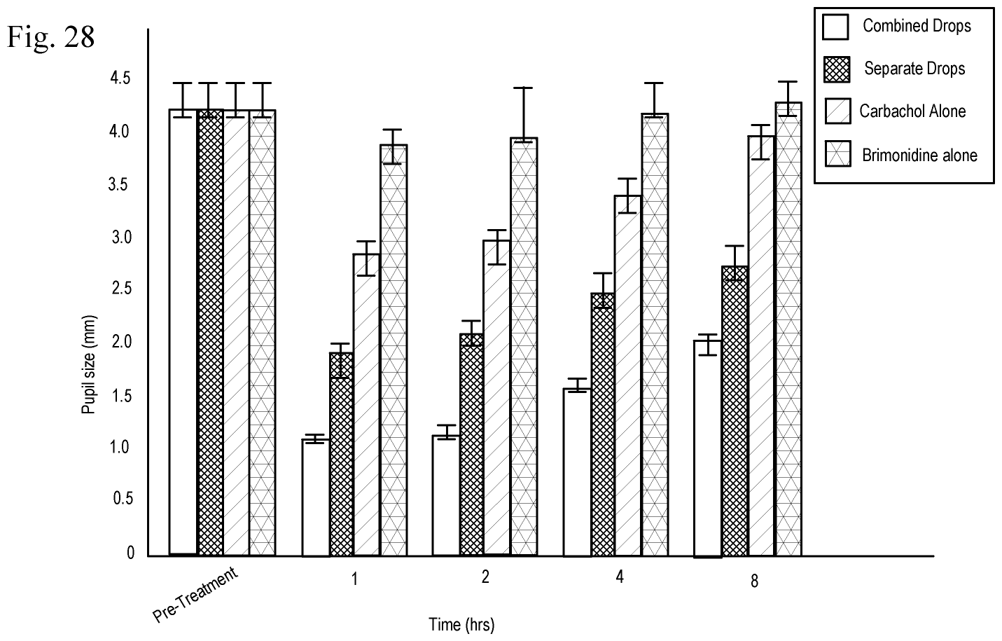

FIGS. 27-28 show the distribution mean change in near visual acuity over time and mean change in pupil size of the study.

The data from the study is shown in Table 13 below.

TABLE 13

| | | | | | | P-value | | |
|---|---|---|---|---|---|---|---|---|
| | | | | 3% | 0.2% | Combined | Combined | Combined |
| | | Combined | Separate | Carbachol | Brimonidine | vs | vs 3% | vs 2% |
| Time | | Drops | Drops | alone | alone | separate | Carbachol | Brimonidine |
| Pre- | NVA | 8.6 ± 1.5 | 8.6 ± 1.5 | 8.6 ± 1.5 | 8.6 ± 1.5 | 1 | 1 | 1 |
| treatment | PS | 4.3 ± 0.5 | 4.3 ± 0.5 | 4.3 ± 0.5 | 4.3 ± 0.5 | 1 | 1 | 1 |
| 1-h | NVA | 1.1 ± 0.3 | 3.4 ± 1 | 5.5 ± 1 | 7.7 ± 1.3 | P <0.0001 | P <0.0001 | P <0.0001 |
| | PS | 1.2 ± 0.3 | 1.9 ± 0.3 | 2.8 ± 0.3 | 3.95 ± 0.5 | P <0.0006 | P <0.0001 | P <0.0001 |
| 2-h | NVA | 1.1 ± 0.3 | 3.6 ± 1 | 5.9 ± 0.8 | 8.2 ± 1.3 | P <0.0001 | P <0.0001 | P <0.0001 |
| | PS | 1.2 ± 0.3 | 2.2 ± 0.2 | 3 ± 0.3 | 4.2 ± 0.4 | P <0.0001 | P <0.0001 | P <0.0001 |
| 4-h | NVA | 1.8 ± 0.4 | 4.5 ± 1 | 7 ± 1.2 | 8.5 ± 1.4 | P <0.0001 | P <0.0001 | P <0.0001 |
| | PS | 1.7 ± 0.2 | 2.5 ± 0.3 | 3.5 ± 0.3 | 4.3 ± 0.5 | P <0.0001 | P <0.0001 | P <0.0001 |
| 8-h | NVA | 2.3 ± 0.5 | 5.2 ± 0.8 | 7.5 ± 1 | 8.6 ± 1.5 | P <0.0001 | P <0.0001 | P <0.0001 |
| | PS | 2.1 ± 0.3 | 2.8 ± 0.2 | 4 ± 0.3 | 4.3 ± 0.5 | P <0.0006 | P <0.0001 | P <0.0001 |

No subject complained of the Pulfrich effect, which occurs due to intraocular differences in retinal illuminance inducted by anisocoria. All subjects in our pilot study reported that they could drive safely day and night without distortions in the perception of any movement.

Significant improvement in near visual acuity was found to be higher in all subjects who received combined 3% carbachol and brimonidine in the same formula compared with those who received separate forms or carbachol alone or brimonidine alone (P<0.0001).

The study attributed the marked improvement in near visual acuity in subjects receiving the combined formula to the penetration enhancers (benzalkonium chloride and carboxymethylcellulose) that were added to the combined formula and perhaps also to the fact that when the receptors of iris dilator and constrictor muscles are both acted upon at the same time, they reinforce each other than when one is stimulated before the other permitting maximal effect with less to overcome.

The study showed that brimonidine tartrate 0.2% alone produced a mild miotic effect mainly during the first hour after instillation under light luminance conditions but this did not reach statistical significance (P>0.05). In monocular treatment, the vision in the fellow eye with the normal pupil will have some blurry near vision, but distant objects are clear and there is no diminished light perception.

The study concluded that monocular pharmacologic treatment of presbyopia with one drop a day of carbachol and brimonidine in the non-dominant eye permits acceptable reading vision for many presbyopes even in older subjects.

In Examples 11-13, various concentrations of carbachol alone, brimonidine alone and carbachol plus brimonidine have been tested. A preservative of benzalkonium chloride was added. In carbachol and brimonidine alone, 50 ppm of benzalkonium chloride was added. In carbachol plus brimonidine, 100 ppm of benzalkonium chloride was added.

Prior art has taught that benzalkonium chloride has known toxic effects and should be used cautiously. The prior art additionally teaches away from using benzalkonium chloride concentrations over 100 ppm due to potential damage to corneal epithelium cells.

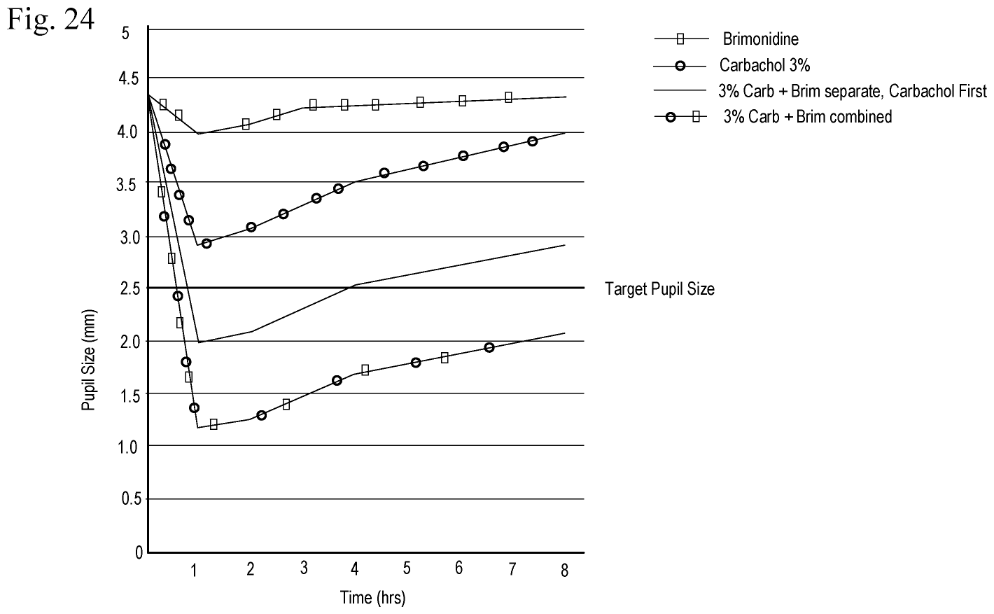

However, during testing of combined 3% carbachol plus 0.2% brimonidine with 100 ppm of benzalkonium chloride drops, separate administration of 3% carbachol with 50 ppm of benzalkonium chloride and then administration of 0.2% brimonidine, administration of just 3% carbachol with 50 ppm of benzalkonium chloride, and administration of just 0.2% brimonidine resulted in mean pupil size in emmetropic presbyopes of reaching a target pupil size of ≤2.5 mm in hours 1-8 only in subjects that were administered the 3% carbachol plus 0.2% brimonidine with 100 ppm of benzalkonium chloride drops. Subjects administered drops of just 3% carbachol with 50 ppm of benzalkonium chloride never achieved the target pupil size during hours 1-8 as shown in FIG. 24.

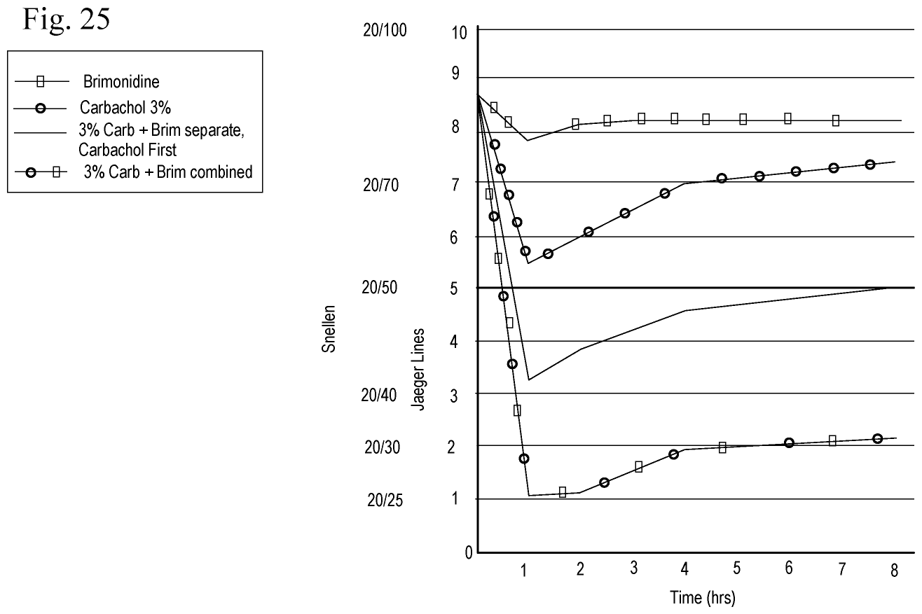

During testing of combined 3% carbachol plus 0.2% brimonidine with 100 ppm of benzalkonium chloride drops, separate administration of 3% carbachol with 50 ppm of benzalkonium chloride and then administration of 0.2% brimonidine, administration of just 3% carbachol with 50 ppm of benzalkonium chloride, and administration of just 0.2% brimonidine resulted in mean near visual acuity (NVA) in emmetropic presbyopes of ≥20/40 in hours 1-8 only in subjects that were administered the 3% carbachol plus 0.2% brimonidine with 100 ppm of benzalkonium chloride drops. Subjects administered drops of just 3% carbachol with 50 ppm of benzalkonium chloride never achieved an NVA of ≥20/40 as shown in FIG. 25.

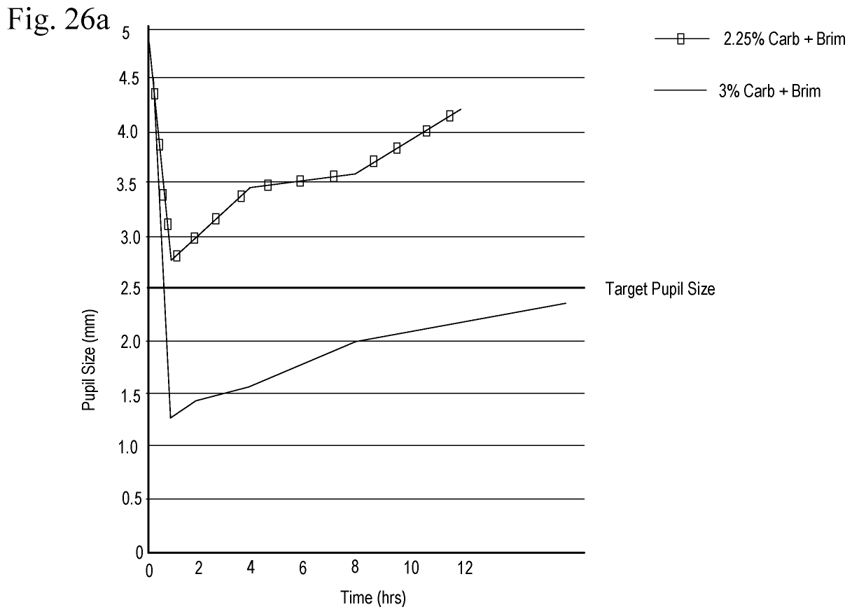

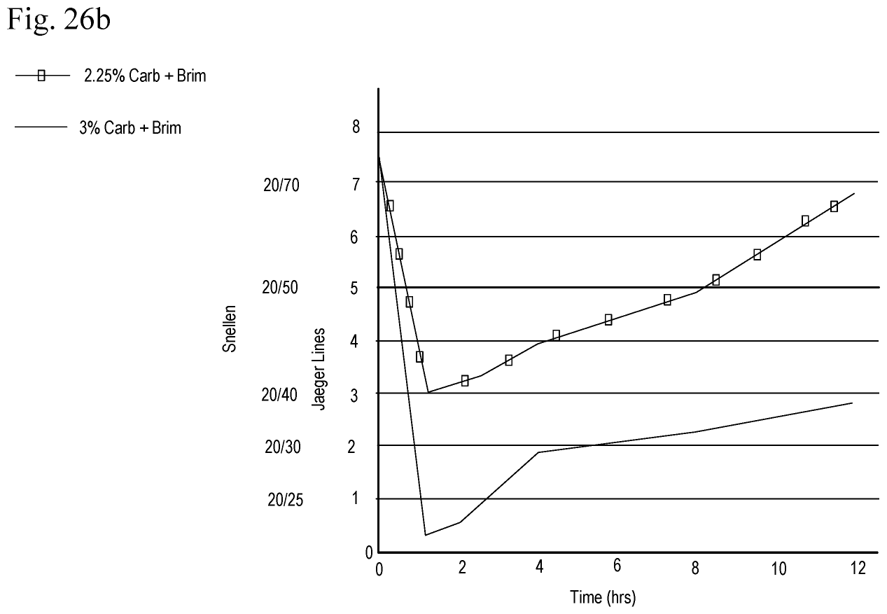

FIGS. 26a-26b show the highly significant twelve hour effect on pupil size and NVA. The combination of 3% carbachol with 0.2% brimonidine with 100 ppm of benzalkonium chloride achieved the target pupil size of ≤2.5 mm in 1-12 hours over the combination drops of 2.25% carbachol with 0.2% brimonidine with 100 ppm. The combination of 3% carbachol with 0.2% brimonidine with 100 ppm of benzalkonium chloride achieved an NVA of ≥20/40 in 1-12 hours over the combination drops of 2.25% carbachol with 0.2% brimonidine with 100 ppm.

The much greater pharmacodynamic effect in magnitude and duration with the combination of carbachol, brimonidine and 100 ppm of BAK was not expected given that brimonidine alone with 50 ppm had virtually no effect on pupil size of NVA. Furthermore, the pharmacodynamic effect is particularly unexpected when noting that drops of carbachol 3% and brimonidine 0.2% when administered as a combination achieved a target pupil size of ≤2.5 mm for 8 hrs while these same actives administered separately, 5 minutes apart, with the same cumulative BAK exposure, ˜100 ppm, only reached this target for approximately 4 hrs and achieved the NVA target of 20/40 only at hour 1. Therefore, the Applicant is suggesting that the pharmacodynamic effect is not due to the additive effect of any of individual components. but the novel combination.

### Example 14

One study examined the effect of the combination treatment of carbachol and brimonidine tartrate on intraocular pressure in presbyopic adults. Since brimonidine reduces aqueous product and uveoscleral outflow, while carbachol increase outflow through the trabecular meshwork, the combination of brimonidine and carbachol would be expected to reduce IOP by at least 4 mm Hg. However, this did not occur, which was unexpected. Previous studies dosed only one eye but checked binocular vision for distance and near. Previous studies relied on the premise that if near vision is improved in one eye due to the pinhole effect, the near vision would be the same or better with both eyes as in the treated eye. However, when parasympathimimetics are dosed as the sole active ingredient, they induce ciliary body contraction and shape change in the lens similar to when reading at near. This lens change causes a significant myopic shift towards near-sightedness in many patients and blurring of distance vision.

Therefore, the strategy for use of parasympathimimetics to create a pinhole effect was to treat only one eye so that one eye would maintain good distance vision. If one eye of a subject is dosed with a combination of brimonidine and carbachol and the distance vision is binocularly tested, even if the subject lost distance vision because of a myopic shift in the dosed eye, the binocular vision at distance would still be 20/20 because one eye remained untreated. The study below monocularly tested the same eye that received the dose of brimonidine and carbachol for intraocular pressure and distance vision. The study provides evidence that the distance vision is not impacted by a formulation of 3% carbachol and 0.2% brimonidine tartrate, which was unexpected as carbachol alone results in distance loss. It is novel that distance vision is preserved if both eyes are treated. It should also be noted that even though miotics alone can transiently increase and then decrease IOP in healthy subjects, there was no evidence of an IOP increase or decrease in the combination of brimonidine and carbachol. The study is discussed below.

A prospective single-arm clinical trial was conducted. Sixteen subjects between 42 to 58 years old (mean=49.5) years, including 9 males and 7 females were enrolled. The sixteen subjects were presbyopic, as defined by uncorrected end-point print size ≥Jaeger (J) 5 improved by ≥1 optotype with the use of a lens ≥+1.00 D; emmetropic, as defined by cycloplegic spherical equivalent±0.25 D and astigmatism ≤0.25 D; had uncorrected distance visual acuity of at least 20/20 in both eyes; were without additional ocular pathology; and were in general good health. All subjects received with 3% carbachol and 0.2% brimonidine tartrate. Study drug was applied topically to the non-dominant eye; the dominant eye was untreated and served as a control.

Intraocular pressure (IOP) was measured using a handheld tonometer (Tono-Pen). The mean of 4 measurements was taken and those with bad signals or extreme readings were discarded. At baseline, all subjects were normotensive and has a mean IOP of 13.8 mm HG in the treated eye and 14.5 mm HG in the control eye. No significant changes in IOP were observed in either eye. The results of this single-dose study indicate no significant effect on IOP when 3% carbachol and 0.2% brimonidine tartrate are administered in combination to normotensive subjects with presbyopia. This is a particularly important finding for a treatment that could be used widely in patients with undiagnosed ocular hypertension of glaucoma in whom fluctuations in IOP would be undesirable. The results of the study are shown in Table 14.

TABLE 14

| | | | | | | Post Treatment | | | | | | | | | | |
|---|---|---|---|---|---|---|---|---|---|---|---|---|---|---|---|---|
| | Pretreatment | | | | | | | | | | | | | | | Distance |
| | | | IOP | | | IOP (mmHG) Treatment eyes | | | | | IOP (mmHG) Control | | | | | VA (1, 2, |
| Patient | Age | | treated | IOP | Distance | IOP | IOP | IOP | IOP | IOP | IOP | IOP | IOP | IOP | IOP | 4, 8, |
| Numbers | (yrs) | Sex | eyes | control | VA | 1 h | 2 h | 4 h | 8 h | 24 h | 1 h | 2 h | 4 h | 8 h | 24 h | 24 hrs) |
| 1 | 55 | M | 13 | 15 | 20/20 | 13 | 13 | 12 | 13 | 12 | 15 | 15 | 15 | 16 | 16 | 20/20 |
| 2 | 42 | M | 17 | 17 | 20/20 | 16 | 16 | 17 | 16 | 15 | 17 | 17 | 16 | 16 | 17 | 20/20 |
| 3 | 44 | M | 16 | 14 | 20/20 | 15 | 15 | 17 | 17 | 15 | 14 | 14 | 14 | 14 | 14 | 20/20 |
| 4 | 50 | F | 11 | 13 | 20/20 | 10 | 11 | 11 | 11 | 11 | 12 | 12 | 13 | 12 | 12 | 20/20 |
| 5 | 52 | F | 16 | 16 | 20/20 | 16 | 16 | 16 | 15 | 16 | 15 | 16 | 16 | 16 | 15 | 20/20 |
| 6 | 58 | F | 14 | 15 | 20/20 | 13 | 14 | 12 | 13 | 13 | 16 | 15 | 16 | 15 | 16 | 20/20 |
| 7 | 55 | M | 19 | 17 | 20/20 | 18 | 18 | 18 | 18 | 17 | 17 | 17 | 17 | 16 | 16 | 20/20 |
| 8 | 47 | M | 16 | 18 | 20/20 | 15 | 15 | 16 | 16 | 16 | 17 | 18 | 17 | 18 | 18 | 20/20 |
| 9 | 50 | F | 10 | 12 | 20/20 | 11 | 10 | 11 | 11 | 10 | 11 | 11 | 13 | 12 | 12 | 20/20 |
| 10 | 47 | M | 12 | 13 | 20/20 | 12 | 12 | 13 | 13 | 11 | 12 | 12 | 12 | 12 | 12 | 20/20 |
| 11 | 47 | M | 15 | 16 | 20/20 | 15 | 15 | 15 | 16 | 15 | 16 | 16 | 16 | 15 | 15 | 20/20 |
| 12 | 46 | F | 10 | 12 | 20/20 | 11 | 11 | 11 | 12 | 10 | 12 | 12 | 13 | 12 | 11 | 20/20 |
| 13 | 50 | F | 15 | 14 | 20/20 | 15 | 15 | 14 | 14 | 14 | 15 | 14 | 15 | 15 | 14 | 20/20 |
| 14 | 48 | F | 12 | 14 | 20/20 | 12 | 12 | 12 | 11 | 11 | 13 | 13 | 13 | 15 | 15 | 20/20 |
| 15 | 55 | M | 11 | 11 | 20/20 | 10 | 10 | 11 | 11 | 11 | 11 | 12 | 12 | 12 | 11 | 20/20 |
| 15 | 46 | M | 13 | 15 | 20/20 | 12 | 13 | 13 | 12 | 12 | 15 | 14 | 15 | 15 | 15 | 20/20 |
| Average | 49.5 | | 13.75 | 14.5 | 20/20 | 13.3 | 13.5 | 13.68 | 13.68 | 13.06 | 14.25 | 14.25 | 14.56 | 14.43 | 14.3 | 20/20 |

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for ameliorating or reducing at least one refractive error of a pseudophakic patient selected from the group consisting of myopia, hyperopia, and astigmatism, comprising:
administering to at least one eye of the patient an ophthalmic preparation comprising:
a therapeutically effective amount of one or more parasympathomimetic drugs, or pharmaceutically acceptable salts thereof; and
a therapeutically effective amount of an alpha agonist or an alpha antagonist, or pharmaceutically acceptable salts thereof.

2. The method of claim 1, wherein the alpha agonist is brimonidine.

3. The method of claim 2, wherein brimonidine is present in the preparation in an amount of approximately 0.05-0.3%.

4. The method of claim 1, wherein the parasympathomimetic drug is carbachol.

5. The method of claim 4, wherein carbachol is present in the preparation in an amount of approximately 0.5-5%.

6. The method of claim 1, wherein the parasympathomimetic drug is carbachol and the alpha agonist is brimonidine.

7. The method of claim 1, wherein the parasympathomimetic drug is pilocarpine.

8. The method of claim 7, wherein pilocarpine is present in an amount of approximately 0.25% to approximately 1.5%.

9. The method of claim 1, wherein the alpha agonist is phentolamine.

10. The method of claim 9, wherein phentolamine is present in an amount of approximately less than 2%.

11. The method of claim 1, wherein the preparation is administered to one eye.

12. The method of claim 1, wherein the preparation is administered to both eyes.

13. The method of claim 1, wherein parasympathomimetic drug and the alpha agonist are combined in a single formulation.

14. The method of claim 1, wherein the ophthalmic preparation further comprises tropicamide.

15. A method of treating at least one refractive error in a patient that has had ocular surgery, comprising:
administering to at least one eye of the patient an ophthalmic preparation comprising:
a therapeutically effective amount of one or more parasympathomimetic drugs, or pharmaceutically acceptable salts thereof; and
a therapeutically effective amount of an alpha agonist or an alpha antagonist, or pharmaceutically acceptable salts thereof.

16. The method of claim 15, wherein the alpha agonist is brimonidine.

17. The method of claim 16, wherein brimonidine is present in the preparation in an amount of approximately 0.05-0.3%.

18. The method of claim 15, wherein the parasympathomimetic drug is carbachol.

19. The method of claim 18, wherein carbachol is present in the preparation in an amount of approximately 0.5-5%.

20. The method of claim 15, wherein the parasympathomimetic drug is carbachol and the alpha agonist is brimonidine.

21. The method of claim 15, wherein the parasympathomimetic drug is pilocarpine.

22. The method of claim 21, wherein pilocarpine is present in an amount of approximately 0.25% to about 1.5%.

23. The method of claim 15, wherein the alpha agonist is phentolamine.

24. The method of claim 23, wherein phentolamine is present in an amount of approximately less than 2%.

25. The method of claim 15, wherein the preparation is administered to one eye.

26. The method of claim 15, wherein the preparation is administered to both eyes.

27. The method of claim 15, wherein parasympathomimetic drug and the alpha agonist are combined in a single formulation.

28. The method of claim 15, wherein the ophthalmic preparation further comprises tropicamide.

29. The method of claim 15, wherein the refractive error is selected from the group consisting of myopia, hyperopia, astigmatism, and any combination of myopia, hyperopia, and astigmatism.

30. The method of claim 15, wherein the surgery is laser surgery.

31. The method of claim 15, wherein the surgery includes replacing at least one natural lens with an artificial intraocular lens.

32. The method of claim 31, wherein the ophthalmic preparation temporarily restores multifocality to at least one of the eyes with the artificial intraocular lens.

33. A method of creating multifocality in a pseudophakic patient, reducing symptoms of presbyopia in a patient having an eye or both eyes comprising: administering to the eye or both eyes with the presbyopia a pharmaceutically effective amount of an ophthalmic preparation comprising at least a therapeutically effective amount of one or more parasympathomimetic drugs, or pharmaceutically acceptable salts thereof; and a therapeutically effective amount of an alpha agonist or an alpha antagonist, or pharmaceutically acceptable salts thereof.

34. The method of claim 33, wherein the alpha agonist is brimonidine.

35. The method of claim 34, wherein brimonidine is present in the preparation in an amount of approximately 0.05-0.3%.

36. The method of claim 33, wherein the parasympathomimetic drug is carbachol.

37. The method of claim 35, wherein carbachol is present in the preparation in an amount of approximately 0.5-5%.

38. The method of claim 33, wherein carbachol is present in the preparation in an amount of approximately 2.25-3.5%.

39. The method of claim 33, wherein the parasympathomimetic drug is carbachol and the alpha agonist is brimonidine.

40. The method of claim 33, wherein the alpha agonist is phentolamine.

41. The method of claim 78, wherein phentolamine is present in an amount of approximately less than 2%.

42. The method of claim 33, wherein the preparation is administered to one eye.

43. The method of claim 33, wherein the preparation is administered to both eyes.

44. The method of claim 33, wherein parasympathomimetic drug and the alpha agonist are combined in a single formulation.

45. The method of claim 33, wherein the ophthalmic preparation further comprises tropicamide.

46. The method of claim 33, wherein one or both eyes of the patient contains an artificial intraocular lens.

47. The method of claim 45, wherein the ophthalmic preparation temporarily restores multifocality to at least one of the eyes with the artificial intraocular lens.

48. The method of claim 1, wherein the ophthalmic preparation further comprises benzalkonium chloride present in an amount of approximately 0.005-0.1%.

49. The method of claim 15, wherein the ophthalmic preparation further comprises benzalkonium chloride present in an amount greater than 0.005%.

50. A method of creating multifocality in a pseudophakic patient, reducing symptoms of presbyopia in a patient having an eye or both eyes comprising: administering to the eye or both eyes with the presbyopia a pharmaceutically effective amount of an ophthalmic preparation comprising at least a therapeutically effective amount of one or more parasympathomimetic drugs, or pharmaceutically acceptable salts thereof; a therapeutically effective amount of an alpha agonist or an alpha antagonist, or pharmaceutically acceptable salts thereof; and a permeation enhancer.

51. The method of claim 50, wherein the alpha agonist is brimonidine.

52. The method of claim 50, wherein the parasympathomimetic drug is carbachol.

53. The method of claim 50, wherein the benzalkonium chloride is present in the ophthalmic preparation in an amount of approximately 0.005-0.1%.

54. A method for ameliorating or reducing at least one refractive error of a pseudophakic patient selected from the group consisting of myopia, hyperopia, and astigmatism, comprising:
administering to at least one eye of the patient an ophthalmic preparation comprising:
a therapeutically effective amount of one or more parasympathomimetic drugs, or pharmaceutically acceptable salts thereof; and
a permeation enhancer.

55. The method of claim 54, wherein the parasympathomimetic drug is carbachol.

56. The method of claim 54, wherein the permeation enhancer is benzalkonium chloride and is present in the ophthalmic preparation in an amount of approximately 0.005-0.1%.

57. The method of claim 15, further comprising a permeation enhancer of benzalkonium chloride.

58. The method of claim 57, wherein the parasympathomimetic drug is carbachol.

59. The method of claim 57, wherein the benzalkonium chloride is present in the ophthalmic preparation in an amount of approximately 0.005-0.1%.

60. A method of creating multifocality in a pseudophakic patient, reducing symptoms of presbyopia in a patient having an eye or both eyes comprising: administering to the eye or both eyes with the presbyopia a pharmaceutically effective amount of an ophthalmic preparation comprising at least a therapeutically effective amount of one or more parasympathomimetic drugs, or pharmaceutically acceptable salts thereof; and a permeation enhancer.

61. The method of claim 60, wherein the parasympathomimetic drug is carbachol.

62. The method of claim 60, wherein the benzalkonium chloride is present in the ophthalmic preparation in an amount of approximately 0.005-0.1%.

63. A method for ameliorating or reducing at least one refractive error of a pseudophakic patient selected from the group consisting of myopia, hyperopia, and astigmatism, comprising:
administering to an eye or both eyes of the patient an ophthalmic preparation comprising:
a therapeutically effective amount of one or more parasympathomimetic drugs, or pharmaceutically acceptable salts thereof; and
a therapeutically effective amount of an alpha agonist or an alpha antagonist, or pharmaceutically acceptable salts thereof;
wherein at least intermediate vision of the pseudophakic patient is improved from administration of the ophthalmic preparation to the eye or both eyes of the patient.

64. The method of claim 63, wherein the ophthalmic preparation further comprises a permeation enhancer.

65. The method of claim 63, wherein the wherein the alpha agonist is brimonidine.

66. The method of claim 63, wherein the parasympathomimetic drug is carbachol.

67. The method of claim 64, wherein the permeation enhancer is benzalkonium chloride and is present in the ophthalmic preparation in an amount of approximately 0.005-0.1%.

68. The method of claim 63, wherein the parasympathomimetic drug is pilocarpine.

69. The method of claim 63, wherein the parasympathomimetic drug is carbachol and the alpha agonist is brimonidine.

70. A method for preventing a parasympathomimetic induced myopic shift in a pseudophakic patient receiving parasympathomimetic drugs or pharmaceutically acceptable salts thereof comprising:
administering to two eyes of the pseudophakic patient an ophthalmic preparation comprising:
a therapeutically effective amount of one or more of the parasympathomimetic drugs, or pharmaceutically acceptable salts thereof; and
a therapeutically effective amount of an alpha2 agonist, or pharmaceutically acceptable salts thereof
wherein the ophthalmic preparation increases a depth of focus, and preserves distance visual acuity in the administered two eyes of the pseudophakic patient, while preventing the parasympathomimetic induced myopic shift.

71. The method of claim 70, wherein the parasympathomimetic drug is carbachol.

72. The method of claim 70, wherein the parasympathomimetic drug is carbachol and the alpha 2 agonist is brimonidine.
